(19) United States
(12) Patent Application Publication
(10) Pub. No.: US 2014/0094429 A1
(43) Pub. Date: Apr. 3, 2014

(54) FGF RECEPTOR-ACTIVATING 3-O-ALKYL OLIGOSACCHARIDES, PREPARATION THEREOF AND THERAPEUTIC USE THEREOF

(71) Applicant: Sanofi, Paris (FR)

(72) Inventors: Pierre Alexandre DRIGUEZ, Le Mesnil Le Roi (FR); Philippe DUCHAUSSOY, Toulouse (FR); Pierre FONS, Toulouse (FR)

(73) Assignee: Sanofi, Paris (FR)

(21) Appl. No.: 13/951,820

(22) Filed: Jul. 26, 2013

Related U.S. Application Data
(63) Continuation of application No. PCT/IB2012/050390, filed on Jan. 27, 2012.

(30) Foreign Application Priority Data
Jan. 27, 2011 (FR) 1150645

(51) Int. Cl. C07H 13/12

(52) U.S. Cl. CPC C07H 13/12
USPC 514/54; 536/17.5

(57) ABSTRACT

The invention relates to the FGF receptor-activating oligosaccharides corresponding to formula (I):

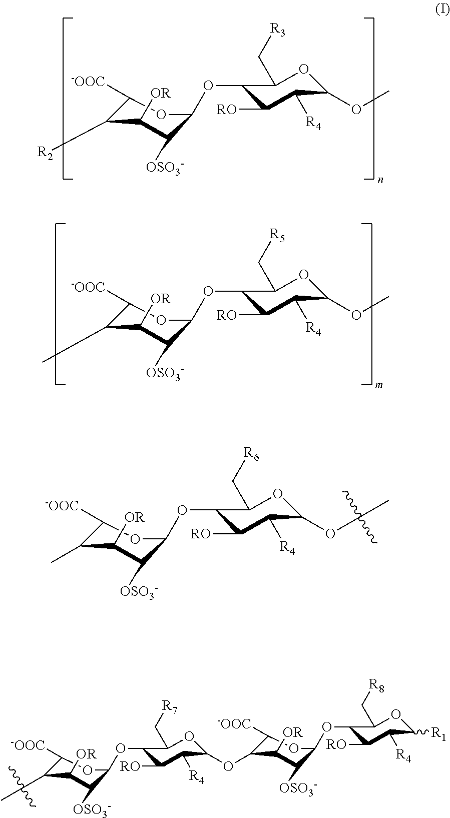

in which R₁ represents an optionally substituted —O-alkyl group, R₂ represents a hydroxyl group or an —O-alkyl group, R₃, R₅, R₆, R₇ and R₈ represent —OSO₃⁻ or hydroxyl groups, R₄ represents an —NH—CO-alkyl or —O-alkyl group, R represents an —O-alkyl group, and n and m, which may be identical to or different from one another, represent integers equal to 0 or 1.

Method for the preparation thereof and therapeutic use thereof are also provided.

### CROSS REFERENCE TO RELATED APPLICATIONS

This application is a continuation of International Patent Application No. PCT/IB2012/050390 filed Jan. 27, 2012, the entire contents of which are expressly incorporated herein by reference.

### BACKGROUND OF THE INVENTION

The present invention relates to 3-O-alkyl oligosaccharides which are agonists of the FGFs/FGFRs system, to the preparation thereof and to the therapeutic use thereof.

Angiogenesis is a process of generation of new blood capillaries. During the blockage of a blood vessel, angiogenesis, associated with arteriogenesis (dilation of the capillaries), improves the revascularization of the blocked area. It has been shown in vitro and in vivo that several growth factors, such as Vascular Endothelial Growth Factors (VEGFs) and Fibroblast Growth Factors (FGFs), stimulate the neovascularisation process.

FGFs are a family of 23 members. FGF2 (or basic FGF) is an 18 kDa protein. FGF2 induces, in endothelial cells in culture, their proliferation, their migration and the production of proteases. In vivo, FGF2 promotes neovascularisation phenomena. FGF2 interacts with endothelial cells via two classes of receptors, high-affinity receptor tyrosine kinases (FGFRs) and low-affinity receptors of heparan sulphate proteoglycan (HSPG) type.

It is known that cell surface receptor tyrosine kinases associate in dimeric form with a complex made up of two ligand molecules and one heparan sulphate molecule. The formation of this complex makes it possible to trigger a cascade of intracellular signals resulting in activation of cell proliferation and migration, which are two key processes involved in angiogenesis.

Thus, FGF2 and its receptors represent very pertinent targets for therapies aimed at activating or inhibiting angiogenesis processes.

### BRIEF SUMMARY OF THE INVENTION

Provided herein are oligosaccharide compounds corresponding to formula (I):

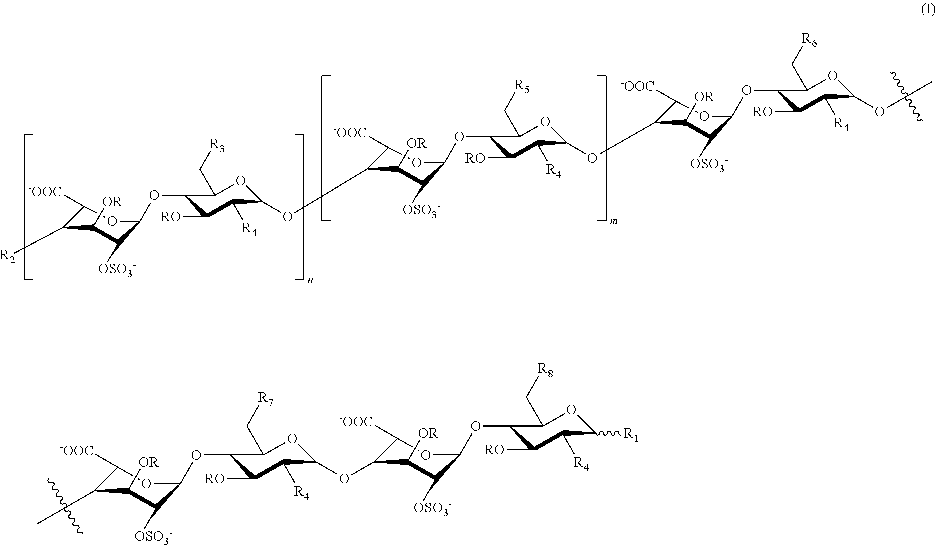

in which:
the wavy line denotes a bond located either below or above the plane of the pyranose ring of the saccharide unit,
R₁ represents an —O-alkyl group, in which said alkyl group contains from 1 to 16 carbon atoms and is optionally substituted with one or more groups, which may be identical or different, chosen from aryl and cycloalkyl groups,
R₂ represents a hydroxyl group or an —O-alkyl group,
R₃, R₅, R₆, R₇ and R₈, which may be identical to or different from one another, represent either an —OSO₃⁻ group or a hydroxyl group,
R₄ represents either an —NH—CO-alkyl group or an —O-alkyl group,
R represents an —O-alkyl group, and
n and m, which may be identical to or different from one another, represent integers equal to 0 or 1,

in acid form or in the form of any one of the pharmaceutically acceptable salts thereof.

### DETAILED DESCRIPTION OF THE INVENTION

We have now found novel synthetic 3-O-alkyl oligosaccharide compounds capable of facilitating the formation of the FGF/FGFR complex and of promoting the formation of new vessels in vitro and in vivo.

A subject of the present invention is novel oligosaccharide compounds corresponding to formula (I):

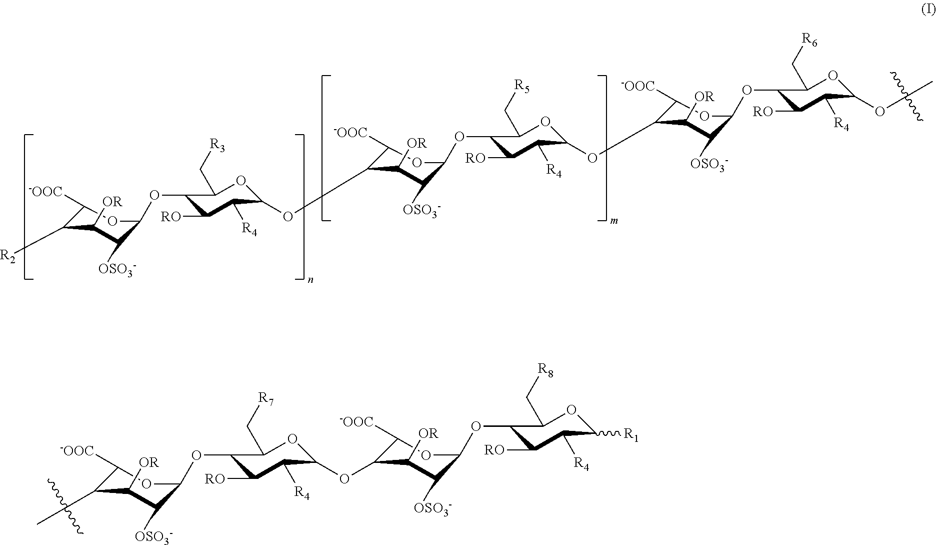

in which:
the wavy line denotes a bond located either below or above the plane of the pyranose ring of the saccharide unit,
R₁ represents an —O-alkyl group, in which said alkyl group contains from 1 to 16 carbon atoms and is optionally substituted with one or more (for example 1 or 2) groups, which may be identical or different, chosen from aryl and cycloalkyl groups,
R₂ represents a hydroxyl group or an —O-alkyl group,
R₃, R₅, R₆, R₇ and R₈, which may be identical to or different from one another, represent either an —OSO₃⁻ group or a hydroxyl group,
R₄ represents either an —NH—CO-alkyl group or an —O-alkyl group,
R represents an —O-alkyl group, and
n and m, which may be identical to or different from one another, represent integers equal to 0 or 1.

In the context of the present invention, and unless otherwise mentioned in the text:
the term “alkyl group” is intended to mean: a linear or branched saturated aliphatic group advantageously comprising between 1 and 6 carbon atoms. By way of examples, mention may be made of methyl, ethyl, propyl, isopropyl, butyl, isobutyl, tert-butyl, pentyl, etc. groups;
the term “cycloalkyl group” is intended to mean: a cyclic alkyl group comprising from 3 to 6 carbon atoms, for example a cyclopentyl or cyclohexyl group;
the term “aryl group” is intended to mean: a cyclic aromatic group comprising between 5 and 10 carbon atoms, such as a phenyl group. Such an aryl group is optionally substituted with one or more groups such as halogen atoms and alkyl, alkoxy, thioalkyl, trifluoromethyl and phenyl groups.

The oligosaccharides according to the invention are synthetic in nature, in the sense that they are compounds obtained by total synthesis from intermediate synthons, as will be described in detail in the text hereinbelow. In this respect, they differ from oligosaccharides obtained by depolymerisation or isolation from complex mixtures of polysaccharides, such as heparins or low-molecular-weight heparins. In particular, the compounds according to the invention have a well-defined structure resulting from their chemical synthesis and are in the form of pure oligosaccharides, i.e. they are free of other oligosaccharide entities.

The invention encompasses the compounds of formula (I) in acid form or in the form of any one of the pharmaceutically acceptable salts thereof. In the acid form, the —COO⁻ and —SO₃⁻ functions are, respectively, in —COOH and —SO₃H form.

The expression “pharmaceutically acceptable salt of the compounds of the invention” is intended to mean a compound in which one or more of the —COO⁻ and/or —SO₃⁻ functions are ionically bonded to a pharmaceutically acceptable cation. The preferred salts according to the invention are those in which the cation is chosen from alkaline metal cations, in particular the Na⁺ cation.

The compounds of formula (I) according to the invention also comprise those in which one or more hydrogen or carbon atoms have been replaced with the radioactive isotope thereof, for example tritium or carbon ¹⁴C. Such labelled compounds are of use in research, metabolism or pharmacokinetic studies, as ligands in biochemical tests.

The oligosaccharides according to the invention stand out from those previously known in that:
the iduronic acids are substituted in position 3 with an alkoxy group, and
the glucosamine units are substituted in position 2 with an acyl group (—NH—CO-alkyl) or with an alkoxy group, and also with an alkoxy group in position 3.

The compounds according to the invention are advantageously octasaccharides, i.e. compounds of formula (I) in which n=1 and m=0 or else n=0 and m=1.

Among the compounds of formula (I) which are subjects of the invention, mention may be made of a subgroup of compounds in which R₁ represents an —O-alkyl group, where said alkyl group contains from 1 to 8 carbon atoms, advantageously from 1 to 5 carbon atoms (for example an —O-methyl or —O-pentyl group), and is optionally substituted with 1 or 2 groups, which may be identical or different, chosen from aryl groups (such as phenyl).

Among the compounds of formula (I) which are subjects of the invention, mention may be made of another subgroup of compounds in which R₂ represents a hydroxyl group or an —O-alkyl group, where said alkyl group comprises from 1 to 4 carbon atoms.

Advantageously, the compounds of formula (I) according to the invention are such that R₂ represents a hydroxyl group.

Among the compounds of formula (I) which are subjects of the invention, mention may be made of another subgroup of compounds in which R₃, R₅, R₆, R₇ and R₈, which may be identical to or different from one another, represent either an —OSO₃⁻ group or a hydroxyl group, on the condition that at least one group among R₃, R₅, R₆, R₇ and R₈ represents an —OSO₃⁻ group.

Another subgroup of compounds of formula (I) is such that at least one of the groups R₃, R₅, R₆, R₇ and R₈ represents an —OSO₃⁻ group and at least one of the groups R₃, R₅, R₆, R₇ and R₈ represents a hydroxyl group.

Another subgroup of compounds of formula (I) is such that R₃, R₅, R₆, R₇ and R₈ all represent —OSO₃⁻ groups.

Another subgroup of compounds of formula (I) is such that R₃, R₅ and R₆ represent —OSO₃⁻ groups and R₇ and R₈ represent hydroxyl groups.

Among the compounds of formula (I) which are subjects of the invention, mention may be made of another subgroup of compounds in which R₄ represents an —NH—CO-alkyl group, where said alkyl group comprises from 1 to 4 carbon atoms, for example a methyl, propyl or isobutyl group.

Among the compounds of formula (I) which are subjects of the invention, mention may be made of another subgroup of compounds in which R₄ represents an —O-alkyl group, where said alkyl group comprises from 1 to 4 carbon atoms, for example a butyl group.

Among the compounds of formula (I) which are subjects of the invention, mention may be made of another subgroup of compounds in which R represents an —O-alkyl group, where said alkyl group comprises from 1 to 4 carbon atoms.

Advantageously, the compounds of formula (I) according to the invention are such that R represents a methoxy group.

Other subgroups of oligosaccharides according to the invention may have several of the characteristics set out above for each of the subgroups previously defined.

Thus, another subgroup of oligosaccharides according to the invention may consist of octasaccharides of formula (I), in which:
n=0 and m=1,
R₁ represents an —O-alkyl group, in which said alkyl group contains from 1 to 5 carbon atoms (for example an —O-methyl or —O-pentyl group), and is optionally substituted with 1 or 2 groups, which may be identical or different, chosen from aryl groups (such as phenyl),
R₂ represents a hydroxyl group or an —O-alkyl group, in which said alkyl group comprises from 1 to 4 carbon atoms,
R₅, R₆, R₇ and R₈, which may be identical to or different from one another, represent either an OSO₃ group or a hydroxyl group, on the condition that at least one of the groups R₅, R₆, R₇ and R₈ represents an —OSO₃⁻ group,
R₄ represents either an —NH—CO-alkyl group or an —O-alkyl group, where said alkyl group comprises from 1 to 4 carbon atoms, and
R represents an —O-alkyl group, in which said alkyl group comprises from 1 to 4 carbon atoms.

Such octasaccharides correspond to formula (I′) below:

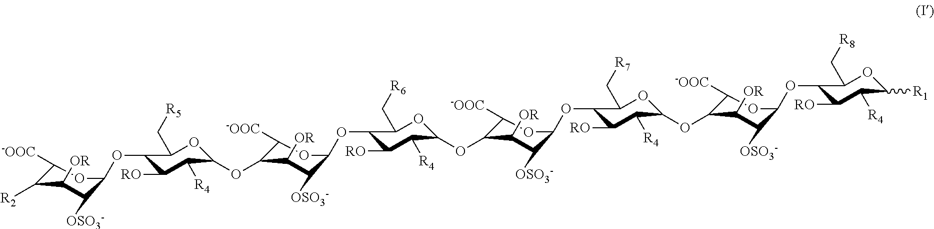

Another subgroup of octasaccharides according to the invention consist of compounds of formula (I), in which:
n=0 and m=1,
R₁ represents an —O-methyl, —O-pentyl or —O-pentylphenyl group,
R₂ represents a hydroxyl group,
R₅, R₆, R₇ and R₈, which may be identical to or different from one another, represent either an —OSO₃⁻ group or a hydroxyl group, on the condition that at least one of the groups R₅, R₆, R₇ and R₈ represents an —OSO₃⁻ group,
R₄ represents either an —NH—CO-alkyl or an —O-alkyl group, where said alkyl group comprises from 1 to 4 carbon atoms, and
R represents an —O-alkyl group, in which said alkyl group comprises from 1 to 4 carbon atoms.

Advantageously, another subgroup of octasaccharides according to the invention consists of compounds of formula (I), in which:
n=0 and m=1,
R₁ represents an —O-methyl, —O-pentyl or —O-pentylphenyl group,
R₂ represents a hydroxyl group,
R₅, R₆, R₇ and R₈, which may be identical to or different from one another, represent either an —OSO₃⁻ group or a hydroxyl group, on the condition that at least one of the groups R₅, R₆, R₇ and R₈ represents an —OSO₃⁻ group,
R₄ is chosen from the groups —NH—CO-methyl, —NH—CO-propyl, —NH—CO-isobutyl and —O-butyl, and
R represents an —O-methyl group.

Among the octasaccharides defined previously, mention may in particular be made of those in which at least one of the groups R₅, R₆, R₇ and R₈ represents an —OSO₃⁻ group and at least one of the groups R₅, R₆, R₇ and R₈ represents a hydroxyl group.

Among the compounds of the invention, mention may in particular be made of the following octasaccharides:
methyl (sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)]₂-(sodium 3-O-methyl-2-O-sodium sulphonato-α-L-idopyranosyluronate)-(1→4)-2-acetamido-2-deoxy-3-O-methyl-6-O-sodium sulphonato-α-D-glucopyranoside (No. 1);
pentyl (sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)]₂-(sodium 3-O-methyl-2-O-sodium sulphonato-α-L-idopyranosyluronate)-(1→4)-2-acetamido-2-deoxy-3-O-methyl-6-O-sodium sulphonato-β-D-glucopyranoside (No. 2);
pentyl (sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)]₂-(sodium 3-O-methyl-2-O-sodium sulphonato-α-L-idopyranosyluronate)-(1→4)-2-butanoylamino-2-deoxy-3-O-methyl-6-O-sodium sulphonato-β-D-glucopyranoside (No. 3);
5-phenylpentyl (sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)]₂-(sodium 3-O-methyl-2-O-sodium sulphonato-α-L-idopyranosyluronate)-(1→4)-2-acetamido-2-deoxy-3-O-methyl-6-O-sodium sulphonato-β-D-glucopyranoside (No. 4);
5-phenylpentyl (sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)]₂-(sodium 3-O-methyl-2-O-sodium sulphonato-α-L-idopyranosyluronate)-(1→4)-2-butanoylamino-2-deoxy-3-O-methyl-6-O-sodium sulphonato-β-D-glucopyranoside (No. 5);
5-phenylpentyl (sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-butanoylamino-2-deoxy-3-O-methyl-α-D-glucopyranoside)-(1→4)-(sodium 3-O-methyl-2-O-sodium sulphonato-α-L-idopyranosyluronate)-(1→4)-2-(butanoylamino)-2-deoxy-3-O-methyl-β-D-glucopyranoside (No. 6);
5-phenylpentyl (sodium 3-O-methyl-2-O-sodium sulphonato-α-L-idopyranosyluronate)-(1→4)-(2-[(3-methylbutanoyl)amino]-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-[(3-methylbutanoyl)amino]-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-[(3-methylbutanoyl)amino]-2-deoxy-3-O-methyl-α-D-glucopyranoside)-(1→4)-(sodium 3-O-methyl-2-O-sodium sulphonato-α-L-idopyranosyluronate)-(1→4)-2-[(3-methylbutanoyl)amino]-2-deoxy-3-O-methyl-β-D-glucopyranoside (No. 7);
5-phenylpentyl (sodium 3-O-methyl-2-O-sodium sulphonato-α-L-idopyranosyluronate)-(1→4)-(2-O-butyl-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-O-butyl-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-O-butyl-3-O-methyl-α-D-glucopyranosyl)-(1→4)-(sodium 3-O-methyl-2-O-sodium sulphonato-α-L-idopyranosyluronate)-(1→4)-2-O-butyl-3-O-methyl-β-D-glucopyranoside (No. 8); and
5-phenylpentyl (sodium 3-O-methyl-2-O-sodium sulphonato-α-L-idopyranosyluronate)-(1→4)-(2-O-butyl-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-O-butyl-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-O-butyl-3-O-methyl-α-D-glucopyranosyl)-(1→4)-(sodium 3-O-methyl-2-O-sodium sulphonato-α-L-idopyranosyluronate)-(1→4)-2-O-butyl-3-O-methyl-α-D-glucopyranoside (No. 9).

In its principle, the process for preparing the compounds according to the invention uses di- or oligosaccharide basic synthons prepared as previously reported in the literature. Reference will be made in particular to the patents or patent applications EP 0 300 099, EP 0 529 715, EP 0 621 282 and EP 0 649 854, and also to the publication by C. Van Boeckel and M. Petitou published in Angew. Chem. Int. Ed. Engl., 1993, 32, 1671-1690. These synthons are then coupled to one another so as to provide an entirely protected equivalent of an oligosaccharide according to the invention. This protected equivalent is then converted into a compound according to the invention. In the coupling reactions mentioned above, a “donor” di- or oligosaccharide, activated on its anomeric carbon, reacts with an “acceptor” di- or oligosaccharide, bearing a free hydroxyl.

The specific synthesis schemes will be described in the detailed examples which follow.

The present invention therefore relates to a process for preparing the oligosaccharides of formula (I), characterized in that:
in a first phase, a fully protected equivalent of the desired oligosaccharide (I) is synthesized, comprising in position 2 of the glucosamine units either an amine function precursor (carbamate or azide for example), or an alkoxy group,
in a second phase, the positions that are to comprise sulphate groups on the final molecule are deprotected and then O-sulphated,
in a third phase, the whole of the compound is deprotected, and
in a fourth phase, if necessary, the N-acyl groups are introduced (introduction of R₄ groups of acyl type).

The synthesis of the fully protected equivalent of the desired oligosaccharide (I) is carried out according to reactions that are well known to those skilled in the art, using methods for the synthesis of oligosaccharides (for example, G. J. Boons, Tetrahedron (1996), 52, 1095-1121 and patent applications WO 98/03554 and WO 99/36443), in which a glycosidic bond-donating oligosaccharide is coupled with a glycosidic bond-accepting oligosaccharide to give another oligosaccharide of which the size is equal to the sum of the sizes of the two reactive entities. This sequence is repeated until the compound of formula (I) is obtained, optionally in protected form. The nature and profile of the charge of the desired final compound determine the nature of the chemical entities used in the various steps of the synthesis, according to the rules well known to those skilled in the art. Reference may be made, for example, to C. Van Boeckel and M. Petitou, Angew. Chem. Int. Ed. Engl. (1993), 32, 1671-1690 or else to H. Paulsen, “Advances in selective chemical syntheses of complex oligosaccharides”, Angew. Chem. Int. Ed. Engl. (1982), 21, 155-173.

The compounds of the invention may naturally be prepared using various strategies known to those skilled in the art of oligosaccharide synthesis. The process described above is the preferred process of the invention. However, the compounds of formula (I) can be prepared via other well-known methods of sugar chemistry, described, for example, in “Monosaccharides, Their chemistry and their roles in natural products”, P. M. Collins and R. J. Ferrier, J. Wiley & Sons (1995) and by G. J. Boons in Tetrahedron (1996), 52, 1095-1121.

The protecting groups, used in the process for preparing the compounds of formula (I), are those that make it possible firstly to protect a reactive function such as a hydroxyl or an amine during a synthesis, and secondly to regenerate the intact reactive function at the end of the synthesis. In the present application, these protecting groups are denoted Pg, Pg′ and Pg″. The protecting groups commonly used in sugar chemistry, as described, for example, in “Protective Groups in Organic Synthesis”, Green et al., 3rd Edition (John Wiley & Sons, Inc., New York), are used to carry out the process according to the invention. The protecting groups are chosen, for example, from acetyl, azide, benzoyl, benzyl, substituted benzyl, benzyl carbamate, isopropylidene, levulinoyl, methyl, tetrahydropyranyl, tert-butyldimethylsilyl (tBDMS) and tert-butyldiphenylsilyl (tBDPS) groups.

Activating groups may also be used; these are those conventionally used in sugar chemistry, for example according to G. J. Boons, Tetrahedron (1996), 52, 1095-1121. These activating groups are chosen, for example, from trichloroacetimidate groups and thioglycosides.

The process described above makes it possible to obtain the compounds of the invention in the form of salts, advantageously in the form of the sodium salt. To obtain the corresponding acids, the compounds of the invention in salt form may be brought into contact with a cation-exchange resin in acidic form. The compounds of the invention in acid form may then be neutralized with a base so as to obtain the desired salt. For the preparation of the salts of the compounds of formula (I), any inorganic or organic base that gives pharmaceutically acceptable salts with the compounds of formula (I) may be used.

A subject of the invention is also the compounds of formula (II) below, in which Alk represents an alkyl group and Pg and Pg′ represent protecting groups as defined previously:

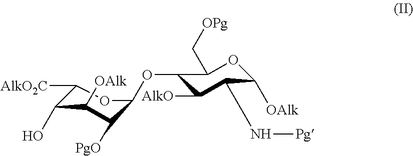

In particular, a subject of the invention is the compound (II) in which the Alk groups represent methyl groups and Pg and Pg′ represent, respectively, acetyl and benzyloxycarbonyl groups (compound 17 in the synthesis schemes which follow).

A subject of the invention is also the compounds of formula (III) below, in which Alk represents an alkyl group, R₁ is as previously defined in relation to the compounds of formula (I), A represents an —NH-Pg″ or —O-alkyl group, and Pg, Pg′ and Pg″, which may be identical to or different from one another, represent protecting groups as previously defined:

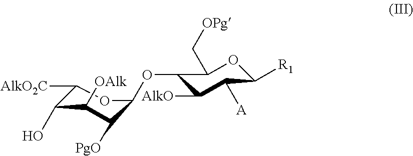

In particular, a subject of the invention is the compounds (III) in which the Alk groups represent methyl groups, R₁ represents an —O-pentyl or —O-pentylphenyl group, Pg represents an acetyl or benzoyl group, Pg′ represents an acetyl or tert-butyldiphenylsilyl group, and A represents an —NH-benzyloxycarbonyl or —O-butyl group.

More particularly, a subject of the invention is the compounds (III) in which:
either Alk represents a methyl group, R₁ represents an —O-pentyl group, Pg and Pg′ represent acetyl groups and A represents an —NH-benzyloxycarbonyl group (compound 44 in the synthesis schemes which follow);
or Alk represents a methyl group, R₁ represents an —O-pentylphenyl group, Pg and Pg′ represent acetyl groups and A represents an —NH-benzyloxycarbonyl group (compound 55 in the synthesis schemes which follow);
or Alk represents a methyl group, R₁ represents an —O-pentylphenyl group, Pg represents an acetyl group, Pg′ represents a tert-butyldiphenylsilyl group and A represents an —NH-benzyloxycarbonyl group (compound 72 in the synthesis schemes which follow);
or Alk represents a methyl group, R₁ represents an —O-pentylphenyl group, Pg represents a benzoyl group, Pg′ represents a tert-butyldiphenylsilyl group and A represents an —O-butyl group (compound 97 in the synthesis schemes which follow).

A subject of the invention is also the compounds of formula (IV) below, in which Alk represents an alkyl group, B represents an azide (N₃) or —O-alkyl group, Pg, Pg′ and Pg″, which may be identical to or different from one another, represent protecting groups as previously defined, and D represents an activating group or an —O-acetyl group:

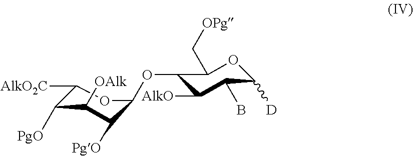

A subject of the invention is also the compounds of formula (IV) above, in which Alk represents an alkyl group, B represents an azide (N₃) or —O-alkyl group, Pg, Pg′ and Pg″, which may be identical to or different from one another, represent protecting groups as previously defined, and D represents an activating group or an —O-acetyl group, with the exception of the compound of formula (IV) in which Alk represents a methyl group, B represents an azide group, Pg represents a levulinyl group, Pg′ and Pg″ represent acetyl groups, and D represents a trichloroacetimidate group.

Advantageously, the compounds of formula (IV) according to the invention are such that B represents an —O-alkyl group.

Advantageously, the compounds of formula (IV) are such that the Alk groups represent methyl groups, B represents an —O-butyl group, Pg represents a benzyl or levulinyl group, Pg′ represents an acetyl or benzoyl group, Pg″ represents an acetyl or tert-butyldiphenylsilyl group and D represents an activating group such as the trichloroacetimidate (—O—C(NH)CCl₃) group or an —O-acetyl group.

In particular, a subject of the invention is the compounds (IV) in which the Alk groups represent methyl groups, B represents an azide (N₃) group, Pg represents a benzyl or levulinyl group, Pg′ represents an acetyl or benzoyl group, Pg″ represents an acetyl or tert-butyldiphenylsilyl group and D represents an activating group such as the trichloroacetimidate (—O—C(NH)CCl₃) group or an —O-acetyl group, with the exception of the compound of formula (IV) in which Alk represents a methyl group, B represents an azide group, Pg represents a levulinyl group, Pg′ and Pg″ represent acetyl groups, and D represents a trichloroacetimidate group.

More particularly, a subject of the invention is the compounds (IV) in which:
either Alk represents a methyl group, B represents an azide (N₃) group, Pg represents a benzyl group, Pg′ and Pg″ represent acetyl groups, and D represents an —O—C(NH)CCl₃ group (compound 28 in the synthesis schemes which follow);
or Alk represents a methyl group, B represents an azide (N₃) group, Pg represents a levulinyl group, Pg′ and Pg″ represent acetyl groups, and D represents an —O—C(NH)CCl₃ group (compound 29 in the synthesis schemes which follow);
or Alk represents a methyl group, B represents an azide (N₃) group, Pg represents a levulinyl group, Pg′ represents an acetyl group, Pg″ represents a tert-butyldiphenylsilyl group, and D represents an —O—C(NH)CCl₃ group (compound 69 in the synthesis schemes which follow);
or Alk represents a methyl group, B represents an —O-butyl group, Pg represents a levulinyl group, Pg′ represents a benzoyl group, Pg″ represents an acetyl group, and D represents an —O-acetyl group (compound 91 in the synthesis schemes which follow);
or Alk represents a methyl group, B represents an —O-butyl group, Pg represents a levulinyl group, Pg′ represents a benzoyl group, Pg″ represents an acetyl group, and D represents an —O—C(NH)CCl₃ group (compound 93 in the synthesis schemes which follow);
or Alk represents a methyl group, B represents an —O-butyl group, Pg represents a levulinyl group, Pg′ represents a benzoyl group, Pg″ represents a tert-butyldiphenylsilyl group, and D represents an —O—C(NH)CCl₃ group (compound 101 in the synthesis schemes which follow).

Such compounds of formulae (II), (III) and (IV) are of use as synthesis intermediates for the compounds of formula (I).

### EXEMPLIFICATION

The examples which follow describe the preparation of certain compounds in accordance with the invention. These examples are not limiting, and merely illustrate the present invention. The starting compounds and the reagents, when their mode of preparation is not expressly described, are commercially available or described in the literature, or else can be prepared according to methods which are described therein or which are known to those skilled in the art.

The following abbreviations are used:
[α]_{D}: optical rotation
Ac: acetyl
All: allyl
Bn: benzyl
BT: benzotriazole
Bz: benzoyl
TLC: Thin Layer Chromatography
DDQ: 2,3-dichloro-5,6-dicyano-1,4-benzoquinone
CE⁽¹⁾: Capillary Electrophoresis
ESI: Electron Spray Ionization
ESI-MS⁽²⁾: ESI coupled to Mass Spectrometry
Et: ethyl
h: hours
LC-MS⁽³⁾: Liquid Chromatography coupled to Mass Spectrometry
Lev: levulinyl
Me: methyl
min: minute(s)
mL: millilitre(s)
mmol: millimol(s)
p: para
Phe: phenyl
Pent: pentyl
Rf: Retardation factor (retention time measured on TLC relative to the solvent migration front)
NMR: Nuclear Magnetic Resonance
SFC: Supercritical Fluid Chromatography
SFC-MS⁽⁴⁾: SFC coupled to Mass Spectrometry
tert: tertiary
TBDMS: tert-butyldimethylsilyl
TBDPS: tert-butyldiphenylsilyl
THP: tetrahydropyran
Z: benzyloxycarbonyl ⁽¹⁾The capillary electrophoresis operations are carried out using a Beckman apparatus under the following conditions: capillary: PVA Coated 40 cm (Ldet)×50 μm (id), electrolyte: 4 mM 5-sulphosalicylic acid, pH 3.51 (NaOH), detection: 214 nm indirect, voltage: −15 kV, T°=30° C., injection: 5 sec (0.5 psi), solution 0.5 mg/ml, coinjection: 5 sec (0.5 psi) DMSO.⁽²⁾The ESI-MS spectra are recorded using an LCT apparatus (Waters) with a TOF (Time-Of-Flight) analyser. The introduction mode is direct by infusion, the ionization mode is by positive-mode or negative-mode electrospray as appropriate.⁽³⁾The LC-MS are performed on a Waters ZQ4000 apparatus. The column used is a Symmetry C18 3.5 μm (2.1×50 mm) column. Eluent A is made up of H₂O+0.005% TFA, pH 3.15. Eluent B is made of acetonitrile+0.005% TFA. The gradient ranges from 0 to 90% of eluent B over 10 (or 30) min+5 min at 90% of eluent B. The flow rate is 0.4 ml/min.⁽⁴⁾The SFC-MS are carried out with a Mettler Toledo apparatus using a Diol 60A 5 μm column (250×4.6 mm)−T°=34° C.—gas: CO₂—modifier: 50% MeOH/50% CH₃CN—flow rate: 3 ml/min—pressure: 180 bar—gradient: 5% (2 min), 3%/min 35% (1 min) 95%/min 5%. Run time: 16 min. Mass spectrometry: positive electrospray.

Preparation of the Synthesis Intermediates:

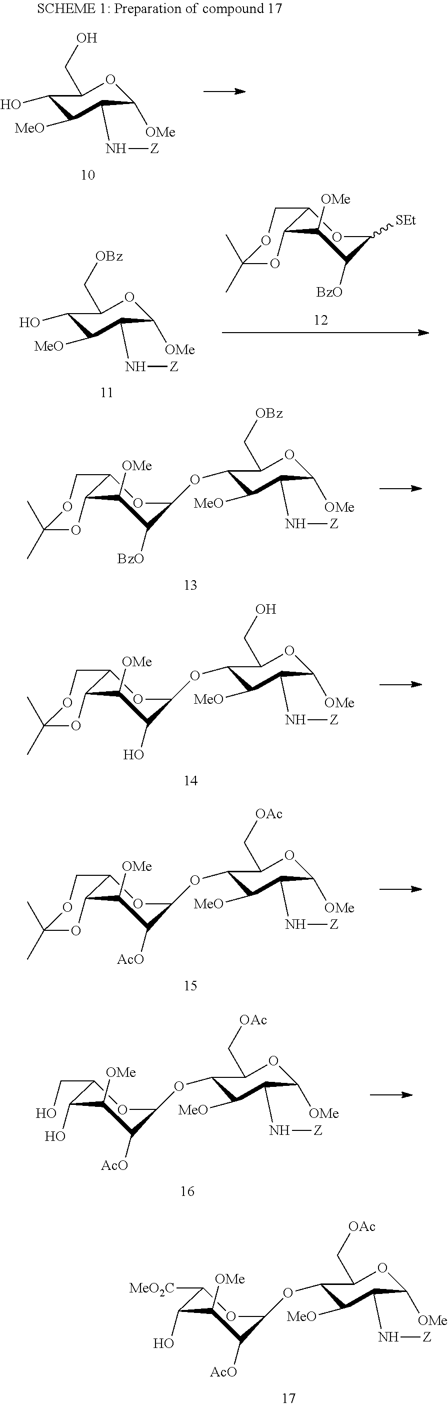

### Methyl 6-O-benzoyl-2-[(benzyloxy)carbonyl]amino-2-deoxy-3-O-methyl-α-D-glucopyranoside (11)

Triethylamine (8.3 ml, 59.9 mmol) and then BzOBt (13.6 g, 56.7 mmol) are added, at ambient temperature, to a solution of methyl 2-[(benzyloxy)carbonyl]amino-2-deoxy-3-O-methyl-α-D-glucopyranoside (10) (5.38 g, 15.8 mmol; described by Akiya, Shichiro and Osawa, Toshiaki in Yakugaku Zasshi, 1956, 76, 1276-9) in dichloromethane (240 ml). After stirring at ambient temperature for 16 hours, the mixture is diluted with dichloromethane (800 ml). The organic phase is washed with a 2% aqueous solution of sodium hydrogen carbonate and then with water, dried over sodium sulphate, filtered, and then concentrated to dryness. Purification of the residue by flash chromatography on a silica gel column (3/2 v/v dichloromethane/ethyl acetate) gives 5.65 g of compound 11.

Rf=0.42, silica gel, 5/1 v/v dichloromethane/ethyl acetate

### Methyl (2-O-benzoyl-4,6-O-isopropylidene-3-O-methyl-α-L-idopyranosyl)-(1→4)-6-O-benzoyl-2-[(benzyloxy)carbonyl]amino-2-deoxy-3-O-methyl-α-D-glucopyranoside (13)

A mixture of ethyl 2-O-benzoyl-4,6-O-isopropylidene-3-O-methyl-1-thio-α-L-idopyranoside 12 (3.61 g, 9.44 mmol; prepared according to Jaurand, G. et al. Bioorg. Med. Chem. Lett. 1992, 2, 897-900), of compound 11 (3.50 g, 7.86 mmol) and of 4 Å molecular sieve powder (1.90 g) in dichloromethane (62 ml) is stirred under an argon atmosphere for 1 h. The mixture is then cooled to 0° C. and N-bromosuccinimide (4.03 g, 22.7 mmol) and trifluoromethanesulphonic acid (182 μL, 2.08 mmol) are successively added. After magnetic stirring for 45 min, sodium hydrogen carbonate is added and the reaction mixture is then filtered and diluted with dichloromethane (450 ml). The organic phase is washed with a 1M aqueous solution of sodium thiosulphate and then with water, dried over sodium sulphate, filtered, and then concentrated to dryness. Purification of the residue by flash chromatography on a silica gel column (1/1 v/v cyclohexane/ethyl acetate) gives 5.66 g of compound 13.

Rf=0.5, silica gel, 7/5 v/v cyclohexane/ethyl acetate

### Methyl (4,6-O-isopropylidene-3-O-methyl-α-L-idopyranosyl)-(1→4)-2-[(benzyloxy)carbonyl]amino-2-deoxy-3-O-methyl-α-D-glucopyranoside (14)

Potassium tert-butoxide (829 mg, 7.38 mmol) is added to a solution of compound 13 (5.65 g, 7.38 mmol) in a methanol-dioxane mixture (74 ml, 1/1, v/v). The reaction mixture is then stirred for 2 h at ambient temperature and then neutralized with Dowex AG50WX4 resin, filtered, and then concentrated to dryness. The residue obtained is purified by flash chromatography on a silica gel column (2/3 v/v dichloromethane/acetone), to give 3.63 g of compound 14.

Rf=0.56, silica gel, 2/1 v/v dichloromethane/acetone

### Methyl (2-O-acetyl-4,6-O-isopropylidene-3-O-methyl-α-L-idopyranosyl)-(1→4)-6-O-acetyl-2-[(benzyloxy)carbonyl]amino-2-deoxy-3-O-methyl-α-D-glucopyranoside (15)

Compound 14 (3.62 g, 6.51 mmol) is dissolved in dichloromethane (26 ml), and then triethylamine (2.7 ml, 19.5 mmol), 4-dimethylaminopyridine (80 mg, 0.65 mmol) and acetic anhydride (1.8 ml, 18.2 mmol) are added. After stirring at 0° C. for 10 minutes, and then at ambient temperature for 2 h, the reaction mixture is diluted with dichloromethane (500 ml) and then successively washed with a 10% aqueous solution of potassium hydrogen sulphate, with water and with a 2% aqueous solution of sodium hydrogen carbonate, and the organic phase is then dried over sodium sulphate, filtered and concentrated. The resulting residue is purified by flash chromatography on a silica gel column (3/7 v/v cyclohexane/ethyl acetate), to give 4.19 g of compound 15.

Rf=0.48, silica gel, 5/7 v/v cyclohexane/ethyl acetate

### Methyl (2-O-acetyl-3-O-methyl-α-L-idopyranosyl)-(1→4)-6-O-acetyl-2-[(benzyloxy)carbonyl]amino-2-deoxy-3-O-methyl-α-D-glucopyranoside (16)

Compound 15 (4.18 g, 6.52 mmol) is dissolved in acetic acid (65 ml). The reaction medium is stirred at ambient temperature for 16 h. After concentration under vacuum and codistillation with toluene (4×100 ml), the resulting residue is purified by flash chromatography on a silica gel column (1/4 v/v cyclohexane/acetone), to give 3.62 g of compound 16.

Rf=0.47, silica gel, 2/3 v/v cyclohexane/acetone

### Methyl (methyl 2-O-acetyl-3-O-methyl-α-L-idopyranosyluronate)-(1→4)-6-O-acetyl-2-[(benzyloxy)carbonyl]amino-2-deoxy-3-O-methyl-α-D-glucopyranoside (17)

A saturated aqueous solution of sodium hydrogen carbonate (24 ml) is added to a solution of compound 16 (3.62 g, 6.02 mmol) in tetrahydrofuran (80 ml), and then, at 0° C. and under argon, a 0.32 M solution of 2,2,6,6-tetramethylpiperidin-1-oxy (376 μl, 0.12 mmol) and a solution of 1,3-dibromo-5,5-dimethylhydantoin (10.3 ml, 12 mmol) are successively added. After stirring at ambient temperature for 4 h 30, the reaction medium is concentrated and then coevaporated with N,N-dimethylformamide (4×50 ml). The residue obtained is placed in solution in N,N-dimethylformamide (80 ml) and potassium hydrogen carbonate (3.01 g, 30.1 mmol) and then methyl iodide (3.7 ml, 60.2 mmol) are added at 0° C. and under argon. After completion of the reaction (TLC), the reaction medium is concentrated under vacuum, and the reaction crude is diluted with ethyl acetate (800 ml), washed with water and then with a 1M aqueous solution of sodium thiosulphate, dried over sodium sulphate, filtered and concentrated. The resulting residue is purified by chromatography on a Sephadex® LH20 column (190×3.2 cm, 1/1 dichloromethane/ethanol) followed by flash chromatography on a silica gel column (2/3 v/v cyclohexane/acetone) to give 3.90 g of compound 17.

Rf=0.36, silica gel, 1/1 v/v cyclohexane/acetone

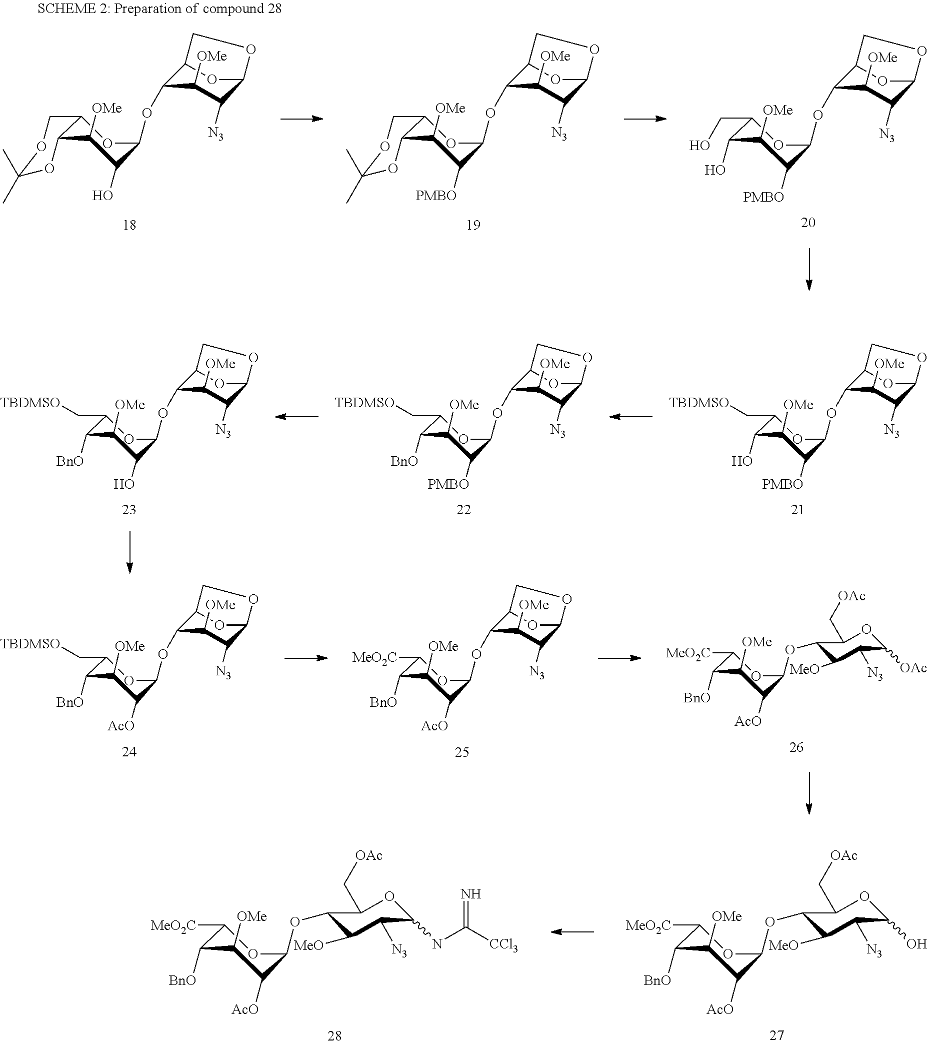

### (4,6-O-Isopropylidene-2-O-p-methoxybenzyl-3-O-methyl-α-L-idopyranosyl)-(1→4)-1,6-anhydro-2-azido-2-deoxy-3-O-methyl-β-D-glucopyranose (19)

p-methoxybenzyl chloride (1.3 ml, 9.48 mmol) and then 55% sodium hydride (370 mg, 7.70 mmol) are added, at 0° C. and under argon, to a solution of compound 18 (2.47 g, 5.92 mmol; WO2010/029185) in N,N-dimethylformamide (24 ml). After stirring for 16 h, methanol is added, the reaction medium is concentrated under vacuum, and the residue is diluted with ethyl acetate (500 ml), washed with water, dried over sodium sulphate, filtered and concentrated. The residue obtained is purified by flash chromatography on silica gel (55/45 v/v cyclohexane/acetone), to give 3.18 g of compound 19.

Rf=0.42, silica gel, 3/2 v/v cyclohexane/acetone

### (2-O-p-Methoxybenzyl-3-O-methyl-α-L-idopyranosyl)-(1→4)-1,6-anhydro-2-azido-2-deoxy-3-O-methyl-β-D-glucopyranose (20)

Compound 19 (3.17 g, 5.91 mmol) is dissolved in acetic acid (60 ml). The reaction medium is stirred at ambient temperature for 16 h. After concentration under vacuum and codistillation with toluene (4×100 ml), the residue obtained is purified by flash chromatography on a silica gel column (3/7 v/v cyclohexane/acetone), to give 2.66 g of compound 20.

Rf=0.45, silica gel, 1/1 v/v cyclohexane/acetone

### (6-O-tert-Butyldimethylsilyl-2-O-p-methoxybenzyl-3-O-methyl-α-L-idopyranosyl)-(1→4)-1,6-anhydro-2-azido-2-deoxy-3-O-methyl-β-D-glucopyranose (21)

Compound 20 (2.67 g, 5.34 mmol) is dissolved in dichloromethane (53 ml), and then triethylamine (1.6 ml, 11.7 mmol), 4-dimethylaminopyridine (65 mg, 0.53 mmol) and tert-butyldimethylsilyl chloride (886 mg, 5.87 mmol) are added. After stirring at 0° C. for 30 minutes, and then at ambient temperature for 5 h, the same amount of reactants is added. After stirring at ambient temperature for 16 h, the reaction mixture is diluted with dichloromethane (500 ml), and then successively washed with a 10% aqueous solution of potassium hydrogen sulphate and with water and then the organic phase is dried over sodium sulphate, filtered and concentrated. The resulting residue is purified by flash chromatography on a silica gel column (7/3 v/v cyclohexane/acetone), to give 3.46 g of compound 21.

Rf=0.50, silica gel, 2/1 v/v cyclohexane/acetone

### (4-O-Benzyl-6-O-tert-butyldimethylsilyl-2-O-p-methoxybenzyl-3-O-methyl-α-L-idopyranosyl)-(1→4)-1,6-anhydro-2-azido-2-deoxy-3-O-methyl-β-D-glucopyranose (22)

Benzyl bromide (1.3 ml, 26.7 mmol) and then 55% sodium hydride (385 mg, 8.01 mmol) are added, at 0° C. and under argon, to a solution of compound 21 (3.26 g, 5.34 mmol) in N,N-dimethylformamide (27 ml). After stirring for 3 h, methanol (3 ml) is added, the reaction medium is concentrated under vacuum, and the residue is diluted with ethyl acetate (500 ml), washed with water, dried over sodium sulphate, filtered and concentrated under vacuum. The residue then obtained is purified by flash chromatography on a silica gel column (7/3 v/v cyclohexane/acetone), to give 3.67 g of compound 22.

Rf=0.54, silica gel, 5/2 v/v cyclohexane/acetone

### (4-O-Benzyl-6-O-tert-butyldimethylsilyl-3-O-methyl-α-L-idopyranosyl)-(1→4)-1,6-anhydro-2-azido-2-deoxy-3-O-methyl-β-D-glucopyranose (23)

Water (10 ml) and then, at 0° C., DDQ (1.78 g, 7.85 mmol) are added to a solution of compound 22 (3.67 g, 5.23 mmol) in dichloromethane (210 ml). After stirring at 0° C. for 5 h 30, the medium is diluted with dichloromethane (700 ml) and a 2% aqueous solution of sodium hydrogen carbonate is added. The organic phase is then washed with water, dried over sodium sulphate, filtered and concentrated. The residue obtained is purified by flash chromatography on a silica gel column (7/3 v/v toluene/ethyl acetate), to give 2.87 g of compound 23.

Rf=0.45, silica gel, 2/1 v/v toluene/acetone

### (2-O-Acetyl-4-O-benzyl-6-O-tert-butyldimethylsilyl-3-O-methyl-α-L-idopyranosyl)-(1→4)-1,6-anhydro-2-azido-2-deoxy-3-O-methyl-β-D-glucopyranose (24)

Compound 23 (2.86 g, 4.92 mmol) is dissolved in dichloromethane (20 ml), and then triethylamine (1.0 ml, 7.37 mmol), 4-dimethylaminopyridine (60 mg, 0.50 mmol) and acetic anhydride (650 μl, 6.88 mmol) are added. After stirring at 0° C. for 1 h and then at ambient temperature for 16 h, the reaction mixture is diluted with dichloromethane (50 ml), and then successively washed with a 10% aqueous solution of potassium hydrogen sulphate and with water, and then the organic phase is dried over sodium sulphate, filtered and concentrated. The resulting residue is purified by flash chromatography on a silica gel column (7/3 v/v toluene/ethyl acetate), to give 3.46 g of compound 24.

Rf=0.6, silica gel, 2/1 v/v toluene/ethyl acetate

### (Methyl 2-O-acetyl-4-O-benzyl-3-O-methyl-α-L-idopyranosyluronate)-(1→4)-1,6-anhydro-2-azido-2-deoxy-3-O-methyl-β-D-glucopyranose (25)

A solution of chromium trioxide (1.2 g) in 3.5M sulphuric acid (5.4 ml) is added, at 0° C., to a solution of compound 24 (3.0 g, 4.83 mmol) in acetone (193 ml). After stirring at 0° C. for 5 h 30, the reaction medium is diluted with dichloromethane (800 ml), washed with water, dried over sodium sulphate, filtered and concentrated. The compound obtained is used in the next step without purification. The residue obtained is dissolved in N,N-dimethylformamide (63 ml), and potassium hydrogen carbonate (2.42 g, 24.1 mmol) and also methyl iodide (3.0 ml, 48.3 mmol) are added at 0° C. The reaction mixture is stirred at ambient temperature for 4 h, and then concentrated under vacuum. The residue is diluted with ethyl acetate (800 ml) and then washed with water, with a saturated aqueous solution of sodium thiosulphate and with a saturated aqueous solution of sodium chloride, and then dried over sodium sulphate, filtered and concentrated. The resulting residue is purified by flash chromatography on a silica gel column (3/2 v/v toluene/ethyl acetate), to give 2.08 g of compound 25.

Rf=0.47, silica gel, 1/1 v/v toluene/ethyl acetate

### (Methyl 2-O-acetyl-4-O-benzyl-3-O-methyl-α-L-idopyranosyluronate)-(1→4)-1,6-di-O-acetyl-2-azido-2-deoxy-3-O-methyl-α,β-D-glucopyranose (26)

Trifluoroacetic acid (923 μl, 12 mmol) is added, at 0° C., to a solution of compound 25 (585 mg, 1.09 mmol) in acetic anhydride (10.3 ml). The reaction medium is stirred for 4 h at ambient temperature. After concentration under vacuum, the mixture is coevaporated with toluene. Purification of the residue by chromatography on a silica gel column (toluene/acetone) gives 694.5 mg of compound 26.

¹H NMR [500 MHz] (CDCl₃) δ of the anomeric protons: Glc^{I} β 5.42 ppm, Glc^{I} α 6.17 ppm and IdoUA^{II} 5.1 ppm.

### (Methyl 2-O-acetyl-4-O-benzyl-3-O-methyl-α-L-idopyranosyluronate)-(1→4)-6-O-acetyl-2-azido-2-deoxy-3-O-methyl-α,β-D-glucopyranose (27)

Benzylamine (4.5 ml, 41.2 mmol) is added, under an argon atmosphere at 0° C., to a solution of compound 26 (694 mg, 1.09 mmol) in diethyl ether (32 ml). The reaction medium is stirred for 3 h at ambient temperature and then stored at +4° C. for 21 h. After dilution with ethyl acetate, the reaction medium is successively washed with an aqueous solution of hydrochloric acid (1M) and then with water. The organic phase is dried over sodium sulphate, filtered and concentrated under vacuum. The resulting residue is purified by chromatography on a silica gel column, to give 578.1 mg of compound 27.

¹H NMR [500 MHz] (CDCl₃) δ of the anomeric protons: Glc^{I} β 4.56 ppm, Glc^{I} α 5.26 ppm and IdoUA^{II} 5.12 ppm.

### (Methyl 2-O-acetyl-4-O-benzyl-3-O-methyl-α-L-idopyranosyluronate)-(1→4)-6-O-acétyl-2-azido-2-deoxy-3-O-methyl-α,β-D-glucopyranose trichloroacetimidate (28)

Trichloroacetonitrile (476 μl, 4.74 mmol) and caesium carbonate (469 mg, 1.44 mmol) are added at 0° C. to a solution of compound 27 (566.9 mg, 0.95 mmol) in dichloromethane (19 ml) in the presence of 4 Å molecular sieve powder (950 mg). After stirring at ambient temperature for 16 h, the reaction medium is filtered through Celite® and then concentrated. The residue is purified by chromatography on a silica gel column, to give 608 mg of compound 28.

¹H NMR [500 MHz] (CDCl₃) δ of the anomeric protons: Glc^{I} β 5.60 ppm, Glc^{I} α 6.30 ppm and IdoUA^{II} 5.13 ppm.

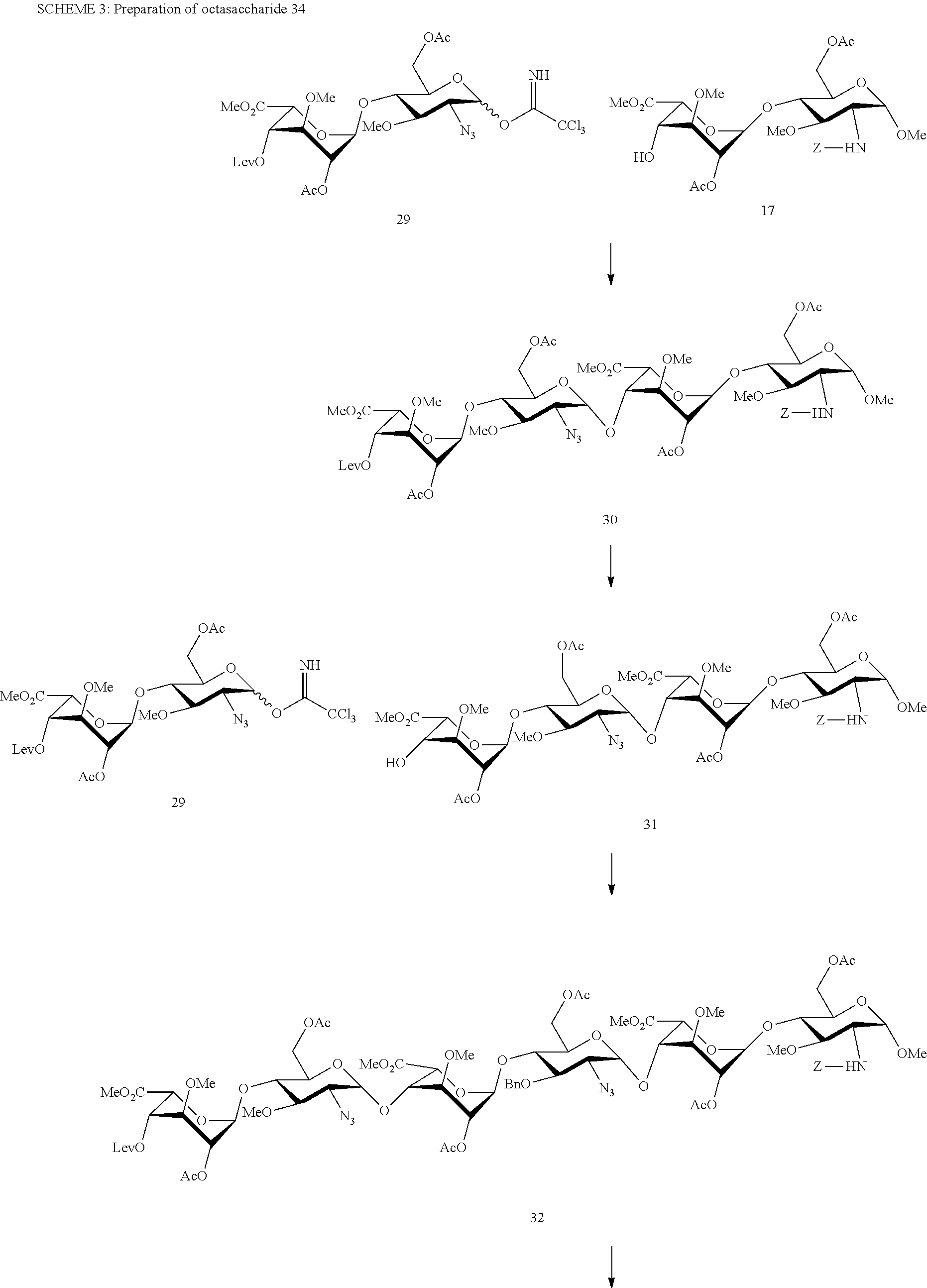

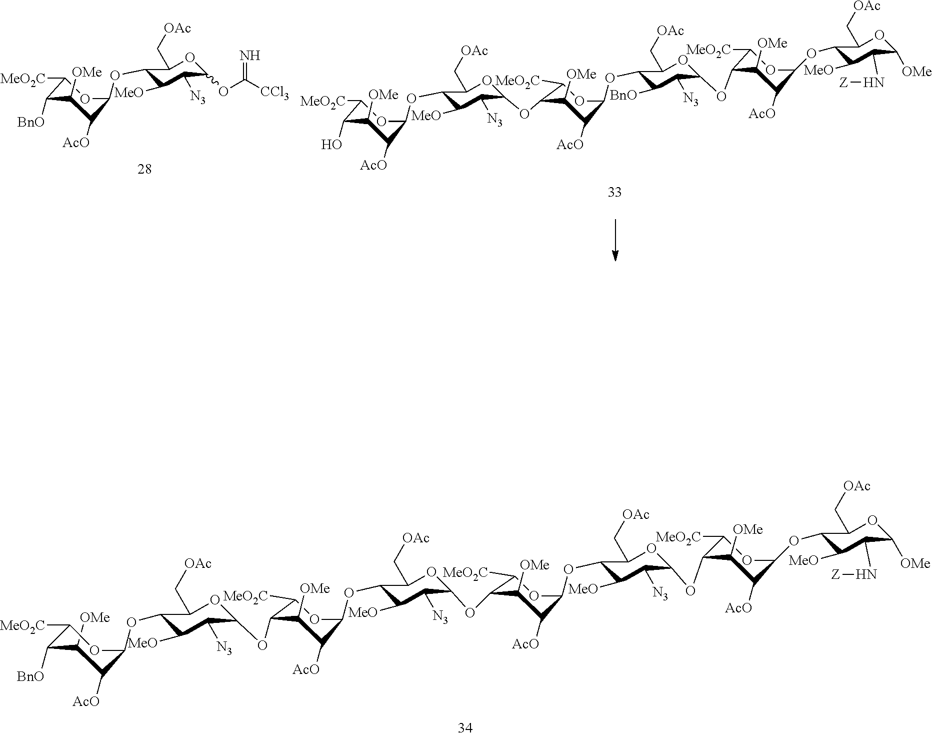

### Methyl (methyl 2-O-acetyl-4-O-levulinoyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-6-O-acetyl-2-[(benzyloxy)carbonyl]amino-2-deoxy-3-O-methyl-α-D-glucopyranoside (30)

A mixture of compound 29 (363 mg, 0.49 mmol) (described in WO2010/029185), of the glycosyl acceptor 17 (489 mg, 0.77 mmol) and of 4 Å molecular sieve powder (363 mg) in dichloromethane (40 ml) is stirred under an argon atmosphere for 1 h at 25° C. The reaction mixture is cooled to −25° C. and a 1M solution of tert-butyldimethylsilyl triflate in dichloromethane (73 μl) is added to the reaction medium. After stirring for 15 minutes, the reaction medium is neutralized by adding solid sodium hydrogen carbonate. After filtration and concentration, the organic phase is washed with a 2% aqueous solution of sodium hydrogen carbonate and with water, dried over sodium sulphate, filtered and then concentrated to dryness. The residue obtained is purified by size exclusion chromatography (Sephadex® LH20, 190×3.2 cm, 1/1 v/v dichloromethane/ethanol), to give 393 mg of compound 30.

¹H NMR [500 MHz] (CDCl₃) δ of the anomeric protons Glc^{I} α 4.64 ppm, IdoUA^{II} α 5.07 ppm, Glc^{III} α 4.97 ppm and IdoUA^{IV} α 5.05 ppm.

### Methyl (methyl 2-O-acetyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-6-O-acetyl-2-[(benzyloxy)carbonyl]amino-2-deoxy-3-O-methyl-α-D-glucopyranoside (31)

Hydrazine acetate (253 mg, 2.75 mmol) is added to a solution of compound 30 (670 mg, 0.55 mmol) in a 1/2 v/v toluene/ethanol mixture (290 ml). The reaction medium is stirred for 1 h at ambient temperature. After concentration, the residue is purified by flash chromatography on a silica gel column (1/9 v/v toluene/ethyl acetate), to give 677 mg of compound 31.

¹H NMR [500 MHz] (CDCl₃) δ of the anomeric protons Glc^{I} α 4.64 ppm, IdoUA^{II} α 5.08 ppm, Glc^{III} α 4.98 ppm and IdoUA^{IV} α 4.98 ppm.

### Methyl (methyl 2-O-acetyl-4-O-levulinoyl-3-O-methyl-α-L-idopyranosyluronate)-(1→4)-[(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)]₂-6-O-acetyl-2-[(benzyloxy)carbonyl]amino-2-deoxy-3-O-methyl-α-D-glucopyranoside (32)

A mixture of compound 29 (442 mg, 0.59 mmol), of the glycosyl acceptor 31 (677 mg, 0.60 mmol) and of 4 Å molecular sieve powder (442 mg) in dichloromethane (21 ml) is stirred under an argon atmosphere for 1 h at 25° C. The reaction mixture is cooled to −25° C. and a 1M solution of tert-butyldimethylsilyl triflate in dichloromethane (90 μl) is added to the reaction medium. After stirring for 15 minutes, the reaction medium is neutralized by adding solid sodium hydrogen carbonate. After filtration and concentration, the organic phase is washed with a 2% aqueous solution of sodium hydrogen carbonate and with water, dried over sodium sulphate, filtered and then concentrated to dryness. The residue obtained is purified by size exclusion chromatography (Sephadex® LH20, 190×3.2 cm, 1/1 v/v dichloromethane/ethanol), to give 564 mg of compound 32.

¹H NMR [500 MHz] (CDCl₃) δ of the anomeric protons Glc^{I} α 4.64 ppm, IdoUA^{II} α 5.08 ppm, Glc^{III} α 5.0 ppm, IdoUA^{IV} α 5.08 ppm, Glc^{V} α 4.98 ppm and IdoUA^{VI} α 5.07 ppm.

### Methyl (methyl 2-O-acetyl-3-O-methyl-α-L-idopyranosyluronate)-(1→4)-[(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)]₂-6-O-acetyl-2-[(benzyloxy)carbonyl]amino-2-deoxy-3-O-methyl-α-D-glucopyranoside (33)

Hydrazine acetate (152 mg, 1.65 mmol) is added to a solution of compound 32 (564 mg, 0.33 mmol) in a 1/2 v/v toluene/ethanol mixture (66 ml). The reaction medium is stirred at ambient temperature for 1 h. After concentration, the residue is purified by flash chromatography on a silica gel column (1/9 v/v toluene/ethyl acetate), to give 480 mg of compound 33.

Rf=0.49, silica gel, 1/9 v/v toluene/ethyl acetate.

### Methyl (methyl 2-O-acetyl-4-O-benzyl-3-O-methyl-α-L-idopyranosyluronate)-(1→4)-[(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)]₃-6-O-acetyl-2-[(benzyloxy)carbonyl]amino-2-deoxy-3-O-methyl-α-D-glucopyranoside (34)

A mixture of compound 28 (332 mg, 0.447 mmol), of the glycosyl acceptor 33 (480 mg, 0.298 mmol) and of 4 Å molecular sieve powder (224 mg) in dichloromethane (11 ml) is stirred under an argon atmosphere for 1 h at ambient temperature. The reaction mixture is cooled to −20° C. and a 0.1M solution of tert-butyldimethylsilyl triflate in dichloromethane (4.5 ml) is added to the reaction medium. After 1 h 30 min, the reaction medium is neutralized by adding solid sodium hydrogen carbonate. After filtration and concentration under vacuum, the organic phase is washed with a 2% aqueous solution of sodium hydrogen carbonate and with water, dried over sodium sulphate, filtered and then concentrated to dryness. The residue obtained is purified by chromatography on a silica gel column, to give 500 mg of compound 34.

¹H NMR [500 MHz] (CDCl₃) δ of the anomeric protons Glc^{I} α 4.65 ppm, IdoUA^{II} α 5.10 ppm, Glc^{III} α 4.97 ppm, IdoUA^{IV} α 5.10 ppm, Glc^{V} α 4.98 ppm, IdoUA^{VI} α 5.10 ppm, Glc^{VII} α 5.00 ppm and IdoUA^{VIII} α 5.12 ppm.

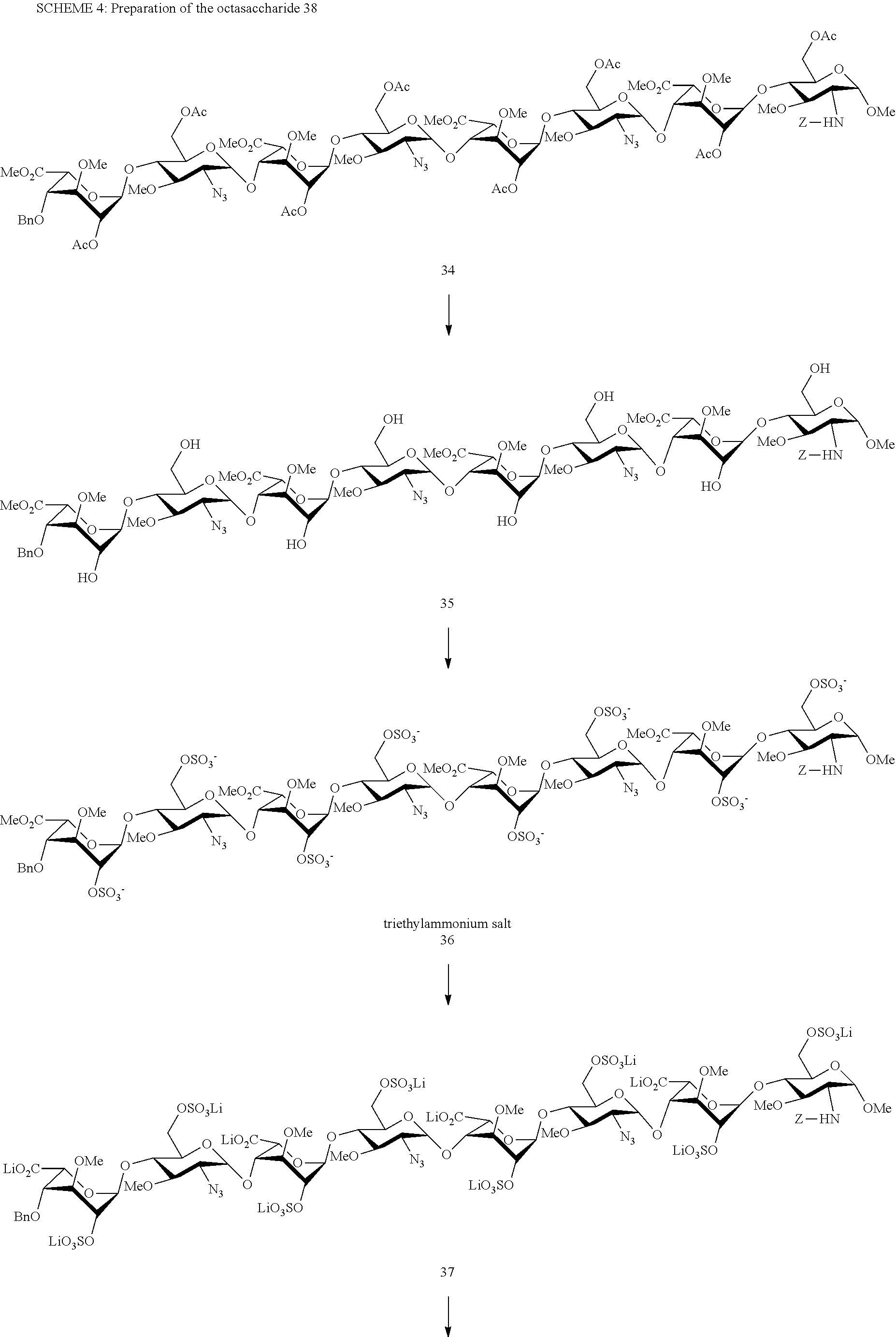

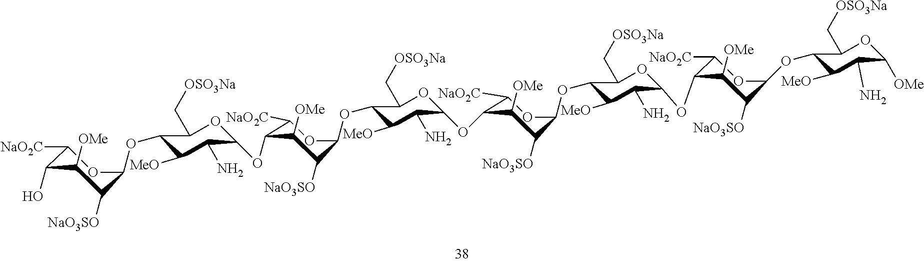

### Methyl (methyl 4-O-benzyl-3-O-methyl-α-L-idopyranosyluronate)-(1→4)-[(2-azido-2-deoxy-3-O-methyl-α-D-glucopyranosyl)-(1→4)-(methyl 3-O-methyl-α-L-idopyranosyluronate)-(1→4)]₃-2-[(benzyloxy)carbonyl]amino-2-deoxy-3-O-methyl-α-D-glucopyranoside (35)

A 1M solution of sodium methoxide in methanol (684 μl) is added, at 0° C., under an argon atmosphere, to a solution of compound 34 (500 mg, 0.228 mmol) in a 2/3 v/v dichloromethane/methanol mixture (68 ml) containing 3 Å molecular sieve (285 mg). After magnetic stirring at ambient temperature for 18 h, the reaction medium is neutralized with Dowex® 50WX4 H⁺ resin. After filtration and concentration under vacuum, the residue is purified by chromatography on a silica gel column, to give 170 mg of compound 35.

Rf=0.54, silica gel, 9/1 v/v dichloromethane/methanol.

### Methyl (methyl 4-O-benzyl-3-O-methyl-2-O-triethylammonium sulphonato-α-L-idopyranosyluronate)-(1→4)-[(2-azido-2-deoxy-3-O-methyl-6-O-triethylammonium sulphonato-α-D-glucopyranosyl)-(1→4)-(methyl 3-O-methyl-2-O-triethylammonium sulphonato-α-L-idopyranosyluronate)-(1→4)]₃-2-[(benzyloxy)carbonyl]amino-2-deoxy-3-O-methyl-6-O-triethylammonium sulphonato-α-D-glucopyranoside (36)

Compound 35 (84.5 mg, 0.046 mmol) is dried by codistillation of anhydrous N,N-dimethylformamide (3×4 ml) and is then placed in solution in anhydrous N,N-dimethylformamide (4 ml). The sulphur trioxide-trithylamine complex (331 mg, 1.825 mmol) is added to this solution. The mixture is stirred for 16 h at 55° C. in the dark and then the excess reagent is destroyed with methanol (224 μl, 5.52 mmol). The reaction medium is loaded onto a Sephadex® LH20 gel column (95×2 cm) eluted with a 75/20/5 v/v/v methanol/N,N-dimethylformamide/H₂O mixture, to give compound 36 (142 mg).

¹H NMR [500 MHz] (CDCl₃) δ of the anomeric protons Glc^{I} α 4.59 ppm, IdoUA^{II} α 5.31 ppm, Glc^{III} α 5.21 ppm, IdoUA^{IV} α 5.32 ppm, Glc^{V} α 5.20 ppm, IdoUA^{VI} α 5.32 ppm, Glc^{VII} α 5.17 ppm and IdoUA^{VIII} α 5.34 ppm.

### Methyl (lithium 4-O-benzyl-3-O-methyl-2-O-lithium sulphonato-α-L-idopyranosyluronate)-(1→4)-[(2-azido-2-deoxy-3-O-methyl-6-O-lithium sulphonato-α-D-glucopyranosyl)-(1→4)-(methyl lithium 3-O-methyl-2-O-lithium sulphonato-α-L-idopyranosyluronate)-(1→4)]₃-2-[(benzyloxy)carbonyl]amino-2-deoxy-3-O-methyl-6-O-lithium sulphonato-α-D-glucopyranoside (37)

A 0.5 M solution of lithium hydroxide in water (4.1 ml, 2.075 mmol) is added, at 0° C. under argon, to compound 36 (38 mg, 0.0152 mmol) dissolved in a 1:1 methanol/tetrahydrofuran solution (8.3 ml). After stirring at 0° C. for 19 h, the reaction medium is loaded onto a column of Sephadex® LH20 gel (95×2 cm) eluted with a 75/20/5 v/v/v methanol/N,N-dimethylformamide/water mixture, to give compound 37 (161 mg).

Rf=0.07, silica gel, ethyl acetate/pyridine/acetic acid/water (6/2/2/0.6/1)/(5/5/1/3) 9/1 v/v.

### Methyl (sodium 3-O-methyl-2-O-sodium sulphonato-α-L-idopyranosyluronate)-(1→4)-[(2-amino-2-deoxy-3-O-methyl-6-O-sodium sulphonato-α-D-glucopyranosyl)-(1→4)-(methyl sodium 3-O-methyl-2-O-sodium sulphonato-α-L-idopyranosyluronate)-(1→4)]₃-2-amino-2-deoxy-3-O-methyl-6-O-sodium sulphonato-α-D-glucopyranoside (38)

Ammonium formate (194 mg, 3.071 mmol) and 10% palladium-on-carbon (385 mg) are added, under an inert atmosphere, to a solution of compound 37 (154 mg, 0.061 mmol) in 1/1 v/v tert-butanol/water (12 ml). After stirring at ambient temperature for 4 h 15 min, the reaction medium is filtered (Millipore® filter LSWP 5 μm) and concentrated to dryness. The residue is loaded onto a column of Sephadex® G25-fine gel (95×2 cm) eluted with a 0.2 M aqueous solution of NaCl. The fractions containing the expected compound are combined, and loaded onto a column of Sephadex® G25-fine gel (95×2 cm) eluted with water. The product 38 is obtained (85.5 mg).

¹H NMR [500 MHz] (D₂O) δ of the anomeric protons Glc^{I} α 4.97 ppm, IdoUA^{II} α 5.32 ppm, Glc^{III} α 5.14 ppm, IdoUA^{IV} α 5.32 ppm, Glc^{V} α 5.14 ppm, IdoUA^{VI} α 5.32 ppm, Glc^{VII} α 5.14 ppm and IdoUA^{VIII} α 5.21 ppm.

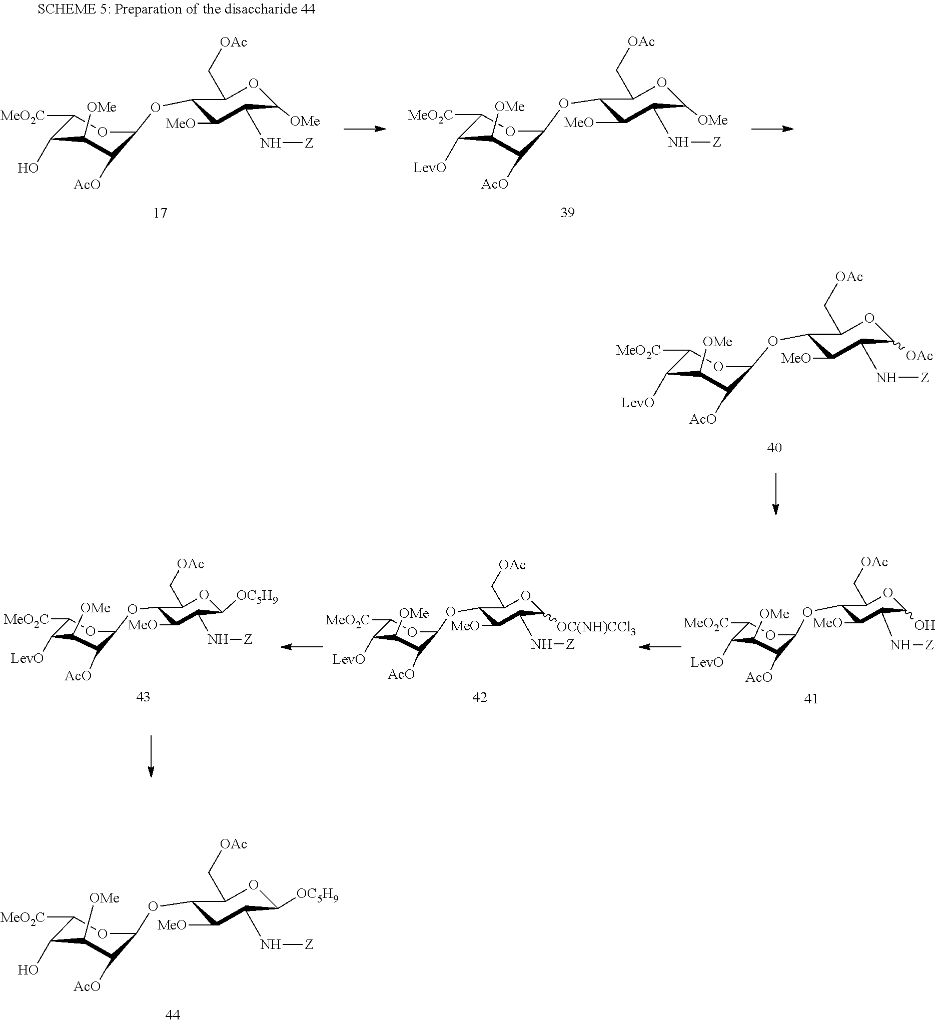

### Methyl (methyl 2-O-acetyl-4-O-levulinoyl-3-O-methyl-α-L-idopyranosyluronate)-(1→4)-6-O-acetyl-2-[(benzyloxy)carbonyl]amino-2-deoxy-3-O-methyl-α-D-glucopyranoside (39)

4-dimethylaminopyridine (76 mg, 0.626 mmol), 1-(3-dimethylaminopropyl)-3-ethylcarbodiimide hydrochloride (1.2 g, 6.26 mmol) and levulinic acid (643 μl, 6.26 mmol) are added successively, under an inert atmosphere, to a solution of compound 17 in dioxane (63 ml). After stirring at ambient temperature for 5 h 45 min, 4-dimethylaminopyridine (38 mg, 0.313 mmol), 1-(3-dimethylaminopropyl)-3-ethylcarbodiimide hydrochloride (0.6 g, 3.13 mmol) and levulinic acid (322 μl, 3.13 mmol) are again successively added. After stirring at ambient temperature for 16 h, the reaction medium is concentrated and the residue is placed in solution in dichloromethane. The organic phase is washed successively with a 10% aqueous solution of potassium hydrogen sulphate, with a 2% aqueous solution of sodium hydrogen carbonate and then with a saturated solution of sodium chloride. The organic phase is then dried over sodium sulphate, filtered and then evaporated to dryness. The residue is purified by flash chromatography on a silica gel column (cyclohexane/acetone), to give 2.23 g of compound 39.

¹H NMR [500 MHz] (CDCl₃) δ of the anomeric protons Glc^{I} 4.96 ppm and IdoUA^{II} 5.07 ppm.

### (Methyl 2-O-acetyl-4-O-levulinoyl-3-O-methyl-α-L-idopyranosyluronate)-(1→4)-1,6-di-O-acetyl-2-[(benzyloxy)carbonyl]amino-2-deoxy-3-O-methyl-α,β-D-glucopyranose (40)

96% sulphuric acid (179 μl) previously diluted in a 1/1 acetic acid/acetic anhydride solution (1.8 ml) is added, at 0° C. under an argon atmosphere, to a solution of compound 39 (1.29 g, 1.79 mmol) in a 1/1 acetic acid/acetic anhydride solution (27 ml). After stirring at ambient temperature for 3 h 30 min, the progression of the reaction is stopped by adding triethylamine (25 ml). The reaction medium is coevaporated with toluene. The residue obtained is purified by flash chromatography on a silica gel column (dichloromethane/acetone), to give 1.59 g of compound 40.

40α LC-MS m/z 736.2 [(M+Na)⁺]. T_{R}=8.123 min

40β LC-MS m/z 736.2 [(M+Na)⁺]. T_{R}=8.043 min

### (Methyl 2-O-acetyl-4-O-levulinoyl-3-O-methyl-α-L-idopyranosyluronate)-(1→4)-6-O-acetyl-2-[(benzyloxy)carbonyl]amino-2-deoxy-3-O-methyl-α,β-D-glucopyranose (41)

Benzylamine (2.7 ml, 25.1 mmol) and acetic acid (38 μl, 0.662 mmol) are added successively, under an argon atmosphere, to a solution of compound 40 (500 mg, 0.662 mmol) in tetrahydrofuran (26.5 ml). After magnetic stirring for 9 h, the reaction medium is neutralized with Dowex AG 50 WX4 H⁺ resin, filtered and then concentrated. The residue is purified by flash chromatography on a silica gel column (toluene/acetone), to give 335 mg of compound 41.

Rf=0.3, silica gel, 1/1 v/v toluene/acetone

### (Methyl 2-O-acetyl-4-O-levulinoyl-3-O-methyl-α-L-idopyranosyluronate)-(1→4)-6-O-acetyl-2-[(benzyloxy)carbonyl]amino-2-deoxy-3-O-methyl-α,β-D-glucopyranose trichloroacetimidate (42)

Trichloroacetonitrile (528 μl, 5.27 mmol) and caesium carbonate (233 mg, 1.69 mmol) are added, at ambient temperature under an argon atmosphere, to a solution of compound 41 (752.5 mg, 1.05 mmol) in dichloromethane (21 ml). After stirring at ambient temperature for 16 h, the reaction medium is filtered through Celite® and then concentrated. The residue is purified by chromatography on a silica gel column (toluene/acetone+0.1% triethylamine), to give 633 mg of compound 42.

42α ¹H NMR [500 MHz] (CDCl₃) δ of the anomeric protons Glc^{I}: 6.27 ppm and IdoUA^{II} 5.07 ppm.

42β ¹H NMR [500 MHz] (CDCl₃) δ of the anomeric protons Glc^{I}: 5.95 ppm and IdoUA^{II} 5.07 ppm.

### Pent-4-ene (methyl 2-O-acetyl-4-O-levulinoyl-3-O-methyl-α-L-idopyranosyluronate)-(1→4)-6-O-acetyl-2-[(benzyloxy)carbonyl]amino-2-deoxy-3-O-methyl-β-D-glucopyranoside (43)

A mixture of trichloroacetimidate 42 (130.9 mg, 0.153 mmol), of 4-penten-1-ol (78 μL, 0.763 mmol) and of 4 Å molecular sieve powder (130 mg) in dichloromethane (6.9 ml) is stirred under an argon atmosphere for 1 h 45 min at ambient temperature. The reaction mixture is cooled to −20° C. and a 1M solution of tert-butyldimethylsilyl triflate in dichloromethane (30.5 μL, 0.0305 mmol) is added dropwise. After stirring at −20° C. for 35 min, a further addition of solution of tert-butyldimethylsilyl triflate in dichloromethane (15 μl, 0.015 mmol) is carried out. After stirring at −20° C. for 10 min, the reaction is neutralized by adding solid sodium hydrogen carbonate. The reaction medium is filtered through Celite® and then evaporated. The residue is purified by chromatography on a silica gel column (dichloromethane/acetone), to give 379 mg of compound 43.

¹H NMR [500 MHz] (CDCl₃) δ of the anomeric protons Glc^{I}: 4.63 ppm and IdoUA^{II} 5.04 ppm.

### Pent-4-ene (methyl 2-O-acetyl-3-O-methyl-α-L-idopyranosyluronate)-(1→4)-6-O-acetyl-2-[(benzyloxy)carbonyl]amino-2-deoxy-3-O-methyl-β-D-glucopyranoside (44)

Hydrazine acetate (220 mg, 2.4 mmol) is added to a solution of compound 43 in a 1/2 toluene/ethanol mixture (96 ml). The reaction medium is stirred for 40 min at ambient temperature. After concentration, the residue is taken up in dichloromethane and then washed with water. After drying over sodium sulphate, filtration and then concentration, the residue is chromatographed on a silica gel column (dichloromethane/acetone), to give 315 mg of compound 44.

¹H NMR [500 MHz] (CDCl₃) δ of the anomeric protons Glc^{I} 4.66 ppm and IdoUA^{II} 4.99 ppm

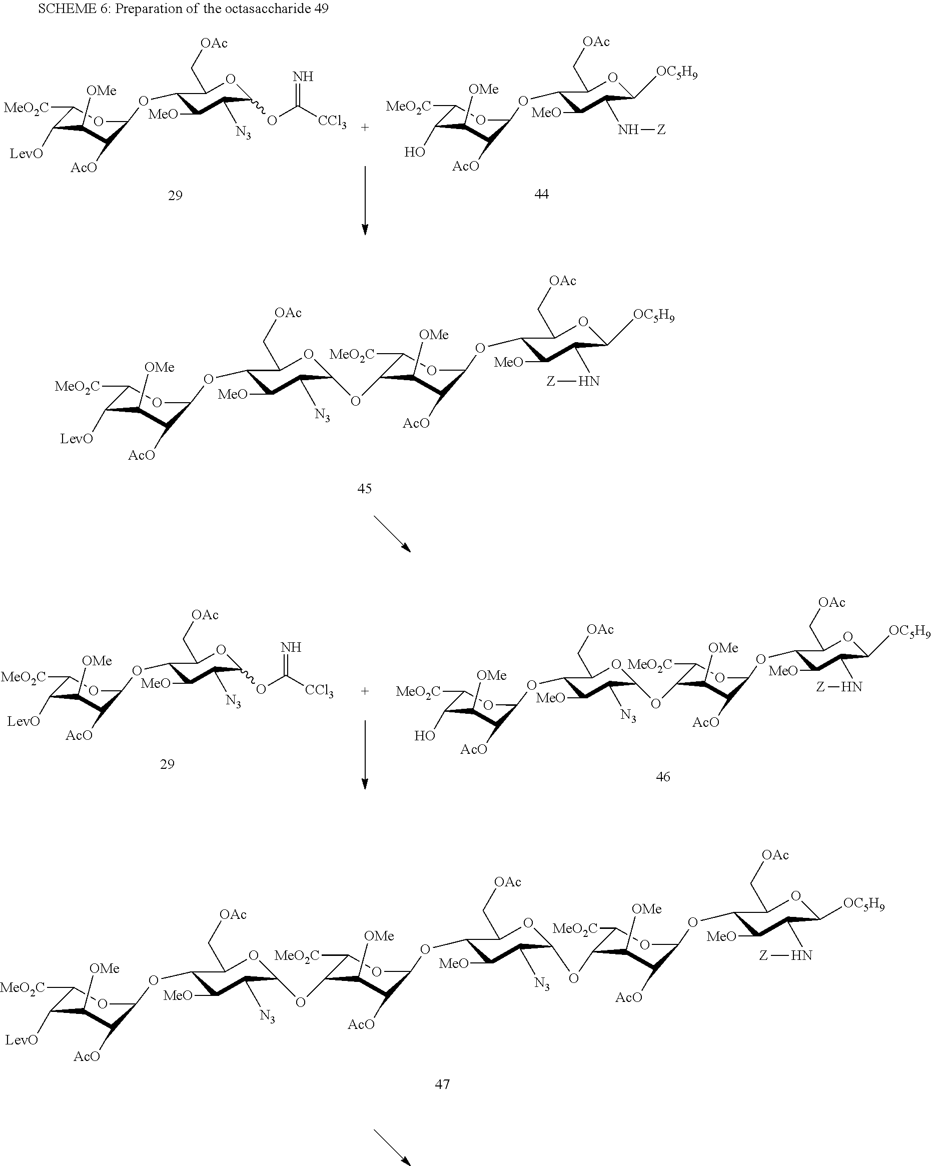

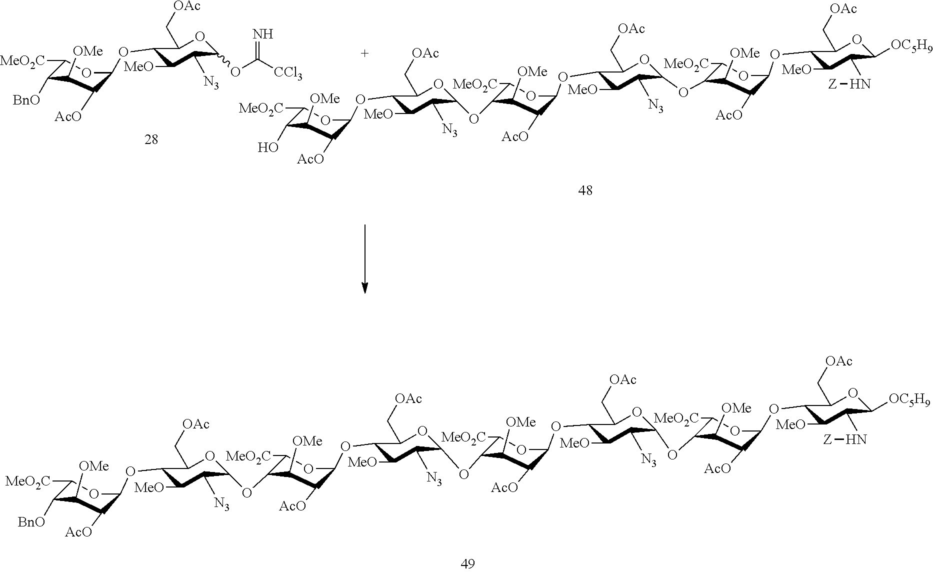

### Pent-4-ene (methyl 2-O-acetyl-3-O-methyl-4-O-levulinoyl-α-L-idopyranosyluronate)-(1→4)-(6-O-acetyl-2-azido-2-deoxy-3-O-méthyl-α-D-glucopyranosyl)-(1→4)-(methyl 2-O-acetyl-3-O-methyl-α-L-idopyranosyluronate)-(1→4)-6-O-acetyl-2-[(benzyloxy)carbonyl]amino-2-deoxy-3-O-methyl-β-D-glucopyranoside (45)

Compound 29 (427 mg, 0.57 mmol) and compound 44 (300 mg, 0.439 mmol) are processed according to the same procedure as that described for the preparation of 30, to give compound 45 (340 mg).

¹H NMR [500 MHz] (CDCl₃) δ of the anomeric protons Glc^{I}: 4.68, IdoUA^{II}: 5.06, Glc^{I}: 4.98 and IdoUA^{IV}: 5.08.

### Pent-4-ene (methyl 2-O-acetyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-6-O-acetyl-2-[(benzyloxy)carbonyl]amino-2-deoxy-3-O-methyl-β-D-glucopyranoside (46)

Compound 45 (374 mg, 0.294 mmol) is processed according to the same procedure as that described for the preparation of 31, to give compound 46 (386 mg).

¹H NMR [500 MHz] (CDCl₃) δ of the anomeric protons Glc^{I}: 4.68, IdoUA^{II}: 5.06, Glc^{III}: 4.99 and IdoUA^{IV}: 5.00.

### Pent-4-ene (methyl 2-O-acetyl-4-O-levulinoyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-6-O-acetyl-2-[(benzyloxy)carbonyl]amino-2-deoxy-3-O-methyl-β-D-glucopyranoside (47)

Compound 29 (75.5 mg, 0.101 mmol) and compound 46 (91 mg, 0.0776 mmol) are processed according to the same procedure as that described for the preparation of 32, to give compound 47 (388 mg).

¹H NMR [500 MHz] (CDCl₃) δ of the anomeric protons Glc^{I}: 4.67, IdoUA^{II}: 5.07, Glc^{III}: 4.98, IdoUA^{IV}: 5.10, Glc^{V}: 5.02 and IdoUA^{VI}: 5.08.

### Pent-4-ene (methyl 2-O-acetyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-6-O-acetyl-2-[(benzyloxy)carbonyl]amino-2-deoxy-3-O-methyl-β-D-glucopyranoside (48)

Compound 47 (382 mg, 0.217 mmol) is processed according to the same procedure as that described for the preparation of 33, to give compound 48 (327 mg).

¹H NMR [500 MHz] (CDCl₃) δ of the anomeric protons Glc^{I}: 4.67 IdoUA^{II}: 5.07, Glc^{III}: 4.98, IdoUA^{IV}: 5.09, Glc^{V}: 5.02, IdoUA^{VI}: 5.00.

### Pent-4-ene (methyl 2-O-acetyl-4-O-benzyl-3-O-methyl-α-L-idopyranosyluronate)-(1→4)-(6-O-acetyl-2-azido-2-deoxy-3-O-methyl-α-D-glucopyranosyl)-(1→4) (methyl 2-O-acetyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-6-O-acetyl-2-[(benzyloxy)carbonyl]amino-2-deoxy-3-O-methyl-β-D-glucopyranoside (49)

Compound 28 (185.9 mg, 0.251 mmol) and compound 48 (321 mg, 0.193 mmol) are processed according to the same procedure as that described for the preparation of 34, to give compound 49 (209 mg).

¹H NMR [500 MHz] (CDCl₃) δ of the anomeric protons Glc^{I}: 4.67 ppm, IdoUA^{II}: 5.07 ppm, Glc^{I}: 4.98 ppm, IdoUA^{IV}: 5.09 ppm, Glc^{V}: 5.02 ppm, IdoUA^{VI}: 5.08 ppm, Glc^{VII}: 5.00 ppm and IdoUA^{VIII}: 5.13 ppm.

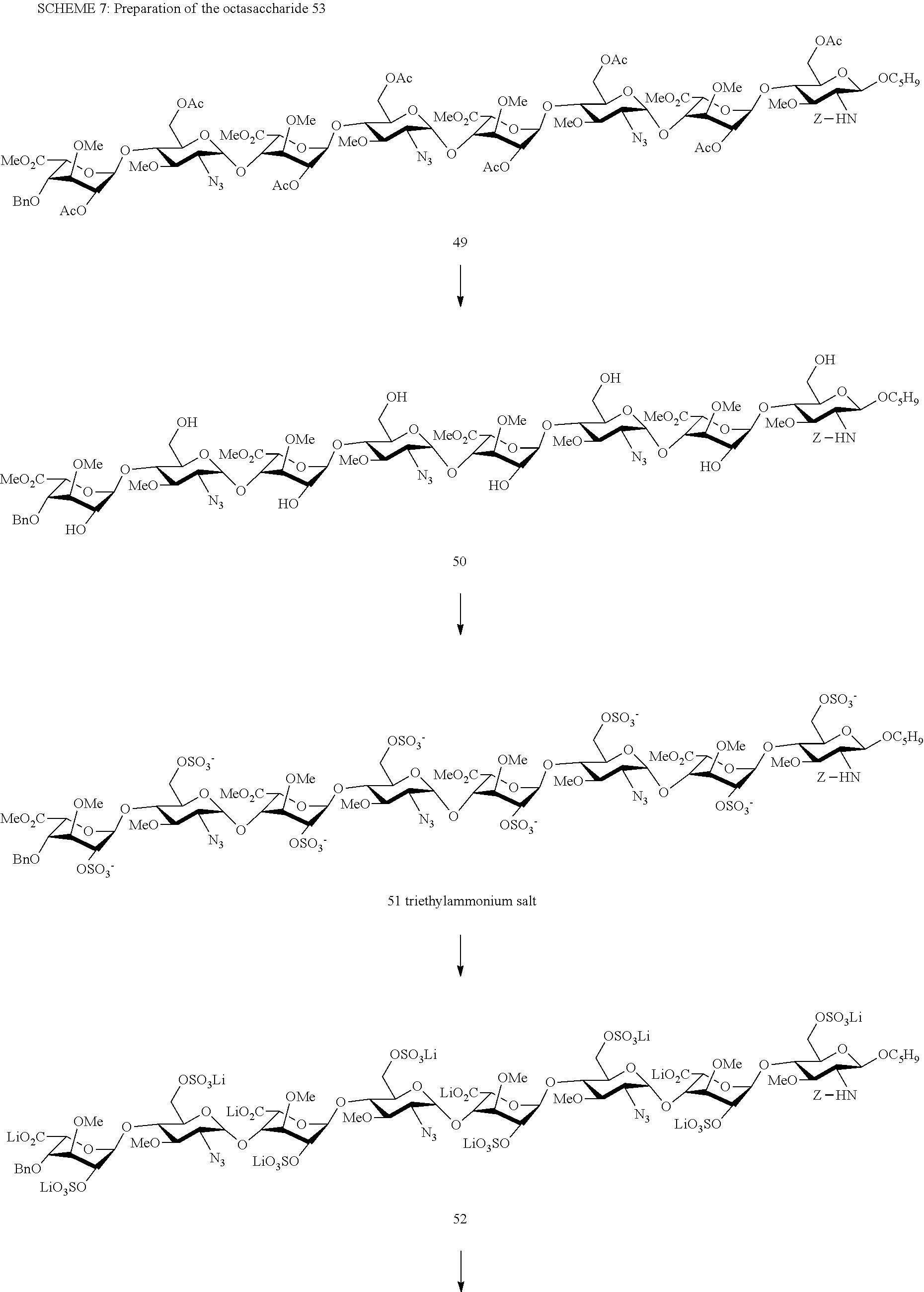

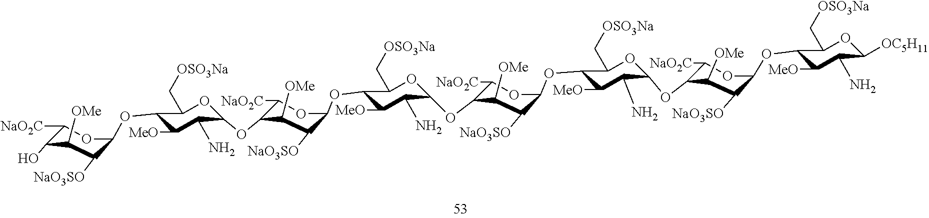

### Pent-4-ene (methyl 4-O-benzyl-3-O-methyl-α-L-idopyranosyluronate)-(1→4)-(2-azido-2-deoxy-3-O-methyl-α-D-glucopyranosyl)-(1→4)-(methyl 3-O-methyl-α-L-idopyranosyluronate)-(1→4)-(2-azido-2-deoxy-3-O-methyl-α-D-glucopyranosyl)-(1→4)-(methyl 3-O-methyl-α-L-idopyranosyluronate)-(1→4)-(2-azido-2-deoxy-3-O-methyl-α-D-glucopyranosyl)-(1→4)-(methyl 3-O-methyl-α-L-idopyranosyluronate)-(1→4)-2-[(benzyloxy)carbonyl]amino-2-deoxy-3-O-methyl-β-D-glucopyranoside (50)

A 0.5M solution of sodium methoxide in methanol (67 μl) is added, at 0° C., under an argon atmosphere, to a solution of compound 49 (50 mg, 0.0223 mmol) in a 2/3 v/v dichloromethane/methanol mixture (6.7 ml) containing 3 Å molecular sieve (29 mg). After magnetic stirring at 0° C. for 3 h, at ambient temperature for 4 h 45 min, at −18° C. for 16 h, and then at ambient temperature for 2 h, the reaction medium is neutralized with Dowex® 50WX4 H⁺ resin. After filtration and concentration under vacuum, the residue is purified by size exclusion chromatography (Sephadex® LH20, 120×3 cm, 7/2/1 methanol/N,N-dimethylformamide/water), to give 38.7 mg of compound 50.

¹H NMR [500 MHz] (CDCl₃) δ of the anomeric protons Glc^{I}: 4.69, IdoUA^{II}: 5.16, Glc^{III}: 5.05, IdoUA^{IV}: 5.18, Glc^{V}: 5.06, IdoUA^{VI}: 5.18, Glc^{VII}: 5.04 and IdoUA^{VIII}: 5.19.

### Pent-4-ene (methyl 4-O-benzyl-3-O-methyl-2-O-triethylammonium sulphonato-α-L-idopyranosyluronate)-(1→4)-(2-azido-2-desoxy-3-O-methyl-6-O-triethylammonium sulphonato-α-D-glucopyranosyl)-(1→4)-(methyl 3-O-methyl-2-O-triethylammonium sulphonato-α-L-idopyranosyluronate)-(1→4)-(2-azido-2-desoxy-3-O-methyl-6-O-triethylammonium sulphonato-α-D-glucopyranosyl)-(1→4)-(methyl 3-O-methyl-2-O-triethylammonium sulphonato-α-L-idopyranosyluronate)-(1→4)-(2-azido-2-desoxy-3-O-methyl-6-O-triethylammonium sulphonato-α-D-glucopyranosyl)-(1→4)-(methyl 3-O-methyl-2-O-triethylammonium sulphonato-α-L-idopyranosyluronate)-(1→4)-2-[(benzyloxy)carbonyl]amino-2-désoxy-3-O-methyl-6-O-triethylammonium sulphonato-6-D-glucopyranoside (51)

Compound 50 (37.5 mg, 0.0197 mmol) is processed according to the same procedure as that described for the preparation of 36, to give compound 51 (56.1 mg).

¹H NMR [500 MHz] (CDCl₃) δ of the anomeric protons Glc^{I}: 4.39, IdoUA^{II}: 5.23, Glc^{III}: 5.15, IdoUA^{IV}: 5.30, Glc^{V}: 5.15, IdoUA^{VI}: 5.30, Glc^{VII}: 5.13 and IdoUA^{VIII}: 5.32.

### Pent-4-ene (lithium 4-O-benzyl-3-O-methyl-2-O-lithium sulphonato-α-L-idopyranosyluronate)-(1→4)-(2-azido-2-deoxy-3-O-methyl-6-O-lithium sulphonato-α-D-glucopyranosyl)-(1→4)-(lithium 3-O-methyl-2-O-lithium sulphonato-α-L-idopyranosyluronate)-(1→4)-(2-azido-2-deoxy-3-O-methyl-6-O-lithium sulphonato-α-D-glucopyranosyl)-(1→4)-(lithium 3-O-methyl-2-O-lithium sulphonato-α-L-idopyranosyluronate)-(1→4)-(2-azido-2-deoxy-3-O-methyl-6-O-lithium sulphonato-α-D-glucopyranosyl)-(1→4)-(lithium 3-O-methyl-2-O-lithium sulphonato-α-L-idopyranosyluronate)-(1→4)-2-[(benzyloxy)carbonyl]amino-2-deoxy-3-O-methyl-6-O-lithium sulphonato-β-D-glucopyranoside (52)

Compound 51 (71.3 mg, 0.0212 mmol) is processed according to the same procedure as that described for the preparation of 37, to give compound 52 (58.7 mg).

Rf=0.29, silica gel, ethyl acetate/pyridine/acetic acid/water (6/2/2/0.6/1)/(5/5/1/3) 1/8 v/v.

### Pentyl (sodium 3-O-methyl-2-O-sodium sulphonato-α-L-idopyranosyluronate)-(1→4)-(2-amino-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-amino-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-amino-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-2-amino-2-deoxy-3-O-methyl-6-O-sodium sulphonato-β-D-glucopyranoside (53)

Ammonium formate (27 mg, 0.426 mmol) and 10% palladium-on-carbon (54.5 mg) are added, under an inert atmosphere, to a solution of compound 52 (21.8 mg, 0.0085 mmol) in a 1/1 v/v tert-butanol/water mixture (1.7 ml). After stirring at ambient temperature for 3 h 30 min, the reaction medium is filtered (Millipore® LSWP 5 μm filter) and concentrated to dryness. The residue is loaded onto a column of Sephadex® G25-fine gel (95×2 cm) eluted with a 0.2 M aqueous solution of NaCl. The fractions containing the expected compound are combined, and loaded onto a column of Sephadex® G25-fine gel (95×2 cm) eluted with water. The resulting crude product 53 (17.3 mg) is used as it is in the next step.

¹H NMR [500 MHz] (CDCl₃) δ of the anomeric protons Glc^{I}: 4.75, IdoUA^{II}: 5.24, Glc^{III}: 5.43, IdoUA^{IV}: 5.26, Glc^{V}α: 5.43 IdoUA^{VI}: 5.26, Glc^{VII}: 5.43 and IdoUA^{VIII}: 5.18.

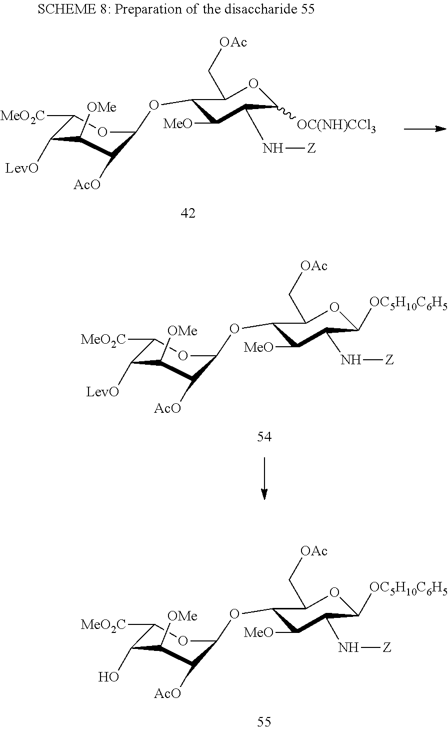

### 5-Phenylpentyl (methyl 2-O-acetyl-4-O-levulinoyl-3-O-methyl-α-L-idopyranosyluronate)-(1→4)-6-O-acetyl-2-[(benzyloxy)carbonyl]amino-2-deoxy-3-O-methyl-β-D-glucopyranoside (54)

Compound 42 (519.8 mg, 0.606 mmol) is processed according to the same procedure as that described for the preparation of 43, to give compound 54 (483.1 mg).

¹H NMR [500 MHz] (CDCl₃) δ of the anomeric protons Glc^{I}: 4.61 ppm and IdoUA^{II}: 5.03 ppm.

### 5-Phenylpentyl (methyl 2-O-acetyl-3-O-methyl-α-L-idopyranosyluronate)-(1→4)-6-O-acetyl-2-[(benzyloxy)carbonyl]amino-2-deoxy-3-O-methyl-β-D-glucopyranoside (55)

Compound 54 (396.4 mg, 0.461 mmol) is processed according to the same procedure as that described for the preparation of 44, to give compound 55 (383.4 mg).

¹H NMR [500 MHz] (CDCl₃) δ of the anomeric protons Glc^{I}: 4.69 ppm and IdoUA^{II}: 5.0 ppm.

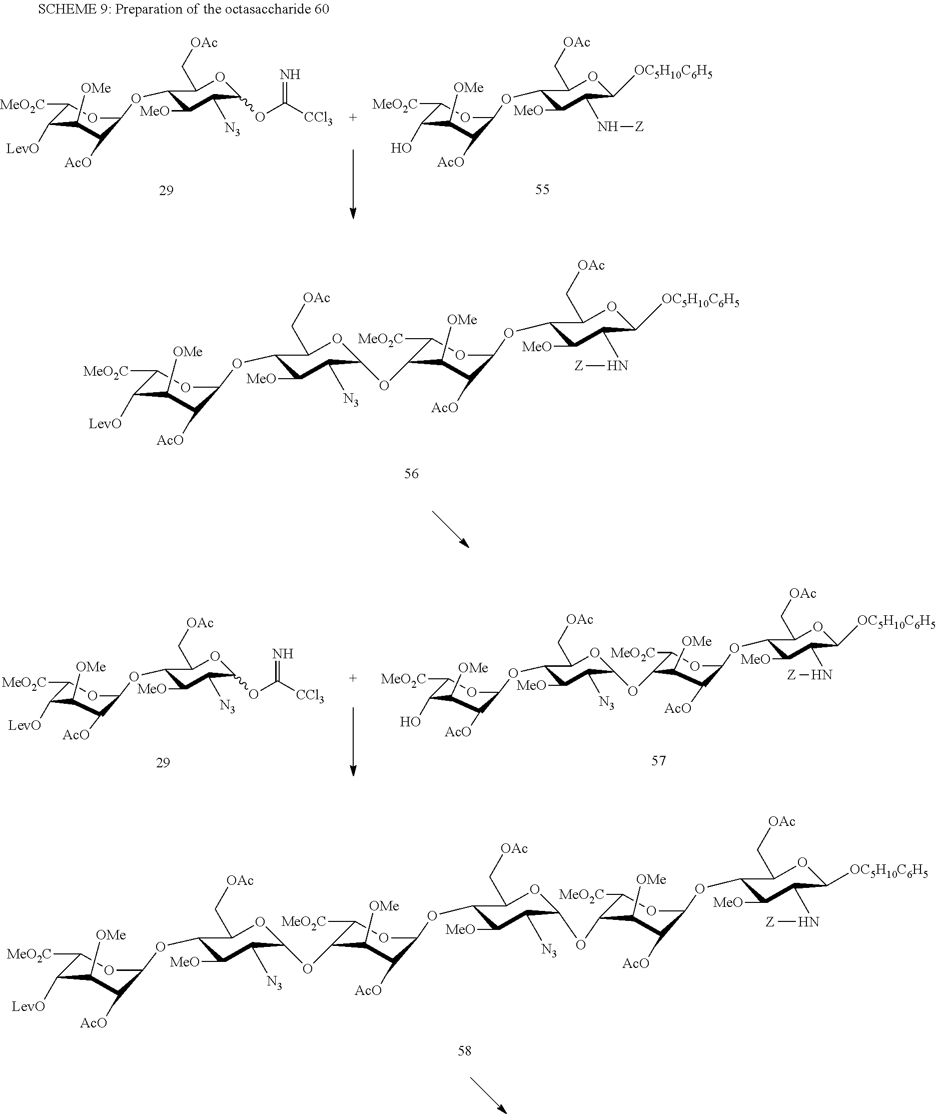

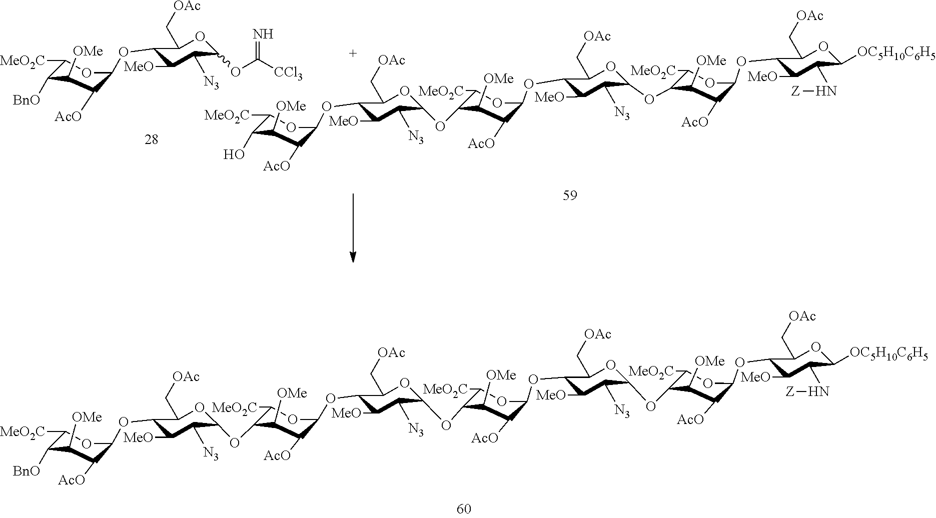

### 5-Phenylpentyl (methyl 2-O-acetyl-3-O-methyl-4-O-levulinoyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-6-O-acetyl-2-[(benzyloxy)carbonyl]amino-2-deoxy-3-O-methyl-β-D-glucopyranoside (56)

Compound 29 (1.17 g, 1.22 mmol) and compound 55 (1.50 g, 1.59 mmol) are processed according to the same procedure as that described for the preparation of 30, to give compound 56 (1.79 g).

¹H NMR [500 MHz] (CDCl₃) δ of the anomeric protons Glc^{I} β: 4.5, IdoUA^{II}: 5.00.

### 5-Phenylpentyl (methyl 2-O-acetyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-6-O-acetyl-2-[(benzyloxy)carbonyl]amino-2-deoxy-3-O-methyl-β-D-glucopyranoside (57)

Compound 56 (495 mg, 0.367 mmol) is processed according to the same procedure as that described for the preparation of 31, to give compound 57 (442 mg).

¹H NMR [500 MHz] (CDCl₃) δ of the anomeric protons Glc^{I} β: 4.65 and IdoUA^{II}: 5.07 and Glc^{III} α: 4.99 and IdoUA^{IV}: 5.01.

### 5-Phenylpentyl (methyl 2-O-acetyl-4-O-levulinoyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-6-O-acetyl-2-[(benzyloxy)carbonyl]amino-2-deoxy-3-O-methyl-β-D-glucopyranoside (58)

Compound 57 (433.7 mg, 0.347 mmol) and compound 29 (338 mg, 0.45 mmol) are processed according to the same procedure as that described for the preparation of 32, to give compound 58 (534 mg).

LC-MS m/z 1860 [(M+Na)⁺]. T_{R}=17.02 min

### 5-Phenylpentyl (methyl 2-O-acetyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-6-O-acetyl-2-[(benzyloxy)carbonyl]amino-2-deoxy-3-O-methyl-β-D-glucopyranoside (59)

Compound 58 (447 mg, 0.243 mmol) is processed according to the same procedure as that described for the preparation of 33, to give compound 59 (386 mg).

LC-MS m/z 1762 [(M+Na)⁺] T_{R1}=18.32 min

### 5-Phenylpentyl (methyl 2-O-acetyl-4-O-benzyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-(6-O-acetyl-2-azido-2-deoxy-3-O-methyl-α-D-glucopyranosyl)-(1→4)-(methyl 2-O-acetyl-3-O-methyl-α-L-idopyranosyluronate)-(1→4)-6-O-acetyl-2-[(benzyloxy)carbonyl]amino-2-deoxy-3-O-methyl-β-D-glucopyranoside (60)

Compound 59 (95.2 mg, 0.0547 mmol) and compound 28 (52.7 mg, 0.071 mmol) are processed according to the same procedure as that described for the preparation of 34, to give compound 60 (389 mg).

LC-MS m/z 1180.5 [(M+2H+CH₃CN)²⁺]T_{R1}=18.27 min

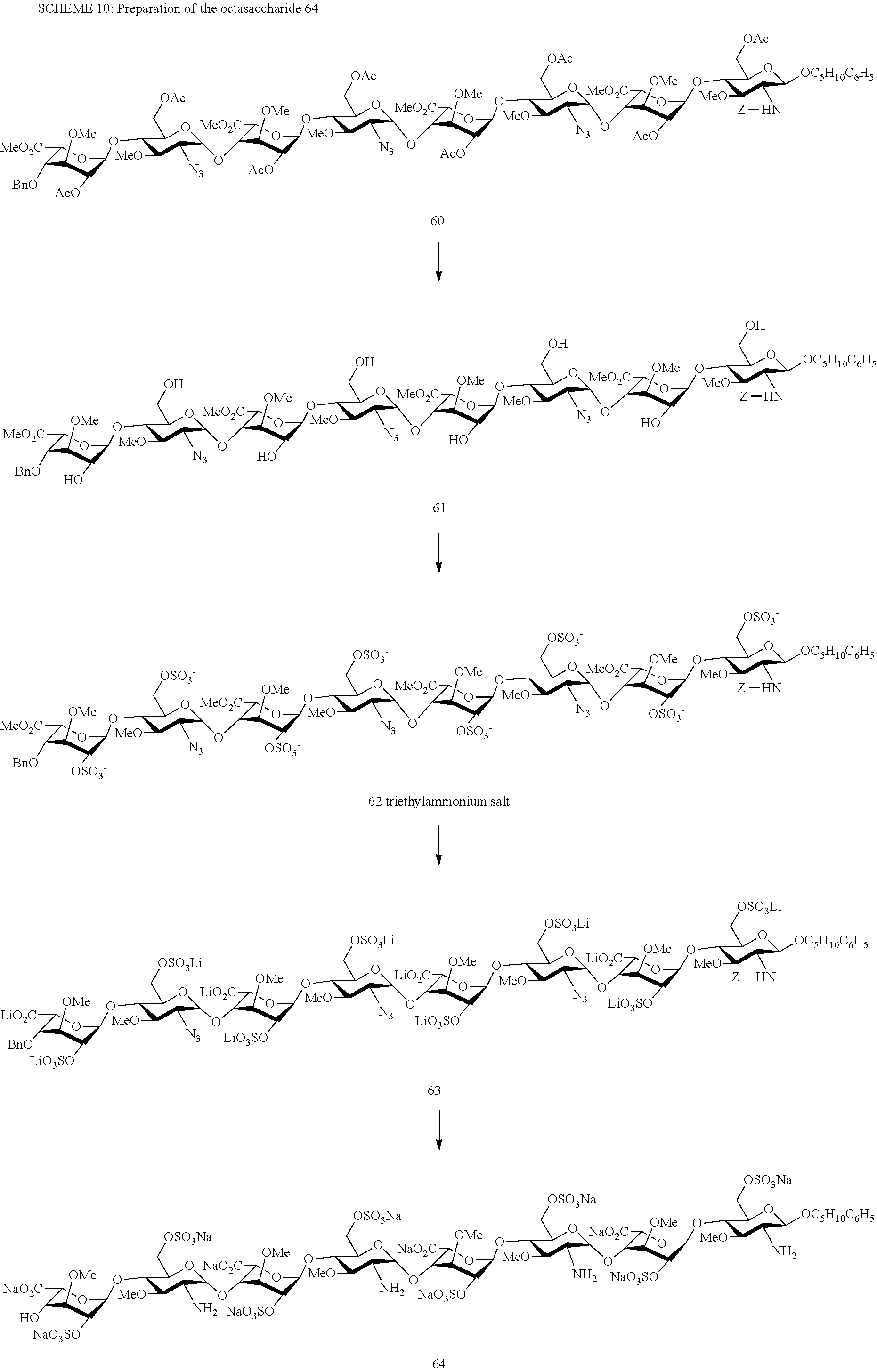

### 5-Phenylpentyl (methyl 3-O-methyl-4-O-benzyl-α-L-idopyranosyluronate)-(1→4)-(2-azido-2-deoxy-3-O-methyl-α-D-glucopyranosyl)-(1→4)-(methyl 3-O-methyl-α-L-idopyranosyluronate)-(1→4)-(2-azido-2-deoxy-3-O-methyl-α-D-glucopyranosyl)-(1→4)-(methyl 3-O-methyl-α-L-idopyranosyluronate)-(1→4)-(2-azido-2-deoxy-3-O-methyl-α-D-glucopyranosyl)-(1→4)-(methyl 3-O-methyl-α-L-idopyranosyluronate)-(1→4)-2-[(benzyloxy)carbonyl]amino-2-deoxy-3-O-methyl-β-D-glucopyranoside (61)

A 0.5 M solution of sodium methoxide in methanol (129 μl) is added, at 0° C., under an argon atmosphere, to a solution of compound 60 (100 mg, 0.0431 mmol) in a 1/1 v/v dichloromethane/methanol mixture (15.6 ml) containing 3 Å sieve (54 mg). After magnetic stirring at 0° C. for 4 h 50 min, at ambient temperature for 3 h 50 min and at −18° C. for 15 h, the reaction medium is neutralized with Dowex® 50WX4 H⁺ resin. After filtration and concentration under vacuum, the residue is purified by size exclusion chromatography (Sephadex® LH20, 120×3 cm, 75/20/5 methanol/N,N-dimethylformamide/water), to give 88.1 mg of compound 61.

¹H NMR [500 MHz] (CDCl₃) δ of the anomeric protons Glc^{I} β: 4.52, IdoUA^{II}: 5.18, Glc^{III} α: 5.05, IdoUA^{IV}: 5.18, Glc^{II} α: 5.05, IdoUA^{VI}: 5.18, Glc^{VII} α: 5.05 and IdoUA^{VIII}: 5.14.

### 5-Phenylpentyl (methyl 3-O-methyl-4-O-benzyl-2-O-triethylammonium sulphonato-α-L-idopyranosyluronate)-(1→4)-(2-azido-2-deoxy-3-O-methyl-6-O-triethylammonium sulphonato-α-D-glucopyranosyl)-(1→4)-(methyl 3-O-methyl-2-O-triethylammonium sulphonato-α-L-idopyranosyluronate)-(1→4)-(2-azido-2-deoxy-3-O-methyl-6-O-triethylammonium sulphonato-α-D-glucopyranosyl)-(1→4)-(methyl 3-O-methyl-2-O-triethylammonium sulphonato-α-L-idopyranosyluronate)-(1→4)-(2-azido-2-deoxy-3-O-methyl-6-O-triethylammonium sulphonato-α-D-glucopyranosyl)-(1→4)-(methyl 3-O-methyl-2-O-triethylammonium sulphonato-α-L-idopyranosyluronate)-(1→4)-2-[(benzyloxy)carbonyl]amino-2-deoxy-3-O-methyl-6-O-triethylammonium sulphonato-6-D-glucopyranoside (62)

Compound 61 (175 mg, 0.0882 mmol) is processed according to the same procedure as that described for the preparation of 36, to give compound 62 (229 mg).

¹H NMR [500 MHz] (CDCl₃) δ of the anomeric protons Glc^{I} β: 4.39, IdoUA^{II}: 5.32, Glc^{III} α: 5.20, IdoUA^{IV}: 5.32, Glc^{V} α: 5.20, IdoUA^{VI}: 5.32, Glc^{VII} α: 5.20 and IdoUA^{VIII}: 5.32.

### 5-Phenylpentyl (lithium 3-O-methyl-4-O-benzyl-2-O-lithium sulphonato-α-L-idopyranosyluronate)-(1→4)-(2-azido-2-deoxy-3-O-methyl-6-O-lithium sulphonato-α-D-glucopyranosyl)-(1→4)-(lithium 3-O-methyl-2-O-lithium sulphonato-α-L-idopyranosyluronate)-(1→4)-(2-azido-2-deoxy-3-O-methyl-6-O-lithium sulphonato-α-D-glucopyranosyl)-(1→4)-(lithium 3-O-methyl-2-O-lithium sulphonato-α-L-idopyranosyluronate)-(1→4)-(2-azido-2-deoxy-3-O-methyl-6-O-lithium sulphonato-α-D-glucopyranosyl)-(1→4)-(lithium 3-O-methyl-2-O-lithium sulphonato-α-L-idopyranosyluronate)-(1→4)-2-[(benzyloxy)carbonyl]amino-2-deoxy-3-O-methyl-6-O-lithium sulphonato-β-D-glucopyranoside (63)

Compound 62 (52.1 mg, 0.019 mmol) is processed according to the same procedure as that described for the preparation of 37, to give compound 63 (44.3 mg).

¹H NMR [500 MHz] (CDCl₃) δ of the anomeric protons Glc^{I} β: 4.49, IdoUA^{II}: 5.16, Glc^{III} α: 5.30, IdoUA^{IV}: 5.18, Glc^{V} α: 5.31, IdoUA^{VI}: 5.18, Glc^{VII} α: 5.29, and IdoUA^{VIII}: 5.14.

### 5-Phenylpentyl (sodium 3-O-methyl-2-O-sodium sulphonato-α-L-idopyranosyluronate)-(1→4)-(2-amino-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-amino-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-amino-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-2-amino-2-deoxy-3-O-methyl-6-O-sodium sulphonato-β-D-glucopyranoside (64)

Compound 63 (43.1 mg, 0.0152 mmol) is processed according to the same procedure as that described for the preparation of 38, to give compound 64 (31.6 mg).

ESI-MS m/z 565.07[(M 4H)⁴⁻].

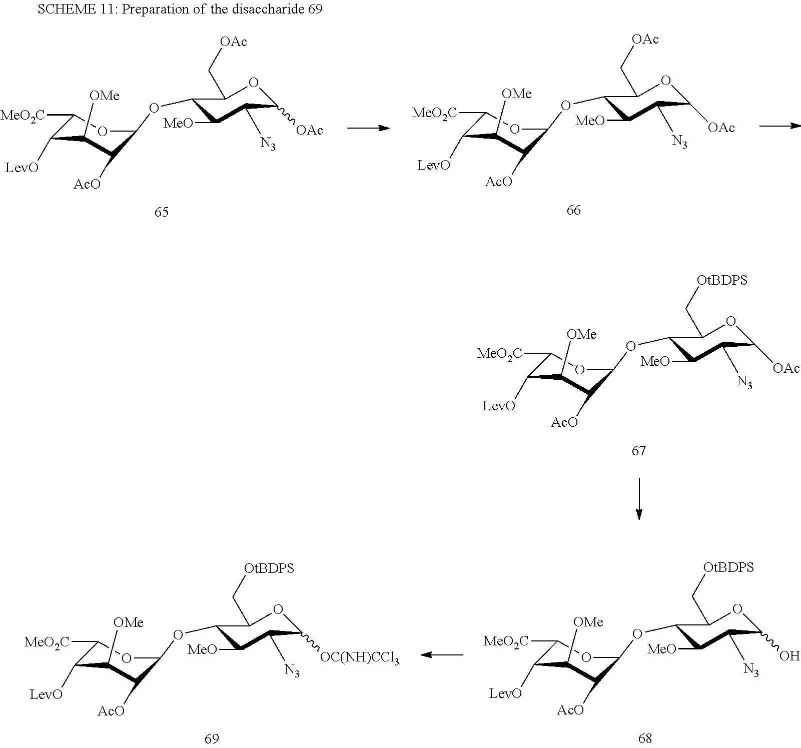

### (Methyl 2-O-acetyl-4-O-levulinoyl-3-O-methyl-α-L-idopyranosyluronate)-(1→4)-1-O-acetyl-2-azido-2-deoxy-3-O-methyl-α-D-glucopyranose (66)

[tert-Bu₂SnCl(OH)]₂ (451 mg, 1.58 mmol), prepared according to A. Orita et al., Chem. Eur. J. (2001) 7, 3321, is added, at ambient temperature under an inert atmosphere, to a solution of compound 65 (7.31 g, 11.28 mmol; WO2010/029185) in 1/1 methanol/tetrahydrofuran (144 ml). After magnetic stirring at 35° C. for 5 h, the reaction medium is concentrated and then the residue is purified by chromatography on a silica gel column (cyclohexane/acetone), to give 4.33 g of compound 66.

¹H NMR [500 MHz] (CDCl₃) δ of the anomeric protons IdoUA^{II}: 5.18 Glc^{I} α:6.18.

### (Methyl 2-O-acetyl-4-O-levulinoyl-3-O-methyl-α-L-idopyranosyluronate)-(1→4)-1-O-acetyl-2-azido-2-deoxy-3-O-methyl-6-O-tert-butyldiphenylsilyl-α-D-glucopyranose (67)

Imidazole (109 mg, 1.60 mmol) and tert-butyldiphenylsilyl chloride (22 μl, 0.08 mmol) are added, at ambient temperature, to a solution of compound 66 (47.1 mg, 0.077 mmol) in N,N-dimethylformamide (1 ml). After magnetic stirring at 35° C. for 5 h and at ambient temperature for 17 h, the progression of the reaction is stopped by adding methanol, and the reaction medium is diluted with dichloromethane and then successively washed with a 2% potassium hydrogen sulphate solution and a saturated solution of sodium chloride. The organic phase is dried over sodium sulphate, filtered and then concentrated under vacuum. The resulting residue is purified by flash chromatography on a silica gel column (toluene/acetone), to give 62.5 mg of compound 67.

¹H NMR [500 MHz] (CDCl₃) δ of the anomeric protons IdoUA^{II}: 5.25, Glc^{I} α: 6.16.

### (Methyl 2-O-acetyl-4-O-levulinoyl-3-O-methyl-α-L-idopyranosyluronate)-(1→4)-2-azido-2-deoxy-3-O-methyl-6-O-tert-butyldiphenylsilyl-α,β-D-glucopyranose (68)

Benzylamine (29.7 ml, 272 mmol) is added, under an argon atmosphere at ambient temperature, to a solution of compound 67 (6.03 g, 7.14 mmol) in tetrahydrofuran (293 ml). After magnetic stirring for 14 h, the progression of the reaction is stopped at 0° C. by adding a 1M aqueous solution of hydrochloric acid. The organic phase is washed with water, dried over sodium sulphate, filtered and then concentrated under vacuum. The residue is purified by flash chromatography on a silica gel column (toluene/acetone), to give 3.94 g of compound 68.

¹H NMR [500 MHz] (CDCl₃) δ of the anomeric protons Glc^{I} β: 4.40, IdoUA^{II}: 5.18, Glc^{I} α: 5.19 and IdoUA^{II}: 5.25.

### (Methyl 2-O-acetyl-4-O-levulinoyl-3-O-methyl-α-L-idopyranosyluronate)-(1→4)-2-azido-2-deoxy-3-O-methyl-6-O-tert-butyldiphenylsilyl-α,β-D-glucopyranose trichloroacetimidate (69)

Compound 68 (4.34 g, 5.41 mmol) is processed according to the same procedure as that described for the preparation of 28, to give compound 69 (4.36 g).

¹H NMR [500 MHz] (CDCl₃) δ of the anomeric protons Glc^{I} β: 5.60, Glc^{I} α: 6.37 and IdoUA^{II}: 5.23.

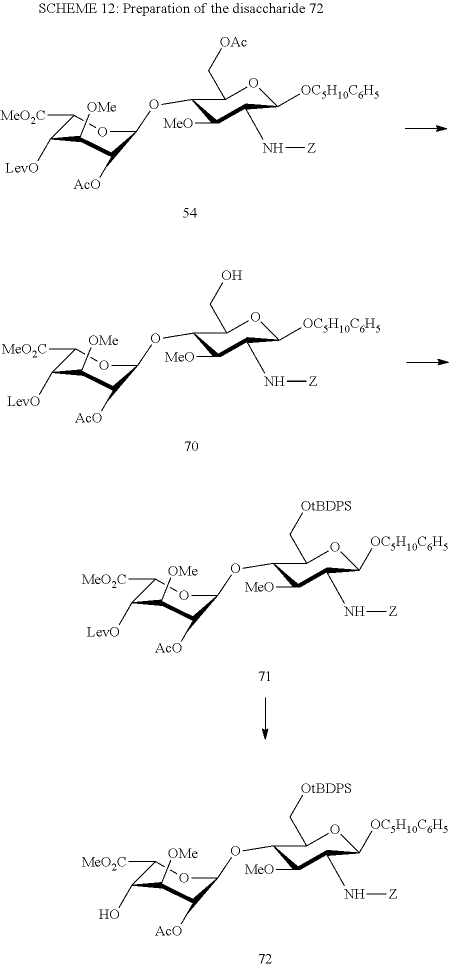

### 5-Phenylpentyl (methyl 2-O-acetyl-4-O-levulinoyl-3-O-methyl-α-L-idopyranosyluronate)-(1→4)-2-[(benzyloxy)carbonyl]amino-2-deoxy-3-O-methyl-β-D-glucopyranoside (70)

Compound 54 (1.01 g, 1.18 mmol) is processed according to the same procedure as that described for the preparation of 66, to give compound 70 (917 mg).

LC-MS m/z 840.2 [(M+Na)⁺]. T_{R}=9.478 min.

### 5-Phenylpentyl (methyl 2-O-acetyl-4-O-levulinoyl-3-O-methyl-α-L-idopyranosyluronate)-(1→4)-2-[(benzyloxy)carbonyl]amino-2-deoxy-3-O-methyl-6-O-tert-butyldiphenylsilyl-3-D-glucopyranoside (71)

Compound 70 (941 mg, 1.15 mmol) is processed according to the same procedure as that described for the preparation of 67, to give compound 71 (1.45 g).

LC-MS m/z 1078.2 [(M+Na)⁺]. T_{R}=11.585 min.

### 5-Phenylpentyl (methyl 2-O-acetyl-3-O-methyl-α-L-idopyranosyluronate)-(1→4)-2-[(benzyloxy)carbonyl]amino-2-deoxy-3-O-methyl-6-O-tert-butyldiphenylsilyl-3-D-glucopyranoside (72)

Compound 71 (1.45 g, 1.37 mmol) is processed according to the same procedure as that described for the preparation of 44, to give compound 72 (1.18 g).

LC-MS m/z 980.2 [(M+Na)⁺]. T_{R}=11.571 min.

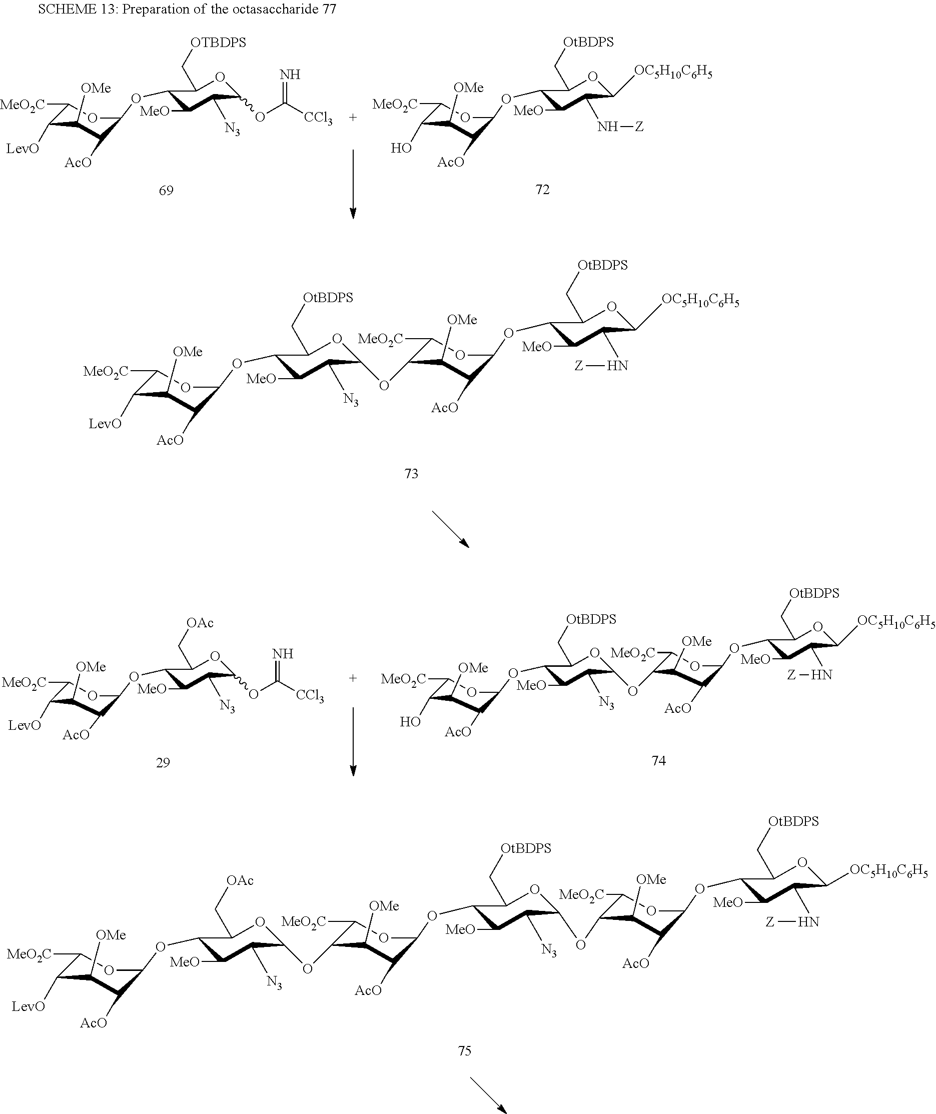

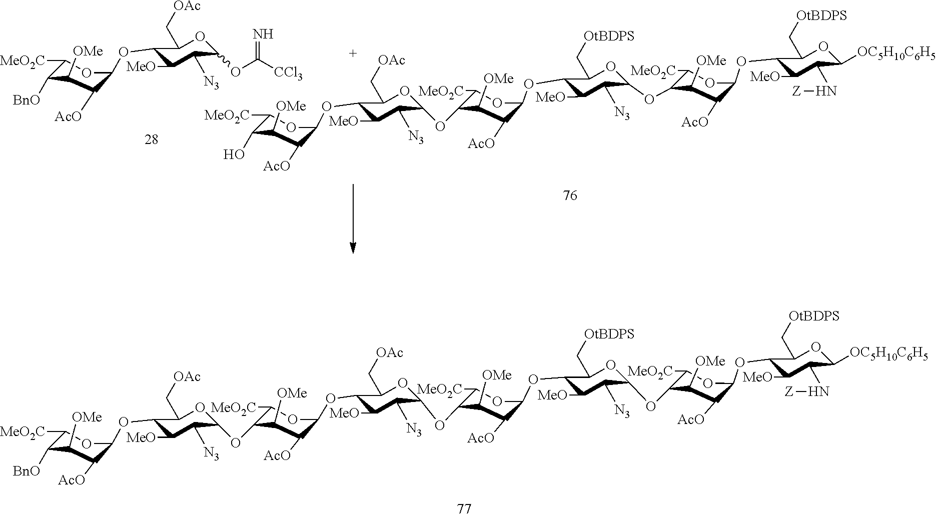

### 5-Phenylpentyl (methyl 2-O-acetyl-3-O-methyl-4-O-levulinoyl-α-L-idopyranosyluronate)-(1→4)-(2-azido-2-deoxy-3-O-methyl-6-O-tert-butyldiphenylsilyl-α-D-glucopyranosyl)-(1→4)-(methyl 2-O-acetyl-3-O-methyl-α-L-idopyranosyluronate)-(1→4)-2-[(benzyloxy)carbonyl]amino-2-deoxy-3-O-methyl-6-O-tert-butyldiphenylsilyl-β-D-glucopyranoside (73)

Compound 72 (1.17 g, 1.22 mmol) and compound 69 (1.50 g, 1.59 mmol) are processed according to the same procedure as that described for the preparation of 30, to give compound 73 (1.79 g).

¹H NMR [500 MHz] (CDCl₃) δ of the anomeric protons Glc^{I} β: 4.54 IdoUA^{II}: 5.17, Glc^{III} α: 4.94 and IdoUA^{IV}: 5.23.

### 5-Phenylpentyl (methyl 2-O-acetyl-3-O-methyl-α-L-idopyranosyluronate)-(1→4)-(2-azido-2-deoxy-3-O-methyl-6-O-tert-butyldiphenylsilyl-α-D-glucopyranosyl)-(1→4)-(methyl 2-O-acetyl-3-O-methyl-α-L-idopyranosyluronate)-(1→4)-2-[(benzyloxy)carbonyl]amino-2-deoxy-3-O-methyl-6-O-tert-butyldiphenylsilyl-β-D-glucopyranoside (74)

Compound 73 (1.78 g, 1.02 mmol) is processed according to the same procedure as that described for the preparation of 31, to give compound 74 (1.66 g).

¹H NMR [500 MHz] (CDCl₃) δ of the anomeric protons Glc^{I} β: 4.54, IdoUA^{II}: 5.17, Glc^{III} α: 4.94 and IdoUA^{IV}: 5.23.

### 5-Phenylpentyl (methyl 2-O-acetyl-3-O-methyl-4-O-levulinoyl-α-L-idopyranosyluronate)-(1→4)-(2-azido-2-deoxy-3-O-methyl-6-O-acetyl-α-D-glucopyranosyl)-(1→4)-(methyl 2-O-acetyl-3-O-methyl-α-L-idopyranosyluronate)-(1→4)-(2-azido-2-deoxy-3-O-methyl-6-O-tert-butyldiphenylsilyl-α-D-glucopyranosyl)-(1→4)-(methyl 2-O-acetyl-3-O-methyl-α-L-idopyranosyluronate)-(1→4)-2-[(benzyloxy)carbonyl]amino-2-deoxy-3-O-methyl-6-O-tert-butyldiphenylsilyl-β-D-glucopyranoside (75)

Compound 74 (1.65 g, 1.02 mmol) and compound 29 (980 mg, 1.31 mmol) are processed according to the same procedure as that described for the preparation of 32, to give compound 75 (1.75 g).

¹H NMR [500 MHz] (CDCl₃) δ of the anomeric protons Glc^{I} β: 4.54, IdoUA^{II}: 5.17, Glc^{III} α: 4.93, IdoUA^{IV}: 5.30, Glc^{V} α: 5.02 and IdoUA^{VI}: 5.09.

### 5-Phenylpentyl (methyl 2-O-acetyl-3-O-methyl-α-L-idopyranosyluronate)-(1→4)-(2-azido-2-deoxy-3-O-methyl-6-O-acetyl-α-D-glucopyranosyl)-(1→4)-(methyl 2-O-acetyl-3-O-methyl-α-L-idopyranosyluronate)-(1→4)-(2-azido-2-deoxy-3-O-methyl-6-O-tert-butyldiphenylsilyl-α-D-glucopyranosyl)-(1→4)-(methyl 2-O-acetyl-3-O-methyl-α-L-idopyranosyluronate)-(1→4)-2-[(benzyloxy)carbonyl]amino-2-deoxy-3-O-methyl-6-O-tert-butyldiphenylsilyl-β-D-glucopyranoside (76)

Compound 75 (1.74 g, 0.78 mmol) is processed according to the same procedure as that described for the preparation of 33, to give compound 76 (1.53 g).

¹H NMR [500 MHz] (CDCl₃) δ of the anomeric protons Glc^{I} β: 4.54, IdoUA^{II}: 5.17, Glc^{III} α: 4.92, IdoUA^{IV}: 5.30, Glc^{V} α: 5.01 and IdoUA^{VI}: 5.01.

### 5-Phenylpentyl (methyl 2-O-acetyl-4-O-benzyl-3-O-methyl-α-L-idopyranosyluronate)-(1→4)-(2-azido-2-deoxy-3-O-methyl-6-O-acetyl-α-D-glucopyranosyl)-(1→4)-(methyl 2-O-acetyl-3-O-methyl-α-L-idopyranosyluronate)-(1→4)-(2-azido-2-deoxy-3-O-methyl-6-O-acetyl-α-D-b lucopyranosyl)-(1→4)-(methyl 2-O-acetyl-3-O-methyl-α-L-idopyranosyluronate)-(1→4)-(2-azido-2-deoxy-3-O-methyl-6-O-tert-butyldiphenylsilyl-α-D-glucopyranosyl)-(1→4)-(methyl 2-O-acetyl-3-O-methyl-α-L-idopyranosyluronate)-(1→4)-2-[(benzyloxy)carbonyl]amino-2-deoxy-3-O-methyl-6-O-tert-butyldiphenylsilyl-β-D-glucopyranoside (77)

Compound 76 (200.2 mg, 0.094 mmol) and compound 28 (90.5 mg, 0.122 mmol) are processed according to the same procedure as that described for the preparation of 34, to give compound 77 (626.3 mg).

¹H NMR [500 MHz] (CDCl₃) δ of the anomeric protons Glc^{I} β: 4.54 IdoUA^{II}: 5.17, Glc^{III} α: 4.93, IdoUA^{IV}: 5.29, Glc^{V} α: 5.01, IdoUA^{VI}: 5.10, Glc^{VII} α: 4.99 and IdoUA^{VIII} 5.14.

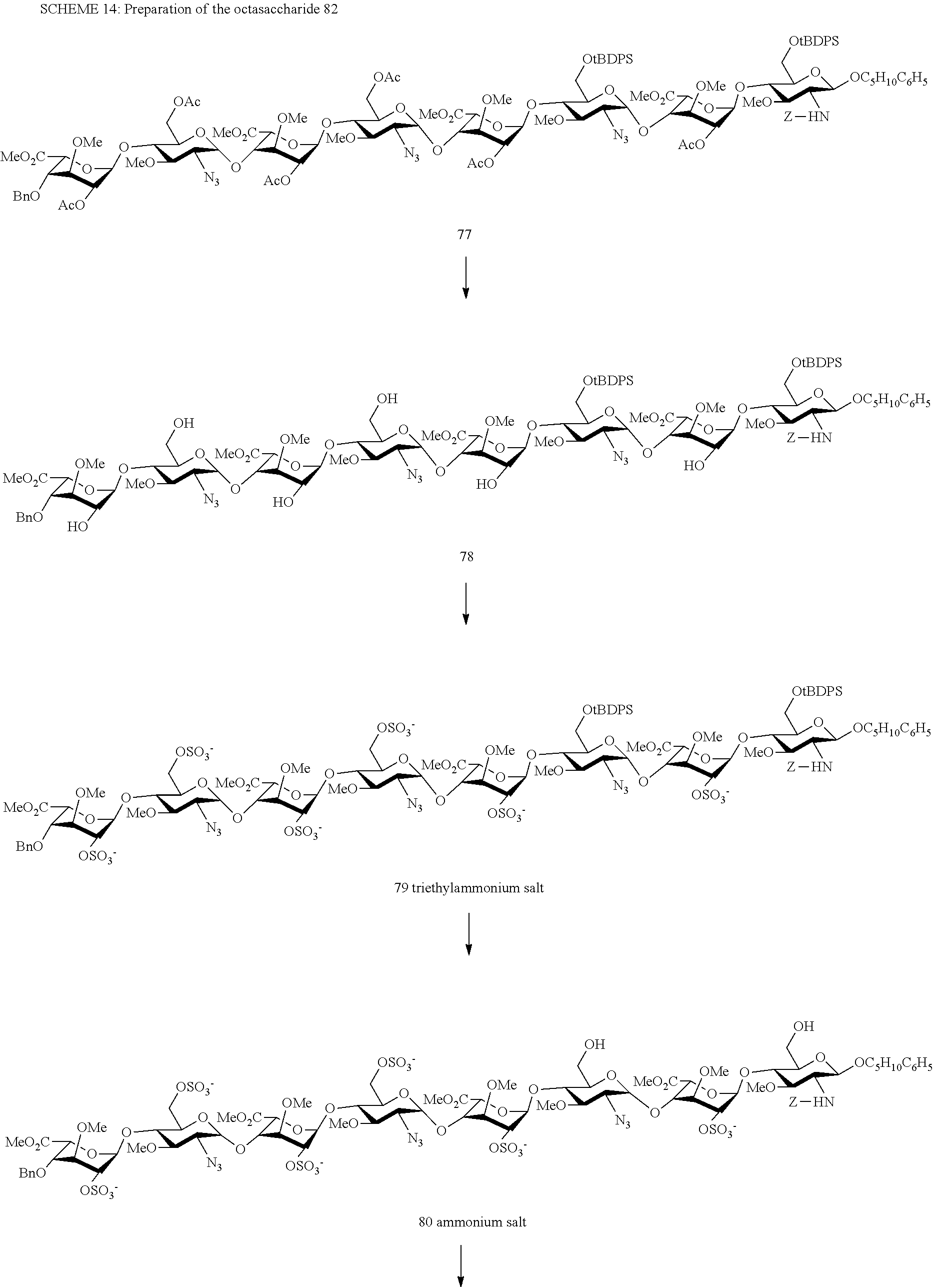

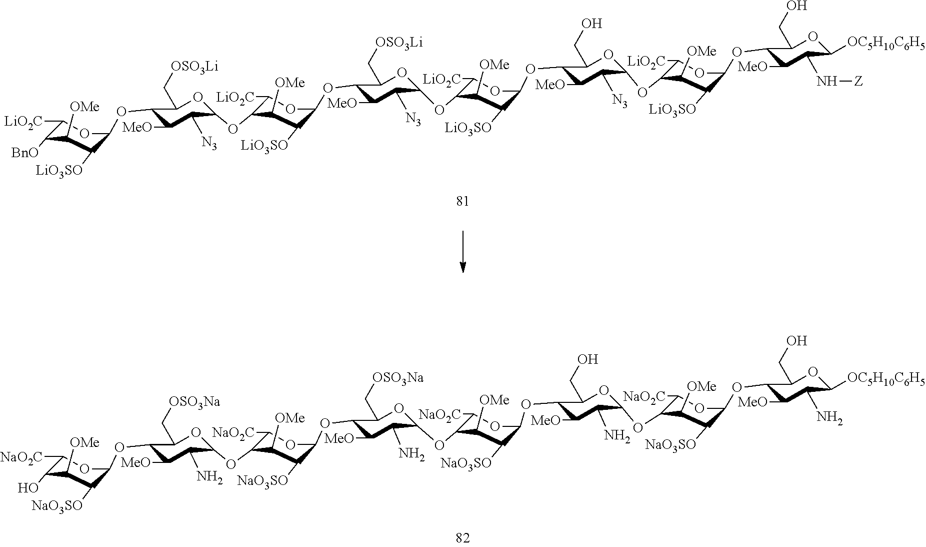

### 5-Phenylpentyl (methyl 4-O-benzyl-3-O-methyl-α-L-idopyranosyluronate)-(1→4)-(2-azido-2-deoxy-3-O-methyl-α-D-glucopyranosyl)-(1→4)-(methyl 3-O-methyl-α-L-idopyranosyluronate)-(1→4)-(2-azido-2-deoxy-3-O-methyl-α-D-glucopyranosyl)-(1→4)-(methyl 3-O-methyl-α-L-idopyranosyluronate)-(1→4)-(2-azido-2-deoxy-3-O-methyl-6-O-tert-butyldiphenylsilyl-α-D-glucopyranosyl)-(1→4)-(methyl 3-O-methyl-α-L-idopyranosyluronate)-(1→4)-2-[(benzyloxy)carbonyl]amino-2-deoxy-3-O-methyl-6-O-tert-butyldiphenylsilyl-β-D-glucopyranoside (78)

Compound 77 (130 mg, 0.0479 mmol) is processed according to the same procedure as that described for the preparation of 35, to give compound 78 (106 mg).

¹H NMR [500 MHz] (CDCl₃) δ of the anomeric protons Glc^{I} β: 4.56, IdoUA^{II}: 5.24, Glc^{III} α: 5.06, IdoUA^{IV}: 5.19, Glc^{V} α: 5.07, IdoUA^{VI}: 5.20, Glc^{VII} α: 5.00 and IdoUA^{VIII}: 5.11.

### 5-Phenylpentyl (methyl 4-O-benzyl-3-O-methyl-2-O-triethylammonium sulphonato-α-L-idopyranosyluronate)-(1→4)-(2-azido-2-deoxy-3-O-methyl-6-O-triethylammonium sulphonato-α-D-glucopyranosyl)-(1→4)-(methyl 3-O-methyl-2-O-triethylammonium sulphonato-α-L-idopyranosyluronate)-(1→4)-(2-azido-2-deoxy-3-O-methyl-6-O-triethylammonium sulphonato-α-D-glucopyranosyl)-(1→4)-(methyl 3-O-methyl-2-O-triethylammonium sulphonato-α-L-idopyranosyluronate)-(1→4)-(2-azido-2-deoxy-3-O-methyl-6-O-tert-butyldiphenylsilyl-α-D-glucopyranosyl)-(1→4)-(methyl 3-O-methyl-2-O-triethylammonium sulphonato-α-L-idopyranosyluronate)-(1→4)-2-[(benzyloxy)carbonyl]amino-2-deoxy-3-O-methyl-6-O-tert-butyldiphenylsilyl-β-D-glucopyranoside (79)

Compound 78 (205 mg, 0.0833 mmol) is processed according to the same procedure as that described for the preparation of 36, to give compound 79 (234.1 mg).

¹H NMR [500 MHz] (CDCl₃) δ of the anomeric protons Glc^{I} β: 4.31, IdoUA^{II}: 5.36, Glc^{III} α: 5.26, IdoUA^{IV}: 5.39, Glc^{V} α: 5.25, IdoUA^{VI}: 5.34, Glc^{VII} α: 5.18 and IdoUA^{VIII}: 5.34.

### 5-Phenylpentyl (methyl 4-O-benzyl-3-O-methyl-2-O-ammonium sulphonato-α-L-idopyranosyluronate)-(1→4)-(2-azido-2-deoxy-3-O-methyl-6-O-ammonium sulphonato-α-D-glucopyranosyl)-(1→4)-(methyl 3-O-methyl-2-O-ammonium sulphonato-α-L-idopyranosyluronate)-(1→4)-(2-azido-2-deoxy-3-O-methyl-6-O-ammonium sulphonato-α-D-glucopyranosyl)-(1→4)-(methyl 3-O-methyl-2-O-ammonium sulphonato-α-L-idopyranosyluronate)-(1→4)-(2-azido-2-deoxy-3-O-methyl-α-D-glucopyranosyl)-(1→4)-(methyl 3-O-methyl-2-O-ammonium sulphonato-α-L-idopyranosyluronate)-(1→4)-2-[(benzyloxy)carbonyl]amino-2-deoxy-3-O-methyl-β-D-glucopyranoside (80)

Ammonium fluoride (221 mg, 80 molar equivalents) is added to a solution of compound 79 (230 mg, 0.0748 mmol) previously obtained in methanol (9.7 ml). After magnetic stirring at 55° C. for 20 h, the reaction mixture is purified using a Sephadex® G25-fine gel column (800 ml) eluted with a 0.2 M aqueous solution of NaCl. The fractions containing the expected compound are combined, and loaded onto a Sephadex® G25-fine gel column (800 ml) eluted with water. The fractions containing the product are then concentrated under strong vacuum, to give compound 80 (195.7 mg).

¹H NMR [500 MHz] (CD₃OD) δ of the anomeric protons Glc^{I} β: 4.51 IdoUA^{II}: 5.26, Glc^{III} α: 5.38, IdoUA^{IV}: 5.27, Glc^{V} α: 5.38, IdoUA^{VI}: 5.26, Glc^{VII} α: 5.38 and IdoUA^{VIII}: 5.23.

### 5-Phenylpentyl (lithium 4-O-benzyl-3-O-methyl-2-O-lithium sulphonato-α-L-idopyranosyluronate)-(1→4)-(2-azido-2-deoxy-3-O-methyl-6-O-lithium sulphonato-α-D-glucopyranosyl)-(1→4)-(lithium 3-O-methyl-2-O-lithium sulphonato-α-L-idopyranosyluronate)-(1→4)-(2-azido-2-deoxy-3-O-methyl-6-O-lithium sulphonato-α-D-glucopyranosyl)-(1→4)-(lithium 3-O-methyl-2-O-lithium sulphonato-α-L-idopyranosyluronate)-(1→4)-(2-azido-2-deoxy-3-O-methyl-α-D-glucopyranosyl)-(1→4)-(lithium 3-O-methyl-2-O-lithium sulphonato-α-L-idopyranosyluronate)-(1→4)-2-[(benzyloxy)carbonyl]amino-2-deoxy-3-O-methyl-β-D-glucopyranoside (81)

Compound 80 (193 mg, 0.0743 mmol) is processed according to the same procedure as that described for the preparation of 37, to give compound 81 (178.5 mg).

¹H NMR [600 MHz] (CD₃OD) δ of the anomeric protons Glc^{I} β: 4.54, IdoUA^{II}: 5.20, Glc^{III} α: 5.32, IdoUA^{IV}: 5.24, Glc^{V} α: 5.33, IdoUA^{VI}: 5.23, Glc^{VII} α: 5.34 p and IdoUA^{VIII}: 5.18.

### 5-Phenylpentyl (sodium 3-O-methyl-2-O-sodium sulphonato-α-L-idopyranosyluronate)-(1→4)-(2-amino-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1>4)-(2-amino-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-amino-2-deoxy-3-O-methyl-α-D-glucopyranosyl)-(1→4)-(sodium 3-O-methyl-2-O-sodium sulphonato-α-L-idopyranosyluronate)-(1→4)-2-amino-2-deoxy-3-O-methyl-β-D-glucopyranoside (82)

Compound 81 (23 mg, 0.00875 mmol) is processed according to the same procedure as that described for the preparation of 38, to give compound 82 (16.2 mg).

¹H NMR [600 MHz] (CD₃OD) δ of the anomeric protons Glc^{I} β: 4.48, IdoUA^{II}: 5.34, Glc^{III} α: 5.46, IdoUA^{IV}: 5.31, Glc^{V} α: 5.46, IdoUA^{VI}: 5.26, Glc^{VII} α: 5.46 p and IdoUA^{VIII}: 5.23.

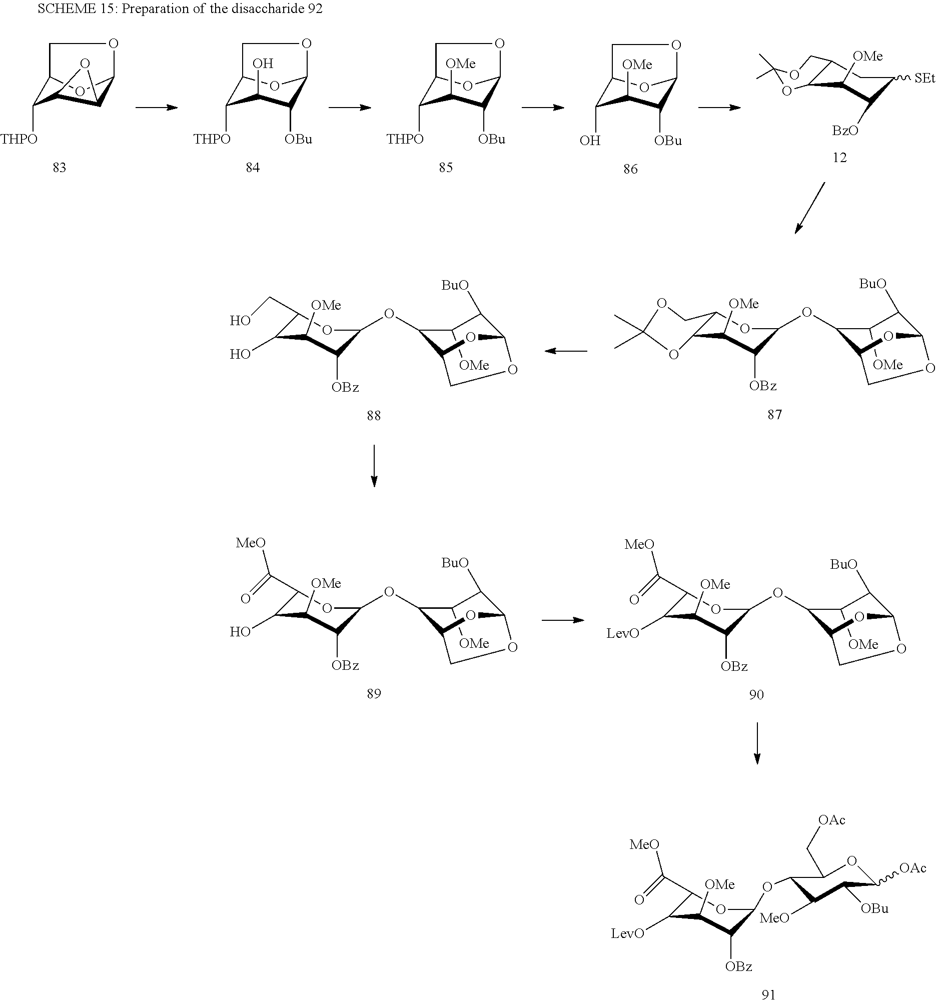

### 1,6-Anhydro-2-O-butyl-4-O-tetrahydropyranyl-β-D-glucopyranose (84)

Butan-1-ol (16.1 ml, 176 mmol), dropwise, and then 55% sodium hydride (3.5 g, 88 mmol), in several fractions, are successively added at 0° C. to a solution of compound 83 (2 g, 8.8 mmol, described in Carbohydrate Research, 64 (1978) 339-364) in ethylene glycol dimethyl ether (88 ml). At the end of the addition, the temperature is gradually increased to 85° C. and the reaction mixture is stirred magnetically for 5 h 15 min. The mixture is then diluted at 0° C. with ethyl acetate. The organic phase is washed with water, dried over sodium sulphate, filtered and then evaporated under vacuum. The residue is purified by chromatography on a silica gel column (toluene/acetone), to give 1.81 g of compound 84.

¹H NMR [500 MHz] (CDCl₃) δ of the anomeric proton: 5.5 Glc^{I}.

### 1,6-Anhydro-2-O-butyl-3-O-methyl-4-O-tetrahydropyranyl-β-D-glucopyranose (85)

55% sodium hydride (400 mg, 10 mmol) is added at 0° C. to a solution of compound 84 (2.02 g, 6.7 mmol) in N,N-dimethylformamide (67 ml). After stirring at ambient temperature for 20 min, iodomethane (830 μl, 13.4 mmol) is added dropwise at 0° C. After stirring at ambient temperature for 1 h, methanol (1.7 ml) is added at 0° C. and, after stirring at ambient temperature for 1 h, the mixture is concentrated under vacuum. The compound obtained is used in the next step without purification or characterization.

### 1,6-Anhydro-2-O-butyl-3-O-methyl-β-D-glucopyranose (86)

The residue previously obtained is dissolved in methanol (37 ml) and then a 1M aqueous solution of hydrochloric acid (7.4 ml) is added dropwise at 0° C. After stirring at ambient temperature for 1 h 30 min, a 1M aqueous solution of sodium hydroxide (7 ml) is added at 0° C., and then the mixture is concentrated under vacuum. The residue obtained is purified by chromatography on a silica gel column (toluene/acetone), to give 1.37 g of compound 86.

SFC-MS m/z 255 [(M+Na)⁺]. T_{R}=8.21 min

### (2-O-Benzoyl-4,6-O-isopropylidene-3-O-methyl-α-L-idopyranosyl)-(1→4)-1,6-anhydro-2-O-butyl-3-O-methyl-β-D-glucopyranose (87)

A mixture of thioglycoside 12 (2.9 g, 7.7 mmol), of the glycosyl acceptor 86 (1.37 g, 5.9 mmol) and of 4 Å molecular sieve powder (3.9 g) in dichloromethane (88 ml) is stirred under an argon atmosphere for 1 h 30 min at ambient temperature. The reaction mixture is cooled to −20° C. and N-iodosuccinimide (1.85 g, 8.26 mmol), in solution in a 1/1 dioxane/dichloromethane mixture (30 ml), and a 1M solution of triflic acid in a 1/1 dioxane/dichloromethane mixture (1.16 ml) are successively added. After stirring for 15 min, the reaction medium is neutralized by adding solid sodium hydrogen carbonate and then filtered through Celite®. The filtrate is then washed with a saturated solution of sodium thiosulphate. The organic phase is dried over sodium sulphate, filtered and then evaporated under vacuum. The residue is purified by chromatography on a silica gel column (heptane/ethyl acetate), to give 2.36 g of compound 87.

¹H NMR [500 MHz] (CDCl₃) δ of the anomeric protons: IdoUA^{II} 5.22 and Glc^{I}: 5.38.

### (2-O-Benzoyl-3-O-methyl-α-L-idopyranosyl)-(1→4)-1,6-anhydro-2-O-butyl-3-O-methyl-β-D-glucopyranose (88)

Aqueous acetic acid (70%) (8.6 ml) is added at ambient temperature to a solution of compound 87 (2.36 g, 4.3 mmol) in 1,2-dichloroethane (1.7 ml). After stirring at 60° C. for 2 h, the reaction medium is concentrated under vacuum. The residue is coevaporated with toluene and then purified by chromatography on a silica gel column (toluene/acetone), to give 2.06 g of compound 88.

¹H NMR [500 MHz] (CDCl₃) δ of the anomeric protons: IdoUA^{II} 5.21 and Glc^{I}: 5.43.

### (Methyl 2-O-benzoyl-3-O-methyl-α-L-idopyranosyluronate)-(1→4)-1,6-anhydro-2-O-butyl-3-O-methyl-β-D-glucopyranose (89)

A solution of 2,2,6,6-tetramethylpiperidin-1-oxy (13 mg, 0.0804 mmol) in tetrahydrofuran (270 μl) and a solution of 1,3-dibromo-5,5-dimethylhydantoin (2.3 g, 8.04 mmol) in tetrahydrofuran (6.9 ml) are added successively, at 0° C., to a solution of compound 88 (2.06 g, 4.02 mmol) in tetrahydrofuran (14.1 ml) and of saturated sodium hydrogen carbonate (16.1 ml). After stirring at ambient temperature for 3 h 15 min, the reaction medium is concentrated. The residue is coevaporated with N,N-dimethyl-formamide and the compound obtained is used in the next step without purification. The residue obtained is dissolved in N,N-dimethylformamide (28 ml) and then solid potassium hydrogen carbonate (2.0 g) and iodomethane (2.5 ml) are added at 0° C. After magnetic stirring at ambient temperature for 16 h, the reaction mixture is concentrated. The residue obtained is dissolved in dichloromethane and is then washed with a saturated aqueous solution of sodium thiosulphate, dried over sodium sulphate, filtered and then evaporated under vacuum. A brief purification was carried out (toluene/acetone). Compound 89 was obtained with sufficient purity to be used in the next step.

¹H NMR [500 MHz] (CDCl₃) δ of the anomeric protons Ido: IdoUA^{II} 5.18 and Glc^{I}: 5.32.

### (Methyl 2-O-benzoyl-4-O-levulinoyl-3-O-methyl-α-L-idopyranosyluronate)-(1→4)-1,6-anhydro-2-O-butyl-3-O-methyl-β-D-glucopyranose (90)

4-dimethylaminopyridine (98 mg, 0.804 mmol), 1-(3-dimethylaminopropyl)-3-ethylcarbodiimide hydrochloride (1.5 g, 8.04 mmol) and levulinic acid (827 μl, 8.04 mmol) are added successively to a solution of compound 89 in dioxane (48.2 ml). After stirring at ambient temperature for 16 h, the reaction mixture is diluted with dichloromethane. The organic phase is washed successively with a 10% aqueous solution of potassium hydrogen sulphate, a saturated aqueous solution of sodium hydrogen carbonate and a saturated aqueous solution of sodium chloride, and then dried over sodium sulphate, filtered and evaporated to dryness. A brief purification was carried out (toluene/acetone). Compound 90 was obtained with sufficient purity to be used in the next step.

¹H NMR [500 MHz] (CDCl₃) δ of the anomeric protons Ido: IdoUA^{II} 5.31 and Glc^{I}: 5.4.

### (Methyl 2-O-benzoyl-4-O-levulinoyl-3-O-methyl-α-L-idopyranosyluronate)-(1→4)-1,6-di-O-acetyl-2-O-butyl-3-O-methyl-α,β-D-glucopyranose (91)

Trifluoroacetic acid (3.5 ml, 44.2 mmol) is added, at 0° C., to a solution of compound 90 in acetic anhydride (38 ml). The reaction medium is stirred for 16 h at ambient temperature. After concentration, the mixture is coevaporated with toluene. Purification of the residue by chromatography on a silica gel column (toluene/acetone) gives 2.4 g of compound 91.

Rf=0.48, silica gel, 4/1 v/v toluene/acetone

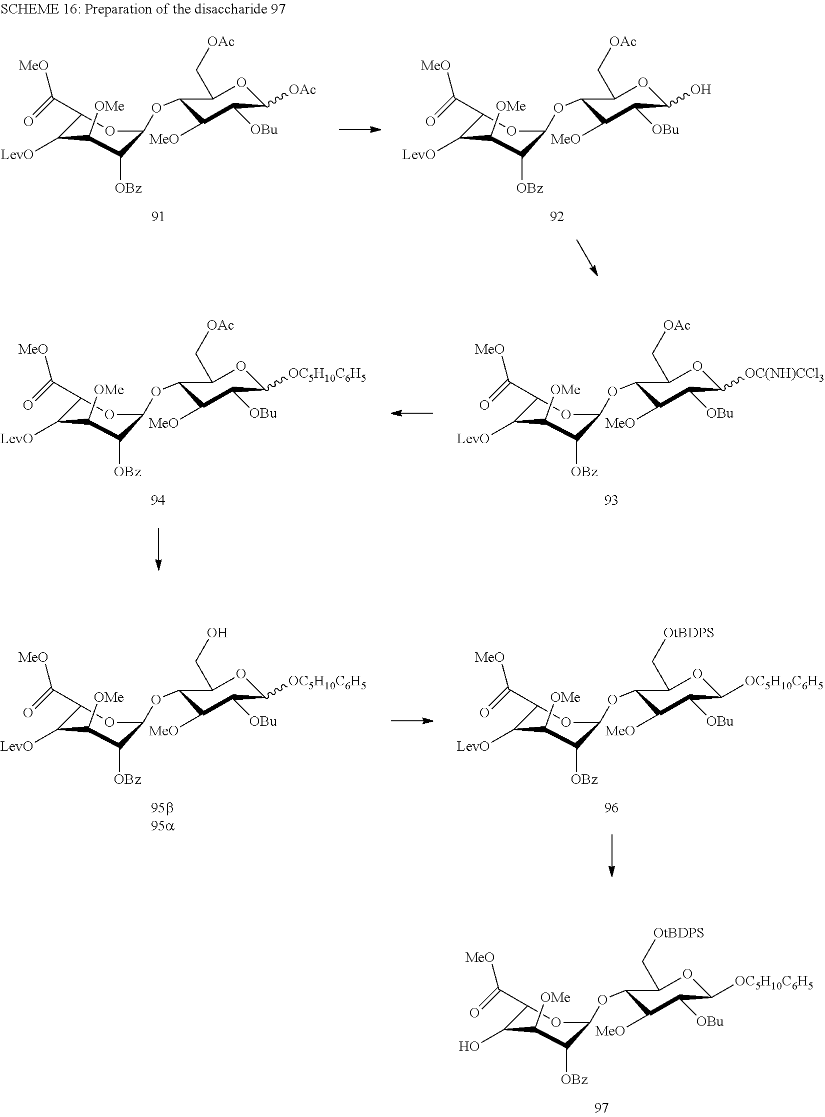

### (Methyl 2-O-benzoyl-4-O-levulinoyl-3-O-methyl-α-L-idopyranosyluronate)-(1→4)-6-O-acetyl-2-O-butyl-3-O-methyl-α,β-D-glucopyranose (92)

Acetic acid (8.7 μl, 0.15 mmol) and then morpholine (2.7 ml, 30.5 mmol) are added, at 0° C., to a solution of compound 91 (2.26 g, 3.05 mmol) in toluene (6.1 ml). After stirring at ambient temperature for 6 h 15 min, the progression of the reaction is stopped by adding, at 0° C., a 1M aqueous solution of hydrochloric acid (31.5 ml). The aqueous phase is extracted with ethyl acetate. The combined organic phases are dried over sodium sulphate, filtered and then concentrated to dryness. The residue is chromatographed on a silica gel column (toluene/acetone), to give 2.0 g of compound 92.

Rf=0.26, silica gel, 4/1 v/v toluene/acetone.

### (Methyl 2-O-benzoyl-4-O-levulinoyl-3-O-methyl-α-L-idopyranosyluronate)-(1→4)-6-O-acetyl-2-O-butyl-3-O-methyl-α,β-D-glucopyranose trichloroacetimidate (93)

Trichloroacetonitrile (1.4 ml, 14.3 mmol) and caesium carbonate (1.49 g, 4.6 mmol) are added, at 0° C., to a solution of compound 92 (2.0 g, 2.86 mmol) in dichloromethane (54 ml). After stirring at ambient temperature for 1 h 30 min, the reaction medium is filtered through Celite® and then concentrated. The residue is purified by chromatography on a silica gel column (toluene/acetone+0.1% triethylamine), to give 2.17 g of compound 93.

Rf=0.46, silica gel, 4/1 v/v toluene/acetone

### 5-Phenylpentyl (methyl 2-O-benzoyl-4-O-levulinoyl-3-O-methyl-α-L-idopyranosyluronate)-(1→4)-6-O-acetyl-2-O-butyl-3-O-methyl-α,β-D-glucopyranoside (94)

A mixture of trichloroacetimidate 93 (4.73 g, 5.6 mmol), of 5-phenylpentan-1-ol (4.7 ml, 28 mmol) and of 4 Å molecular sieve powder (7.3 g) in dichloromethane (252 ml) is stirred under an argon atmosphere for 1 h 30 min at ambient temperature. The reaction mixture is cooled to −20° C. and tert-butyldimethylsilyl triflate (296 μl, 1.12 mmol) is added dropwise. After stirring at −20° C. for 45 min, the progression of the reaction is stopped by adding solid sodium hydrogen carbonate. The reaction medium is filtered through Celite® and then the filtrate is washed with a 2% aqueous solution of sodium hydrogen carbonate. The organic phase is dried over sodium sulphate, filtered and then evaporated under vacuum. The residue is purified by chromatography on a silica gel column (cyclohexane/acetone), to give 4.55 g of compound 94.

¹H NMR [500 MHz] (CDCl₃) δ of the anomeric protons IdoUA^{II} 5.19 ppm, Glc^{I}α: 4.86 ppm and Glc^{I}β: 4.23 ppm.

### 5-Phenylpentyl (methyl 2-O-benzoyl-4-O-levulinoyl-3-O-methyl-α-L-idopyranosyluronate)-(1→4)-2-O-butyl-3-O-methyl-β-D-glucopyranoside (95β) and 5-phenylpentyl (methyl 2-O-benzoyl-4-O-levulinoyl-3-O-methyl-α-L-idopyranosyluronate)-(1→4)-2-O-butyl-3-O-methyl-α-D-glucopyranoside (95α)

[tBu₂SnCl(OH)]₂ (220 mg, 0.773 mmol), prepared according to A. Orita et al., Chem. Eur. J. (2001) 7, 3321, is added, at ambient temperature under an argon atmosphere, to a solution of compound 94 (4.35 g, 5.15 mmol) in a 1/1 methanol/tetrahydrofuran mixture (62 ml). After magnetic stirring at ambient temperature for 40 h, the reaction medium is concentrated under vacuum and then the residue is purified by chromatography on a silica gel column (cyclohexane/acetone), to give 1.44 g of compound 95β, 1.02 g of compound 95α and 580 mg of 95α/β mixture.

Rf (95β)=0.25 and (95α) 0.13, silica gel, 4/1 v/v diisopropyl ether/ethyl acetate

### 5-Phenylpentyl (methyl 2-O-benzoyl-4-O-levulinoyl-3-O-methyl-α-L-idopyranosyluronate)-(1→4)-2-O-butyl-3-O-methyl-6-O-tert-butyldiphenylsilyl-β-D-glucopyranoside (96)

Triethylamine (345 μl, 2.5 mmol), 4-dimethylaminopyridine (61 mg, 0.5 mmol) and tert-butyldiphenylsilyl chloride (520 μl, 2.0 mmol) are added, under an argon atmosphere at 0° C., to compound 95β (800 mg, 1.0 mmol) dissolved in dichloromethane. The reaction medium is stirred at ambient temperature for 22 h and then tert-butyldiphenylsilyl chloride (130 μl, 0.5 mmol) is again added. After stirring at ambient temperature for 3 days, the progression of the reaction is stopped by adding methanol (122 μl, 2.75 mmol). After magnetic stirring for 30 min, the organic phase is washed with a 10% solution of potassium hydrogen sulphate, dried over sodium sulphate, filtered and then evaporated under vacuum. The residue is briefly purified by flash chromatography on a silica gel column (cyclohexane/acetone+0.1% triethylamine). Compound 96 is obtained with sufficient purity to be used in the next step.

Rf=0.29, silica gel, cyclohexane/acetone 3/1 v/v+0.1% triethylamine.

### 5-Phenylpentyl (methyl 2-O-benzoyl-3-O-methyl-α-L-idopyranosyluronate)-(1→4)-2-O-butyl-3-O-methyl-6-O-tert-butyldiphenylsilyl-β-D-glucopyranoside (97)

Hydrazine acetate (460 mg, 5.0 mmol) is added to a solution of compound 96 in a 1/2 toluene/ethanol mixture (200 ml). The reaction medium is stirred for 2 h at ambient temperature. After concentration under vacuum, the residue is taken up in dichloromethane and then washed with water. After drying over sodium sulphate, filtration and then concentration, the residue is chromatographed on a silica gel column (cyclohexane/acetone+0.1% triethylamine), to give 850 mg of compound 97.

Rf=0.28, silica gel, cyclohexane/acetone 3/1 v/v+0.1% triethylamine.

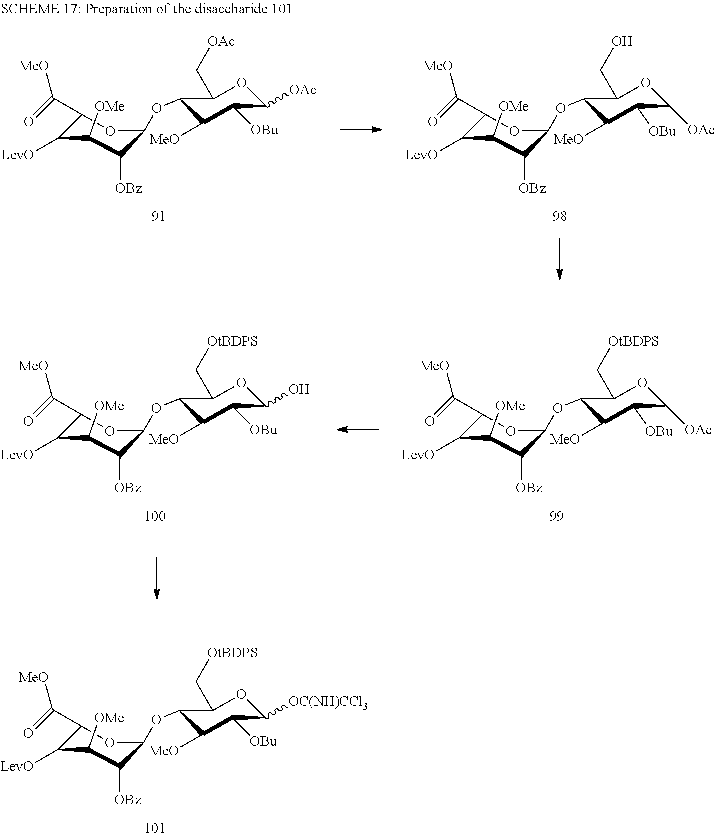

### (Methyl 2-O-benzoyl-4-O-levulinoyl-3-O-methyl-α-L-idopyranosyluronate)-(1→4)-1-O-acetyl-2-O-butyl-3-O-methyl-α-D-glucopyranose (98)

[tBu₂SnCl(OH)]₂ (610 mg, 2.39 mmol), prepared according to A. Orita et al., Chem. Eur. J. (2001) 7, 3321, is added to a solution of compound 91 (11.8 g, 15.94 mmol) in a 1/1 methanol/tetrahydrofuran mixture (191 ml). After magnetic stirring at ambient temperature for 8 h 30 min, the reaction mixture is concentrated under vacuum and then purified by chromatography on a silica gel column (toluene/acetone), to give compound 98 (8.26 g).

Rf=0.27, silica gel, 4/1 v/v toluene/acetone

### (Methyl 2-O-benzoyl-4-O-levulinoyl-3-O-methyl-α-L-idopyranosyluronate)-(1→4)-1-O-acetyl-2-O-butyl-3-O-methyl-6-O-tert-butyldiphenylsilyl-α-D-glucopyranose (99)

Compound 98 (8.26 g, 11.82 mmol) is placed in solution in dichloromethane (95 ml). 4-dimethylaminopyridine (722 mg, 5.91 mmol), triethylamine (4.1 ml, 29.55 mmol), and tert-butyldiphenylsilyl chloride (6.1 ml, 23.6 mmol) are successively added at 0° C. and under argon. After stirring at ambient temperature for 21 h, the progression of the reaction is stopped by adding methanol (1.2 ml, 26 mmol). After magnetic stirring for 1 h, the organic phase is washed with a 10% solution of potassium hydrogen sulphate, dried over sodium sulphate, filtered and then evaporated. The residue is purified by flash chromatography on a silica gel column (toluene/acetone+0.1% triethylamine), to give 10.13 g of compound 99.

¹H NMR [500 MHz] (CD₃OD) δ of the anomeric protons: IdoUA^{II} 5.36 and Glc^{I}α 6.28 ppm.

### (Methyl 2-O-benzoyl-4-O-levulinoyl-3-O-methyl-α-L-idopyranosyluronate)-(1→4)-2-O-butyl-3-O-methyl-6-O-tert-butyldiphenylsilyl-α,β-D-glucopyranose (100)

Acetic acid (9.4 μl, 0.165 mmol) and then morpholine (2.9 ml, 33 mmol) are added, at 0° C., to a solution of compound 99 (3.1 g, 3.3 mmol) in toluene (6.6 ml). After stirring at ambient temperature for 24 h, the reaction is stopped by adding, at 0° C., a 1M aqueous solution of hydrochloric acid (33.6 ml). The aqueous phase is extracted with ethyl acetate. The combined organic phases are dried over sodium sulphate, filtered and then concentrated to dryness. The residue is chromatographed on a silica gel column (toluene/acetone+0.1% triethylamine), to give 2.7 g of compound 100.

Rf=0.53 and 0.46, silica gel, toluene/acetone 4/1 v/v+0.1% triethylamine.

### (Methyl 2-O-benzoyl-4-O-levulinoyl-3-O-methyl-α-L-idopyranosyluronate)-(1→4)-2-O-butyl-3-O-methyl-6-O-tert-butyldiphenylsilyl-α,β-D-glucopyranose trichloroacetimidate (101)

Trichloroacetonitrile (1.5 ml, 15 mmol) and caesium carbonate (1.6 g, 4.8 mmol) are added, at 0° C., to a solution of compound 100 (2.7 g, 3 mmol) in dichloromethane (57 ml). After stirring at ambient temperature for 3 h, the reaction medium is filtered through Celite® and then concentrated under vacuum. The residue is purified by chromatography on a silica gel column (toluene/acetone+0.1% triethylamine), to give 3.18 g of compound 101.

¹H NMR [500 MHz] (CDCl₃) δ of the anomeric protons IdoUA^{II}: 5.34 ppm, Glc^{I}α: 6.48 ppm and Glc^{I}β: 5.66 ppm.

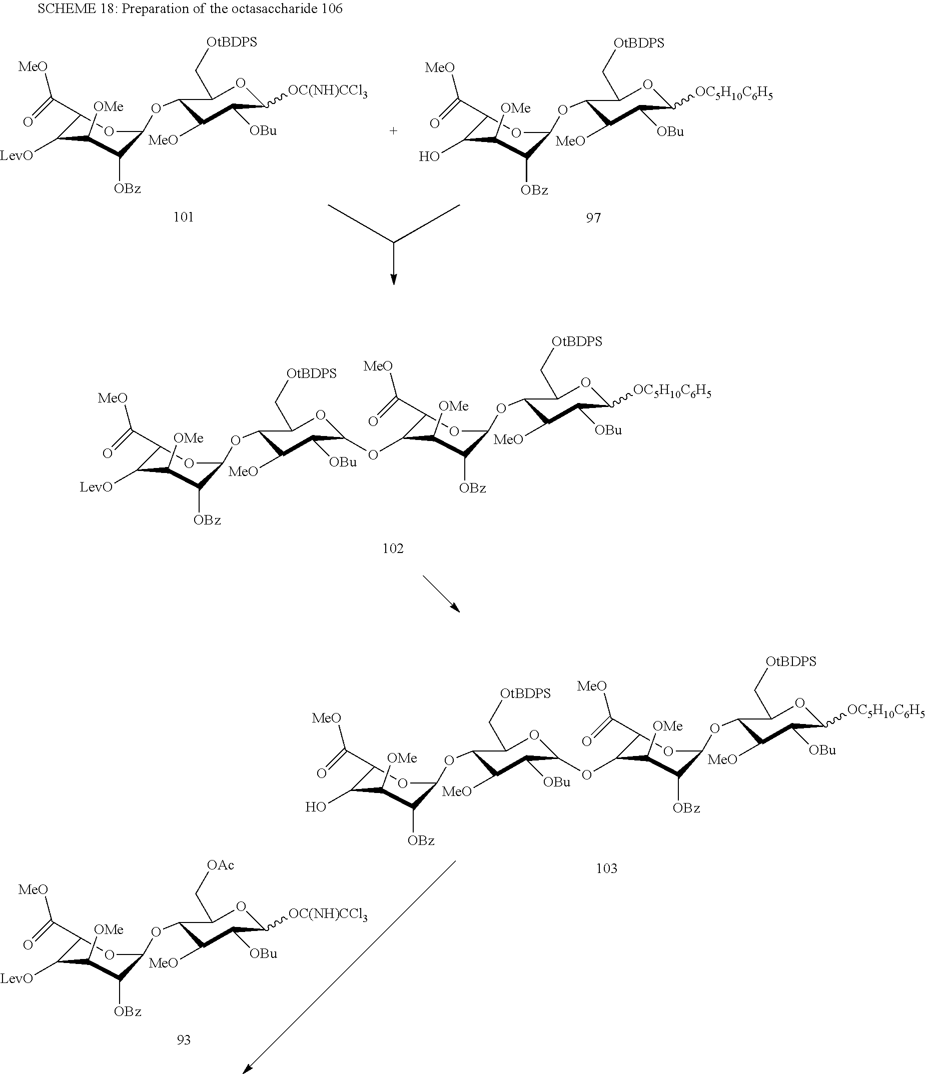

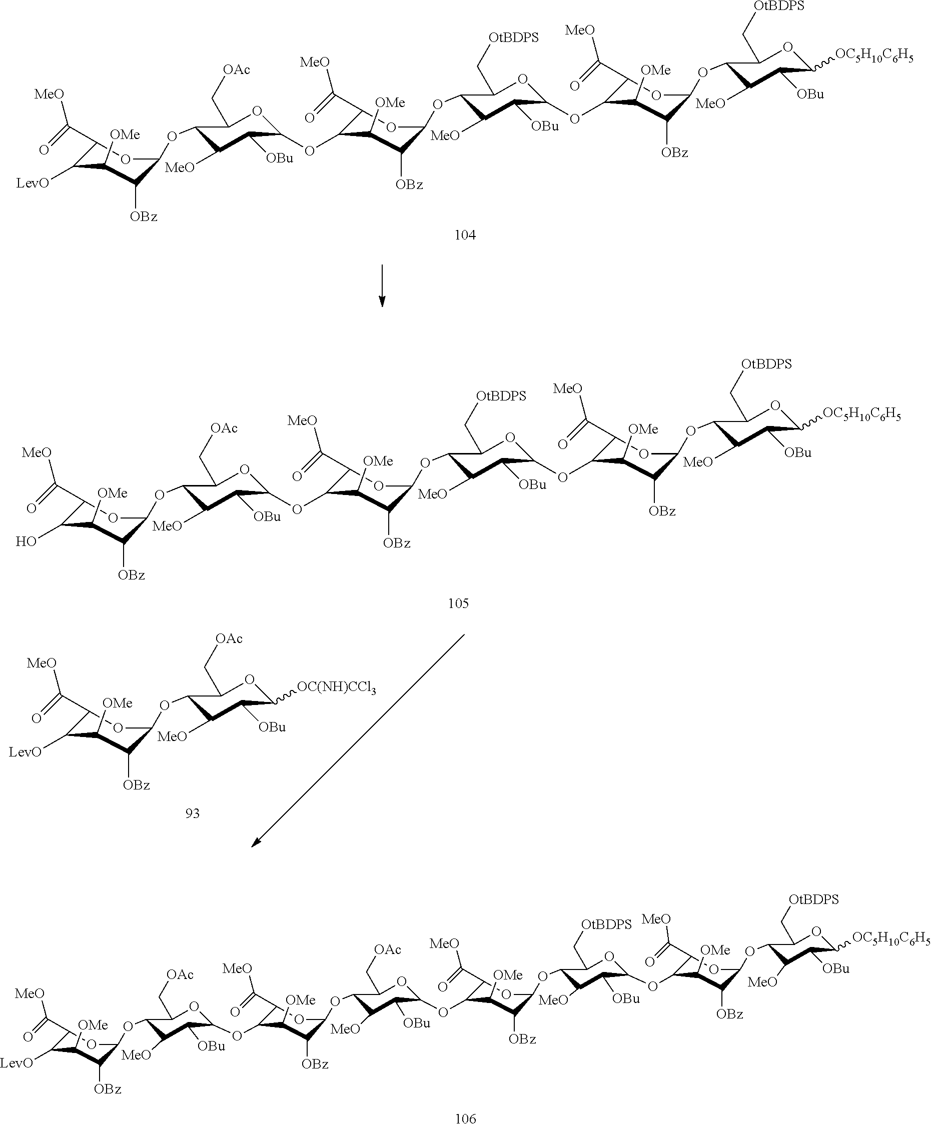

### 5-Phenylpentyl (methyl 2-O-benzoyl-4-O-levulinoyl-3-O-methyl-α-L-idopyranosyluronate)-(1→4)-(2-O-butyl-3-O-methyl-6-O-tert-butyldiphenylsilyl-α-D-glucopyranosyl)-(1→4)-(methyl 2-O-benzoyl-3-O-methyl-α-L-idopyranosyluronate)-(1→4)-2-O-butyl-3-O-methyl-6-O-tert-butyldiphenylsilyl-β-D-glucopyranoside (102)

A mixture of the glycosyl acceptor 97 (850 mg, 0.9 mmol), of compound 101 (1.15 g, 1.1 mmol) and of 4 Å molecular sieve powder (825 mg) in dichloromethane (39 ml) is stirred under an argon atmosphere for 1 h at ambient temperature. The reaction mixture is cooled to −20° C. and tert-butyldimethylsilyl triflate (38 μl, 0.165 mmol) is added. After stirring at −20° C. for 1 h, the reaction medium is neutralized by adding solid sodium hydrogen carbonate, and filtered through Celite®. The filtrate is washed with a 2% aqueous solution of sodium hydrogen carbonate. The organic phase is dried over sodium sulphate, filtered and then concentrated under vacuum. The residue obtained is chromatographed on a silica gel column (cyclohexane/acetone), to give 1.09 g of compound 102.

Rf=0.33, silica gel, 3/1 v/v cyclohexane/acetone

### 5-Phenylpentyl (methyl 2-O-benzoyl-3-O-methyl-α-L-idopyranosyluronate)-(1→4)-(2-O-butyl-3-O-methyl-6-O-tert-butyldiphenylsilyl-α-D-glucopyranosyl)-(1→4)-(methyl 2-O-benzoyl-3-O-methyl-α-L-idopyranosyluronate)-(1→4)-2-O-butyl-3-O-methyl-6-O-tert-butyldiphenylsilyl-β-D-glucopyranoside (103)

Hyrazine acetate (276 mg, 3.0 mmol) is added to a solution of compound 102 (1.09 g, 0.6 mmol) in a 1/2 toluene/ethanol mixture (120 ml). The reaction medium is stirred at ambient temperature for 2 h. After concentration under vacuum, the residue is placed in solution in dichloromethane and then washed with water. After drying over sodium sulphate, filtration and then concentration, the residue is purified on a silica gel column (cyclohexane/acetone), to give 1.02 g of compound 103.

¹H NMR [600 MHz] (CD₃OD) δ of the anomeric protons 5.37 IdoUA^{IV}, 4.83 Glc^{III}, 5.35 IdoUA^{II}, 4.18 Glc^{I}.

### 5-Phenylpentyl (methyl 2-O-benzoyl-4-O-levulinoyl-3-O-methyl-α-L-idopyranosyluronate)-(1→4)-(6-O-acetyl-2-O-butyl-3-O-methyl-α-D-glucopyranosyl)-(1→4)-(methyl 2-O-benzoyl-3-O-methyl-α-L-idopyranosyluronate)-(1→4)-(2-O-butyl-3-O-methyl-6-O-tert-butyldiphenylsilyl-α-D-glucopyranosyl)-(1→4)-(methyl 2-O-benzoyl-3-O-methyl-α-L-idopyranosyluronate)-(1→4)-2-O-butyl-3-O-methyl-6-O-tert-butyldiphenylsilyl-β-D-glucopyranoside (104)

A mixture of the glycosyl acceptor 103 (1.02 g, 0.592 mmol), of compound 93 (600 mg, 0.71 mmol) and of 4 Å molecular sieve powder (444 mg) in dichloromethane (20.7 ml) is stirred under an argon atmosphere for 1 h at ambient temperature. The reaction mixture is then cooled to −20° C. and tert-butyldimethylsilyl triflate (20.4 μl, 0.089 mmol) is added. After stirring at −20° C. for 1 h 15 min, the reaction medium is neutralized by adding solid sodium hydrogen carbonate, and is then filtered through Celite®. The filtrate is washed with a 2% aqueous solution of sodium hydrogen carbonate. The organic phase is dried over sodium sulphate, filtered and then concentrated under vacuum. The residue obtained is chromatographed on a silica gel column (cyclohexane/acetone), to give 1.05 g of compound 104.

Rf=0.31, silica gel, cyclohexane/acetone 7/3 v/v+0.1% triethylamine

### 5-Phenylpentyl (methyl 2-O-benzoyl-3-O-methyl-α-L-idopyranosyluronate)-(1→4)-(6-O-acetyl-2-O-butyl-3-O-methyl-α-D-glucopyranosyl)-(1→4)-(methyl 2-O-benzoyl-3-O-methyl-α-L-idopyranosyluronate)-(1→4)-(2-O-butyl-3-O-methyl-6-O-tert-butyldiphenylsilyl-α-D-glucopyranosyl)-(1→4)-(methyl 2-O-benzoyl-3-O-methyl-α-L-idopyranosyluronate)-(1→4)-2-O-butyl-3-O-methyl-6-O-tert-butyldiphenylsilyl-β-D-glucopyranoside (105)

Hydrazine acetate (201 mg, 2.18 mmol) is added to a solution of compound 104 (1.05 g, 0.44 mmol) in a 1/2 toluene/ethanol mixture (88 ml). After magnetic stirring for 1 h 30 min and concentration, the residue is placed in solution in dichloromethane and then washed with water. After drying over sodium sulphate, filtration and then concentration of the organic phase, the residue is purified on a silica gel column (cyclohexane/acetone), to give 975 mg of compound 105.

¹H NMR [600 MHz] (CDCl₃) δ of the anomeric protons: 5.12 IdoUA^{VI}, 4.89 Glc^{V}, 5.42 IdoUA^{IV}, 4.83 Glc^{III}, 5.34 IdoUA^{II}, 4.18 Glc^{I}.

### 5-Phenylpentyl (methyl 2-O-benzoyl-4-O-levulinoyl-3-O-methyl-α-L-idopyranosyluronate)-(1→4)-(6-O-acetyl-2-O-butyl-3-O-methyl-α-D-glucopyranosyl)-(1→4)-(methyl 2-O-benzoyl-3-O-methyl-α-L-idopyranosyluronate)-(1→4)-(6-O-acetyl-2-O-butyl-3-O-methyl-α-D-glucopyranosyl)-(1→4)-(methyl 2-O-benzoyl-3-O-methyl-α-L-idopyranosyluronate)-(1→4)-(2-O-butyl-3-O-methyl-6-O-tert-butyldiphenylsilyl-α-D-glucopyranosyl)-(1→4)-(methyl 2-O-benzoyl-3-O-methyl-α-L-idopyranosyluronate)-(1→4)-2-O-butyl-3-O-methyl-6-O-tert-butyl-diphenylsilyl-β-D-glucopyranoside (106)

A mixture of the glycosyl acceptor 105 (975 mg, 0.423 mmol), of compound 93 (535 mg, 0.634 mmol) and of 4 Å molecular sieve powder (317 mg) in toluene (15 ml) is stirred under an argon atmosphere for 1 h at ambient temperature. The reaction mixture is cooled to −20° C. and tert-butyldimethylsilyl triflate (15 μl, 0.089 mmol) is added. After stirring at −20° C. for 35 min, the reaction medium is neutralized by adding solid sodium hydrogen carbonate and filtered through Celite®. The filtrate is washed with a 2% aqueous solution of sodium hydrogen carbonate. The organic phase is dried over sodium sulphate, filtered and then concentrated. The residue obtained is chromatographed on a silica gel column (cyclohexane/acetone), to give 1.25 g of compound 106.

Rf=0.24, silica gel, 7/3 v/v cyclohexane/acetone

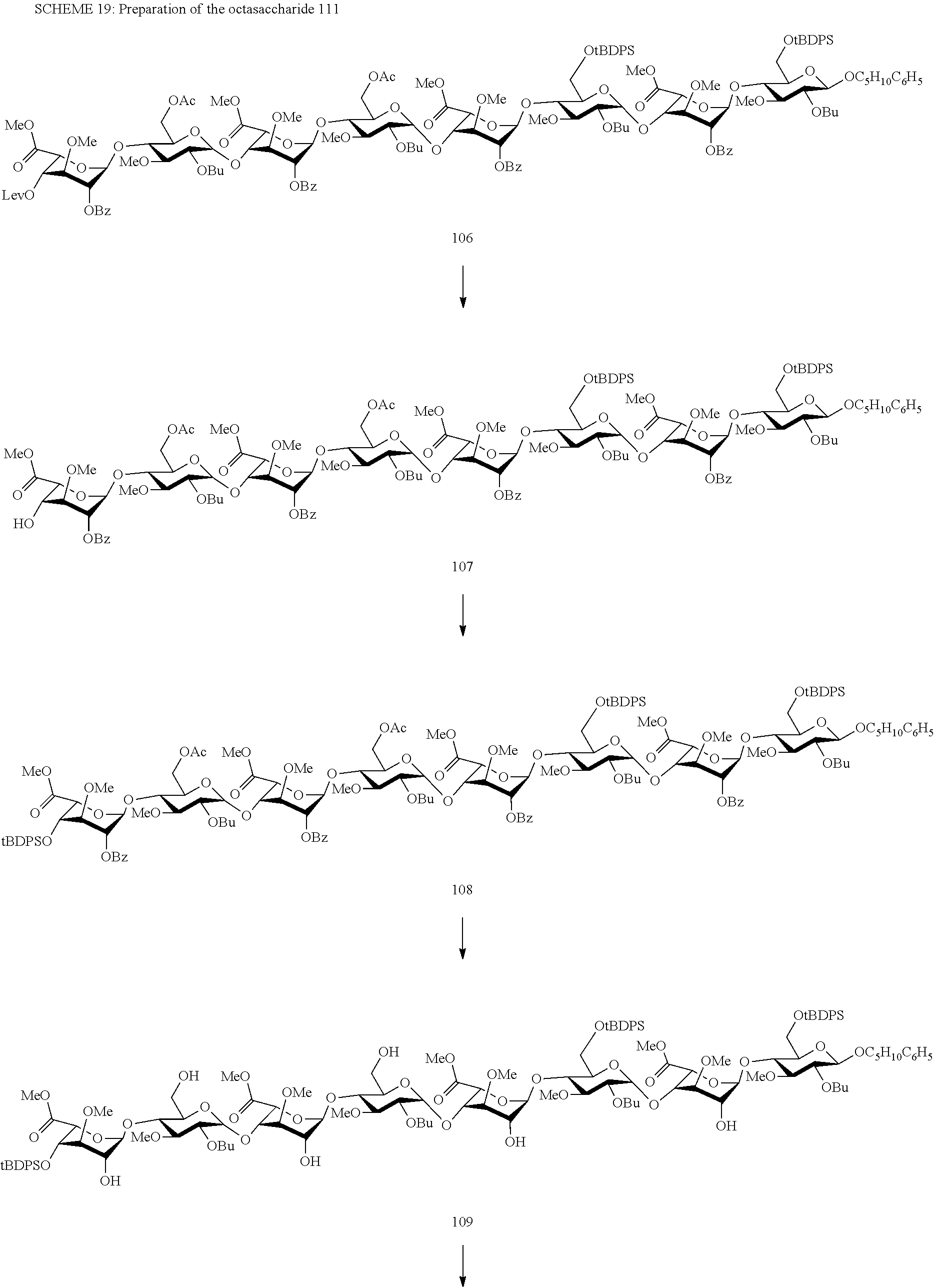

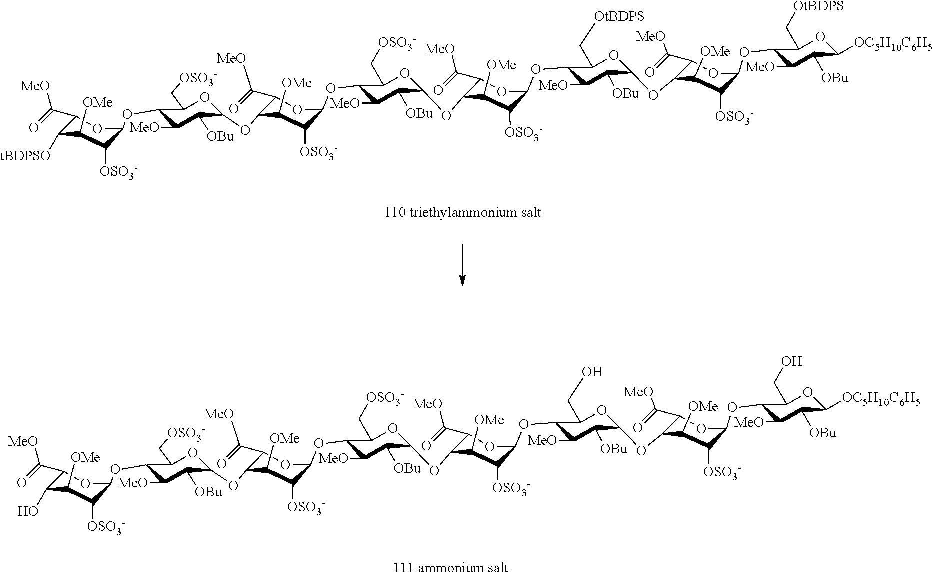

### 5-Phenylpentyl (methyl 2-O-benzoyl-3-O-methyl-α-L-idopyranosyluronate)-(1→4)-(6-O-acetyl-2-O-butyl-3-O-methyl-α-D-glucopyranosyl)-(1→4)-(methyl 2-O-benzoyl-3-O-methyl-α-L-idopyranosyluronate)-(1→4)-(6-O-acetyl-2-O-butyl-3-O-methyl-α-D-glucopyranosyl)-(1→4)-(methyl 2-O-benzoyl-3-O-methyl-α-L-idopyranosyluronate)-(1→4)-(2-O-butyl-3-O-methyl-6-O-tert-butyldiphenylsilyl-α-D-glucopyranosyl)-(1→4)-(methyl 2-O-benzoyl-3-O-methyl-α-L-idopyranosyluronate)-(1→4)-2-O-butyl-3-O-methyl-6-O-tert-butyldiphenylsilyl-(3-D-glucopyranoside (107)

Hydrazine acetate (193 mg, 2.09 mmol) is added to a solution of compound 106 (1.25 g, 0.419 mmol) in a 1/2 toluene/ethanol mixture (84 ml). The reaction medium is stirred at ambient temperature for 1 h 45 min. After concentration, the residue is taken up in dichloromethane and then washed with water. After drying over sodium sulphate, filtration and then concentration, the residue is purified on a silica gel column (cyclohexane/acetone), to give 0.99 g of compound 107.

SFC-MS m/z 1463 [(M+2H+CH₃CN)²⁺]/2. T_{R1}=9.23 min

### 5-Phenylpentyl (methyl 2-O-benzoyl-3-O-methyl-4-O-tert-butyldiphenylsilyl-α-L-idopyranosyluronate)-(1→4)-(6-O-acetyl-2-O-butyl-3-O-methyl-α-D-glucopyranosyl)-(1→4)-(methyl 2-O-benzoyl-3-O-methyl-α-L-idopyranosyluronate)-(1→4)-(6-O-acetyl-2-O-butyl-3-O-methyl-α-D-glucopyranosyl)-(1→4)-(methyl 2-O-benzoyl-3-O-methyl-α-L-idopyranosyluronate)-(1→4)-(2-O-butyl-3-O-methyl-6-O-tert-butyldiphenylsilyl-α-D-glucopyranosyl)-(1→4)-(methyl 2-O-benzoyl-3-O-methyl-α-L-idopyranosyluronate)-(1→4)-2-O-butyl-3-O-methyl-6-O-tert-butyldiphenylsilyl-β-D-glucopyranoside (108)

Imidazole (1.3 g, 19.25 mmol) and tert-butyldiphenylsilyl chloride (2.5 ml, 9.52 mmol) are successively added, at ambient temperature and under an argon atmosphere, to a solution of compound 107 (1.09 g, 0.35 mmol) in N,N-dimethyl-formamide (4.2 ml). After stirring at 60° C. for 22 h, the progression of the reaction is stopped by adding methanol (425 μl, 10.47 mmol). The organic phase is washed with a 10% aqueous solution of potassium hydrogen sulphate, dried over sodium sulphate, filtered and then evaporated under vacuum. The residue is purified by flash chromatography on a silica gel column (cyclohexane/acetone), to give 860 mg of compound 108.

SFC-MS m/z 1582 [(M+2H+CH₃CN)^{2+]}/2. T_{R1}=8.52 min

### 5-Phenylpentyl (methyl 3-O-methyl-4-O-tert-butyldiphenylsilyl-α-L-idopyranosyluronate)-(1→4)-(2-O-butyl-3-O-methyl-α-D-glucopyranosyl)-(1→4)-(methyl 3-O-methyl-α-L-idopyranosyluronate)-(1→4)-(2-O-butyl-3-O-methyl-α-D-glucopyranosyl)-(1→4)-(methyl 3-O-methyl-α-L-idopyranosyluronate)-(1→4)-(2-O-butyl-3-O-methyl-6-O-tert-butyldiphenylsilyl-α-D-glucopyranosyl)-(1→4)-(methyl 3-O-methyl-α-L-idopyranosyluronate)-(1→4)-2-O-butyl-3-O-methyl-6-O-tert-butyldiphenylsilyl-β-D-glucopyranoside (109)

Potassium tert-butoxide (4.1 mg, 0.034 mmol) is added, at 0° C. under an argon atmosphere, to a solution of compound 108 (350 mg, 0.112 mmol) in 1/1 methanol/dioxane (728 μl). After stirring at 0° C. for 77 h, the reaction medium is neutralized by adding Dowex AG 50 WX4 H⁺ resin, filtered and then concentrated. The residue is chromatographed on a silica gel column (diisopropyl ether/acetone). The mixture obtained is again processed under the conditions described above (1/1 methanol/dioxane (460 μl), potassium tert-butoxide (2.4 mg, 0.0211 mmol), stirring at 0° C. for 48 h, then neutralization by adding Dowex AG 50 WX4 H⁺ resin, filtration and then concentration). The residue is chromatographed on a silica gel column (diisopropyl ether/acetone), to give 147 mg of compound 109.

SFC-MS m/z 1332 [(M+2H+CH₃CN)²⁺]/2. T_{R}=10.58 min.

### 5-Phenylpentyl (methyl 3-O-methyl-4-O-tert-butyldiphenylsilyl-2-O-triethylammonium sulphonato-α-L-idopyranosyluronate)-(1→4)-(2-O-butyl-3-O-methyl-6-O-triethylammonium sulphonato-α-D-glucopyranosyl)-(1→4)-(methyl 3-O-methyl-2-O-triethylammonium sulphonato-α-L-idopyranosyluronate)-(1→4)-(2-O-butyl-3-O-methyl-6-O-triethylammonium sulphonato-α-D-glucopyranosyl)-(1→4)-(methyl 3-O-methyl-2-O-triethylammonium sulphonato-α-L-idopyranosyluronate)-(1→4)-(2-O-butyl-3-O-methyl-6-O-tert-butyldiphenylsilyl-α-D-glucopyranosyl)-(1→4)-(methyl 3-O-methyl-2-O-triethylammonium sulphonato-α-L-idopyranosyluronate)-(1→4)-2-O-butyl-3-O-methyl-6-O-tert-butyldiphenylsilyl-β-D-glucopyranoside (110)

Compound 109 (147 mg, 0.056 mmol) is codistilled with anhydrous N,N-dimethylformamide

(3×5 ml) and is then placed in solution in anhydrous N,N-dimethylformamide (5 ml). The sulphur trioxide-triethylamine complex (304 mg, 1.68 mmol) is added to this solution. The mixture is stirred at 55° C. for 16 h in the dark and then the excess reagent is destroyed with methanol (273 μl, 4.98 mmol). The reaction medium is loaded onto a Sephadex® LH20 gel column (95×2 cm) eluted with a 9/1 v/v methanol/N,N-dimethylformamide mixture, to give compound 110 (172 mg).

¹H NMR [600 MHz] (CD₃OD) δ of the anomeric protons: 5.38 IdoUA^{VIII}, 5.24 Glc^{VII}, 5.34 IdoUA^{VI}, 5.29 Glc^{V}, 5.36 IdoUA^{IV}, 5.31 Glc^{III}, 5.38 IdoUA^{II}, 4.25 Glc^{I}.

### 5-Phenylpentyl (methyl 3-O-methyl-2-O-ammonium sulphonato-α-L-idopyranosyluronate)-(1→4)-(2-O-butyl-3-O-methyl-6-O-ammonium sulphonato-α-D-glucopyranosyl)-(1→4)-methyl 3-O-methyl-2-O-ammonium sulphonato-α-L-idopyranosyluronate)-(1→4)-(2-O-butyl-3-O-methyl-6-O-ammonium sulphonato-α-D-glucopyranosyl)-(1→4)-(methyl 3-O-methyl-2-O-ammonium sulphonato-α-L-idopyranosyluronate)-(1→4)-(2-O-butyl-3-O-methyl-α-D-glucopyranosyl)-(1→4)-(methyl 3-O-methyl-2-O-ammonium sulphonato-α-L-idopyranosyluronate)-(1→4)-2-O-butyl-3-O-methyl-β-D-glucopyranoside (111)

Ammonium fluoride (77 mg, 2.07 mmol) is added, under argon, to a solution of compound 110 (44.8 mg, 0.0172 mmol) in methanol (2.2 ml). After stirring at 55° C. for 24 h, the reaction medium is loaded onto a Sephadex® LH20 gel column (95×2 cm) eluted with 9/1 v/v methanol/N,N-dimethylformamide, to give compound 111 (38 mg).

¹H NMR [600 MHz] (CD₃OD) δ of the anomeric protons: 5.32 IdoUA^{VIII}, 5.29 Glc^{VII}, 5.37 IdoUA^{VI}, 5.28 Glc^{V}, 5.26 IdoUA^{IV}, 5.33 Glc^{III}, 5.28 IdoUA^{II}, 4.28 Glc^{I}.

### EXAMPLES OF COMPOUNDS ACCORDING TO THE INVENTION

### Example 1

### Methyl (sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)]₂-(sodium 3-O-methyl-2-O-sodium sulphonato-α-L-idopyranosyluronate)-(1→4)-2-acetamido-2-deoxy-3-O-methyl-6-O-sodium sulphonato-α-D-glucopyranoside (compound No. 1)

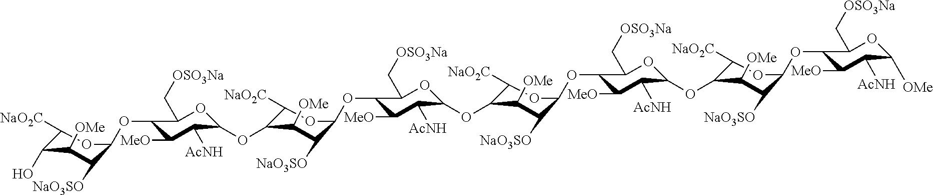

Sodium hydrogen carbonate (84 mg, 1 mmol) and then acetic anhydride (47 μl, 0.5 mmol) are added, at 0° C. under an argon atmosphere, to compound 38 (15 mg, 0.0063 mmol) dissolved in a saturated aqueous solution of sodium hydrogen carbonate (813 μl). After stirring at 0° C. for 3 h and at ambient temperature for 14 h, the reaction medium is loaded onto a Sephadex® G25-fine gel column (95×2 cm) eluted with a 0.2 M aqueous solution of NaCl. The fractions containing the expected compound are combined, and loaded onto a Sephadex® G25-fine gel column (95×2 cm) eluted with water. Compound 1 (12 mg) is obtained (12 mg) after concentration under vacuum.

¹H NMR [600 MHz] (D₂O) δ of the anomeric protons: 5.15 IdoUA^{VIII}, 5.10 Glc^{VII}, 5.17 IdoUA^{VI}, 5.11 Glc^{V}, 5.16 IdoUA^{IV}, 5.11 Glc^{III}, 5.17 IdoUA^{II}, 4.72 Glc^{I}.

ESI-MS m/z 574.06 [(M 4H)⁴⁻]

CE: T_{R}=4.70 min.

### Example 2

### Pentyl (sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)]₂-(sodium 3-O-methyl-2-O-sodium sulphonato-α-L-idopyranosyluronate)-(1→4)-2-acetamido-2-deoxy-3-O-methyl-6-O-sodium sulphonato-β-D-glucopyranoside (compound No. 2)

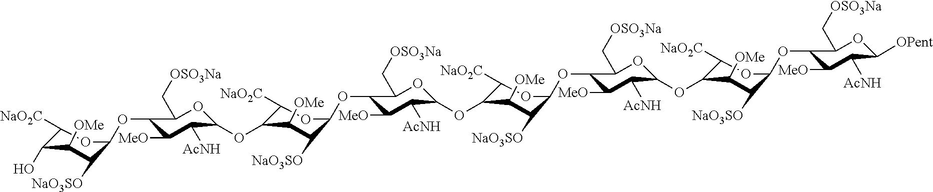

Sodium hydrogen carbonate (48 mg, 0.567 mmol) and then acetic anhydride (27 μl, 0.284 mmol) are added, at 0° C. under an argon atmosphere, to compound 53 (8.7 mg, 0.00355 mmol) dissolved in a saturated aqueous solution of sodium hydrogen carbonate (355 μl). After stirring at ambient temperature for 16 h, the reaction medium is loaded onto a Sephadex® G25-fine gel column (95×2 cm) eluted with a 0.2 M aqueous solution of NaCl. The fractions containing the expected compound are combined, and loaded onto a Sephadex® G25-fine gel column (95×2 cm) eluted with water. The residue obtained is reprocessed under the same conditions, to give 9.2 mg of compound 2.

¹H NMR [600 MHz] (D₂O) δ of the anomeric protons: 5.15 IdoUA^{VIII}, 5.09 Glc^{VII}, 5.15 IdoUA^{VI}, 5.09 Glc^{V}, 5.15 IdoUA^{IV}, 5.09 Glc^{III}, 5.15 IdoUA^{II}, 4.54 Glc^{I}.

ESI-MS m/z 588.06 [(M 4H)⁴⁻]

CE: T_{R}=4.69 min.

### Example 3

### Pentyl (sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)]₂-(sodium 3-O-methyl-2-O-sodium sulphonato-α-L-idopyranosyluronate)-(1→4)-2-butanoylamino-2-deoxy-3-O-methyl-6-O-sodium sulphonato-β-D-glucopyranoside (compound No. 3)

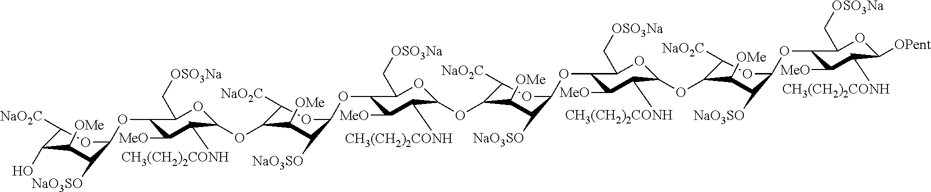

A solution of N,N-diisopropylethylamine (11 μl, 0.063 mmol) in N,N-dimethyl-formamide (33 μl) and N-butyroxysuccinimide (9 mg, 0.048 mmol), prepared according to Naito et al. Journal of Antibiotics, 29; 1976, 1286, dissolved in N,N-dimethylformamide (33 μl), are added, at 0° C. under an argon atmosphere, to compound 53 (8.9 mg, 0.00363 mmol) dissolved in N,N-dimethylformamide (471 μl) and water (290 μl). After magnetic stirring at ambient temperature for 3.5 h, two further additions of reagents are carried out (same amounts), 3 h 30 min apart. After stirring at ambient temperature for 16 h, the reaction medium is loaded onto a Sephadex® G25-fine gel column (95×2 cm) eluted with a 0.2 M aqueous solution of NaCl. The fractions containing the expected compound are combined, and loaded onto a Sephadex® G25-fine gel column (95×2 cm) eluted with water. The mixture obtained is reacted under the same conditions, to give 10.3 mg of compound 3.

¹H NMR [500 MHz] (D₂O) δ of the anomeric protons Glc^{I} β: 4.45 ppm, IdoUA^{II}: 5.06 ppm, Glc^{III} α: 4.98 ppm, IdoUA^{IV}: 5.06 ppm, Glc^{V} α: 4.98 ppm, IdoUA^{VI}: 5.06 ppm, Glc^{VII} α: 4.98 ppm and IdoUA^{VIII}: 5.05 ppm.

ESI-MS m/z 588.06 [(M 4H)⁴⁻].

### Example 4

### 5-phenylpentyl (sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl-(1→4)]₂-(sodium 3-O-methyl-2-O-sodium sulphonato-α-L-idopyranosyluronate)-(1→4)-2-acetamido-2-deoxy-3-O-methyl-6-O-sodium sulphonato-β-D-glucopyranoside (compound No. 4)

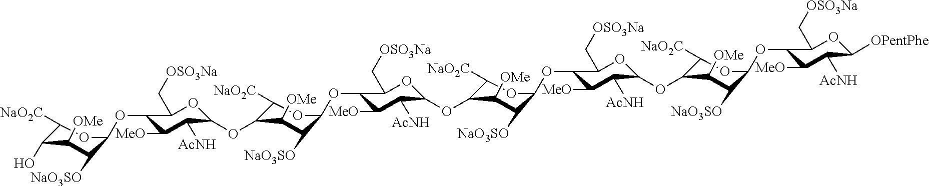

Compound 64 (6.0 mg, 0.0024 mmol) is processed according to the same procedure as that described for the preparation of Example 1, to give compound 4 (6.3 mg).

¹H NMR [500 MHz] (D₂O) δ of the anomeric protons Glc^{I} β: 4.59 ppm, IdoUA^{II}: 5.20 ppm, Glc^{III} α: 5.15 ppm, IdoUA^{IV}: 5.20 ppm, Glc^{V} α: 5.15 ppm, IdoUA^{VI}: 5.21 ppm, Glc^{VII} α: 5.15 ppm and IdoUA^{VIII}: 5.19 ppm.

ESI-MS m/z 494.25[(M 5H)⁵⁻].

### Example 5

### 5-phenylpentyl (sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)]₂-(sodium 3-O-methyl-2-O-sodium sulphonato-α-L-idopyranosyluronate)-(1→4)-2-butanoylamino-2-deoxy-3-O-methyl-6-O-sodium sulphonato-β-D-glucopyranoside (compound No. 5)

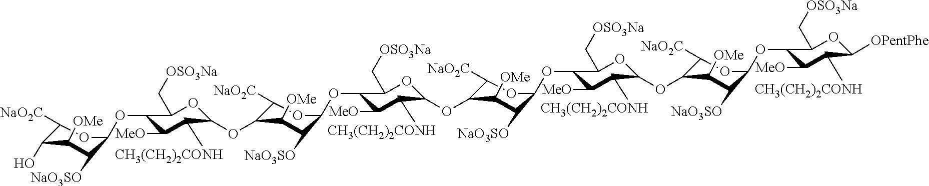

Compound 64 (25.8 mg, 0.0102 mmol) is processed according to the same procedure as that described for the preparation of Example 3, to give compound 5 (23.7 mg).

¹H NMR [500 MHz] (CDCl₃) δ of the anomeric protons Glc^{I} β: 4.51 ppm, IdoUA^{II}: 5.18 ppm, Glc^{III} α: 5.07 ppm, IdoUA^{IV}: 5.19 ppm, Glc^{V} α: 5.07 ppm, IdoUA^{VI}: 5.20 ppm, Glc^{VII} α: 5.07 ppm and IdoUA^{VIII}: 5.15 ppm.

ESI-MS m/z 507.88[(M 5H)⁵⁻].

### Example 6

### 5-phenylpentyl (sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-butanoylamino-2-deoxy-3-O-methyl-α-D-glucopyranoside)-(1→4)-(sodium 3-O-methyl-2-O-sodium sulphonato-α-L-idopyranosyluronate)-(1→4)-2-(butanoylamino)-2-deoxy-3-O-methyl-β-D-glucopyranoside (compound No. 6)

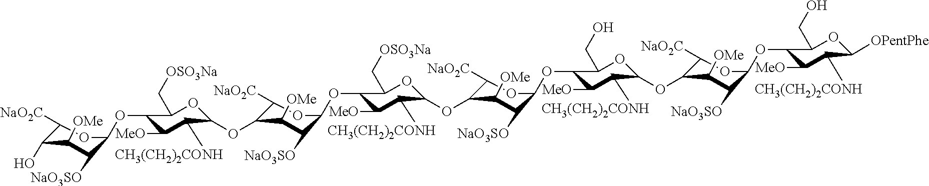

Compound 82 (55.5 mg, 0.0239 mmol) is processed according to the same procedure as that described for the preparation of Example 1, to give compound 6 (66.2 mg).

¹H NMR [600 MHz] (D₂O) δ of the anomeric protons Glc^{I} β: 4.58 IdoUA^{II}: 5.23, Glc^{III} α: 5.10, IdoUA^{IV}: 5.24, Glc^{V} α: 5.10, IdoUA^{VI}: 5.23, Glc^{VII} α: 5.13 and IdoUA^{VIII}: 5.20

CE: T_{R}=3.40 min.

### Example 7

### 5-phenylpentyl (sodium 3-O-methyl-2-O-sodium sulphonato-α-L-idopyranosyluronate)-(1→4)-(2-[(3-methylbutanoyl)amino]-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-[(3-methylbutanoyl)amino]-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-[(3-methylbutanoyl)amino]-2-deoxy-3-O-methyl-α-D-glucopyranoside)-(1→4)-(sodium 3-O-methyl-2-O-sodium sulphonato-α-L-idopyranosyluronate)-(1→4)-2-[(3-methylbutanoyl)amino]-2-deoxy-3-O-methyl-β-D-glucopyranoside (compound No. 7)

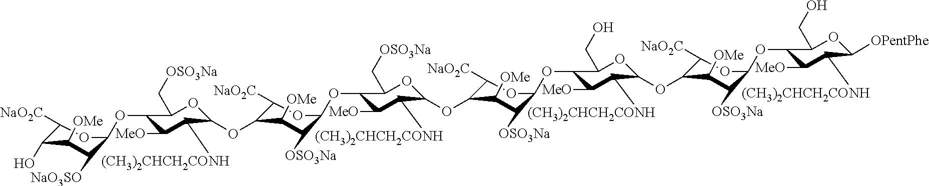

Compound 82 (49.8 mg, 0.0214 mmol) is processed according to the same procedure as that described for the preparation of Example 1, to give compound 7 (34.0 mg).

¹H NMR [600 MHz] (D₂O) δ of the anomeric protons Glc^{I} β: 4.57, IdoUA^{II}: 5.23, Glc^{III} α: 5.09, IdoUA^{IV}: 5.27, Glc^{II} α: 5.09, IdoUA^{VI}: 5.25, Glc^{VII} α: 5.12 and IdoUA^{VIII}: 5.21.

ESI-MS m/z 642.32 [(M 4H)⁴⁻].

### Example 8

### 5-phenylpentyl (sodium 3-O-methyl-2-O-sodium sulphonato-α-L-idopyranosyluronate)-(1→4)-(2-O-butyl-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-O-butyl-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-O-butyl-3-O-methyl-α-D-glucopyranosyl)-(1→4)-(sodium 3-O-methyl-2-O-sodium sulphonato-α-L-idopyranosyluronate)-(1→4)-2-O-butyl-3-O-methyl-β-D-glucopyranoside (compound No. 8)

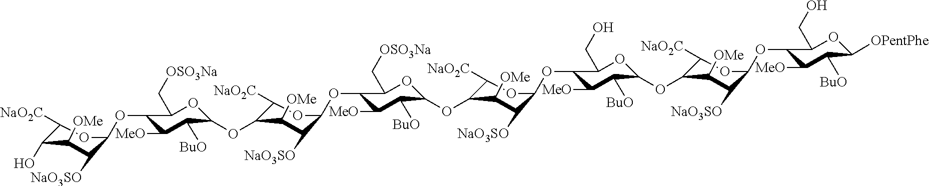

A 4.2 M aqueous solution of lithium hydroxide (290 μl, 1.216 mmol) and a 30% hydrogen peroxide solution (373 μl, 3.648 mmol) are added, at 0° C. under argon, to compound 111 (38 mg, 0.0152 mmol) dissolved in water (555 μl). After stirring at 0° C. for 2 h, at ambient temperature for 17 h and at 45° C. for 24 h, the reaction medium is loaded onto a Sephadex® G-25 fine column (90×3 cm) eluted with a 0.2 M aqueous solution of sodium chloride. The fractions containing the product are concentrated and desalified using the same column eluted with water. After concentration to dryness, compound 8 (33 mg) is obtained.

¹H NMR [600 MHz] (CD₃OD) δ of the anomeric protons: 5.17 IdoUA^{VIII}, 5.41 Glc^{VII}, 5.17 IdoUA^{VI}, 5.40 Glc^{V}, 5.18 IdoUA^{IV}, 5.40 Glc^{III}, 5.15 IdoUA^{II}, 4.50 Glc^{I}

[α]_{D} 14° (c 0.25, H₂O).

### Example 9

### 5-phenylpentyl (sodium 3-O-methyl-2-O-sodium sulphonato-α-L-idopyranosyluronate)-(1→4)-(2-O-butyl-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-O-butyl-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-O-butyl-3-O-methyl-α-D-glucopyranosyl)-(1→4)-(sodium 3-O-methyl-2-O-sodium sulphonato-α-L-idopyranosyluronate)-(1→4)-2-O-butyl-3-O-methyl-α-D-glucopyranoside (compound No. 9)

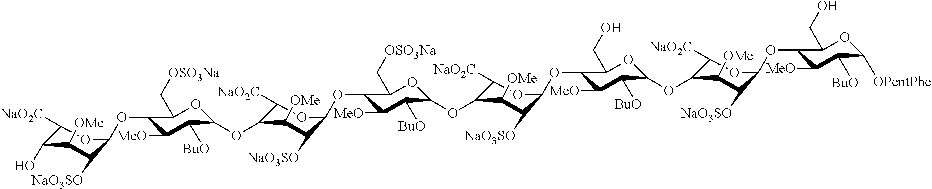

An analogous sequence was performed using compound 95α to give compound 9.

¹H NMR [600 MHz] (CD₃OD) δ of the anomeric protons: 5.17 IdoUA^{VIII}, 5.41 Glc^{VII}, 5.15 IdoUA^{VI}, 5.39 Glc^{V}, 5.17 IdoUA^{IV}, 5.40 Glc^{III}, 5.15 IdoUA^{II}, 5.09 Glc^{I}

[α]_{D} 58° (c 0.16, H₂O).

In Vitro Angiogenesis Model: Specific Activity Towards FGF2

The in vitro angiogenesis model corresponds to a rearrangement of human vein endothelial cells on a biological matrix. The matrix is prepared by distributing, into each well of a 96-well plate (Becton Dickinson 353872), 60 μl of Matrigel® diluted to 1/3 (Growth factor reduced Matrigel®: Becton Dickinson 356230) in collagen (rat Tail collagen, type I: Becton Dickinson 354249). The biological matrix hardens after 1 hour at 37° C.

Human vein endothelial cells (HUVEC ref: C-12200—Promocell) are seeded onto the biological matrix at 7800 cells/well in 120 μl of EBM® medium (Endothelial Basal Medium, Lonza C3121)+2% FCS (foetal calf serum—Lonza)+10 μg/ml hEGF (Recombinant Human Epidermal Growth Factor—Lonza). The cells are stimulated with 10 ng/ml FGF2 (R&D Systems/234—FSE-0 50) or with the products of the invention for 18 hours at 37° C. in the presence of 5% CO₂. After 24 hours, the cells are observed under a microscope (×4 objective) and the length of the pseudo-tubules is analysed using image software (Biocom Visiolab 2000 software).

In this in vitro angiogenesis test, the compounds of the invention mostly exhibit a specific activity of between 10⁻⁶ M and 10⁻¹⁰ M. For example, compounds No. 4 and 6 are active at 10⁻¹⁰ M.

Model of Cellulose Implant in Mice

This model is an adaptation of the model described by Andrade et al. (Microvascular Research, 1997, 54, 253-61) for testing pharmacological products capable of activating the onset of angiogenesis.

The animals (white inbred BALB/c J mice) are anaesthetized with a xylazine (Rompun®, 10 mg/kg)/ketamine (Imalgène® 1000, 100 mg/kg) mixture intraperitoneally. The back of the animal is shaved and disinfected with Hexomedine®. A pocket of air is created subcutaneously on the back of the mouse, by injecting 5 ml of sterile air. An incision of approximately 2 cm, on the top of the back of the animal is made in order to introduce a sterile cellulose implant (disc 1 cm in diameter, 2 mm thick, Cellspon® ref. 0501) impregnated with 50 μl of sterile solution containing the test product. The incision is then sutured and cleaned with Hexomedine®.

On the days following the insertion of the implant, the mice can receive the product into the implant via an injection through the skin (50 μl/implant/day) under gas anaesthesia (5% isoflurane (Aerrane®, Baxter)).

Seven days after the insertion of the sponge, the mice are sacrificed by means of a lethal dose of sodium pentobarbital (CEVA Santé Animale), administered intraperitoneally. The skin is then excised, approximately 1 cm around the sponge, while avoiding the scar, so as to release the skin and the sponge. The sponge is then cut into several pieces and placed in a Ribolyser® tube containing 1 ml of lysis buffer (Cell Death Detection ELISA, Roche). The tubes are shaken 4 times consecutively, for 20 seconds, at force 4, using a cell mill (FastPrep® FP 120). The tubes are then centrifuged for 10 minutes at 2000 g at 20° C. and the supernatants are frozen at −20° C. until the time of the haemoglobin assay. On the day of the assay, the tubes are again centrifuged after thawing and the haemoglobin concentration is measured with the Drabkin reagent (Sigma, volume per volume) by reading on a spectrophotometer at 405 nm against a standard range of bovine haemoglobin (Sigma).

The haemoglobin concentration in each sample is expressed in mg/ml according to the polynomial regression produced from the range. The results are expressed as a mean value (±sem) for each group. The differences between the groups are tested with an ANOVA followed by a Dunnett test on the square root of the values.

In this in vivo test, the compounds of the invention that were tested demonstrated a specific activity at 45 ng/site. For example, compounds No. 1 and 3 are active at 45 ng/site.

Thus, the compounds according to the invention increase the formation of new vessels in vitro and in vivo and post-ischaemic revascularization. The compounds according to the invention can therefore be used for the preparation of medicaments that are of use for the treatment of diseases requiring activation of FGF receptors and more generally in pathological conditions requiring activation of angiogenesis, such as cicatrisation or post-ischaemic revascularization.

According to another of its aspects, a subject of the invention is therefore medicaments which comprise a compound of formula (I) according to the invention, or a pharmaceutically acceptable salt thereof.

These medicaments find their use in therapy, in the treatment of ischaemia (cardiac ischaemia, lower limb ischaemia), the treatment of diseases associated with narrowing or obstruction of the arteries or arteritis, the treatment of angina pectoris, the treatment of thromboangiitis obliterans, the treatment of atherosclerosis, and cicatrisation. It is also possible to envisage the use of the compounds of the invention for the treatment of post-angioplasty or post-endarterectomy restenosis; for these pathological conditions, the use of stents impregnated with the compounds of the invention can be envisaged.

FGFs have been shown to be protective factors in a certain number of pathological conditions such as: chronic ulcer and refractory ulcer in diabetic or nondiabetic patients, chronic or nonchronic perforations of the eardrum, periodontitis, muscle regeneration and myoblast survival, peripheral neuropathy, post-operative nerve damage, nerve deficiencies such as Parkinson's disease, Alzheimer's disease, prion disease and neuronal degeneration in alcoholics, dementia, bioartificial pancreas graft survival in diabetic patients, retinal degeneration, stromal keratitis, pigmentary retinitis, osteoarthritis, pre-eclampsia, vascular lesions and acute respiratory distress syndrome, post-traumatic cartilage and bone repair, the repair and protection of hair follicles, and the protection and regulation of hair growth.

Thus, a subject of the invention is the compounds of formula (I) defined above, for use thereof in the treatment of the pathological conditions described above.

A subject of the invention is also the use of the compounds of formula (I) defined above, for the production of a medicament intended for the treatment of the pathological conditions described above.

According to another of its aspects, the present invention relates to pharmaceutical compositions comprising, as active ingredient, a compound according to the invention. These pharmaceutical compositions contain an effective dose of at least one compound according to the invention, or a pharmaceutically acceptable salt of said compound, and also at least one pharmaceutically acceptable excipient. Said excipients are chosen according to the pharmaceutical form and the method of administration desired, from the usual excipients that are known to those skilled in the art.

In the pharmaceutical compositions of the present invention for oral, sublingual, subcutaneous, intramuscular, intravenous, topical, local, intratracheal, intranasal, transdermal or rectal administration, the active ingredient of formula (I) above or salt thereof can be administered in a unit administration form, as a mixture with conventional pharmaceutical excipients, to animals and to human beings, for the prevention or treatment of the above disorders or diseases.

The appropriate unit administration forms include oral forms such as tablets, soft or hard gel capsules, powders, granules and oral solutions or suspensions, sublingual, buccal, intratracheal, intraocular or intranasal administration forms, forms for administration by inhalation, topical, transdermal, subcutaneous, intramuscular or intravenous administration forms, rectal administration forms, and implants. For topical application, the compounds according to the invention can be used in creams, gels, ointments or lotions.

The injectable administration forms are particularly advantageous, conventionally comprising the active compound placed in solution in water for injection, in the presence of sodium chloride. The unit dose of active compound should be suitable for the desired therapeutic effect; it may, for example, be between 0.1 and 100 mg of active ingredient.

According to another of its aspects, the present invention also relates to a method for treating the pathological conditions indicated above, which comprises the administration to a patient of an effective dose of a compound according to the invention or a pharmaceutically acceptable salt thereof.

1. An oligosaccharide compound corresponding to formula (I): 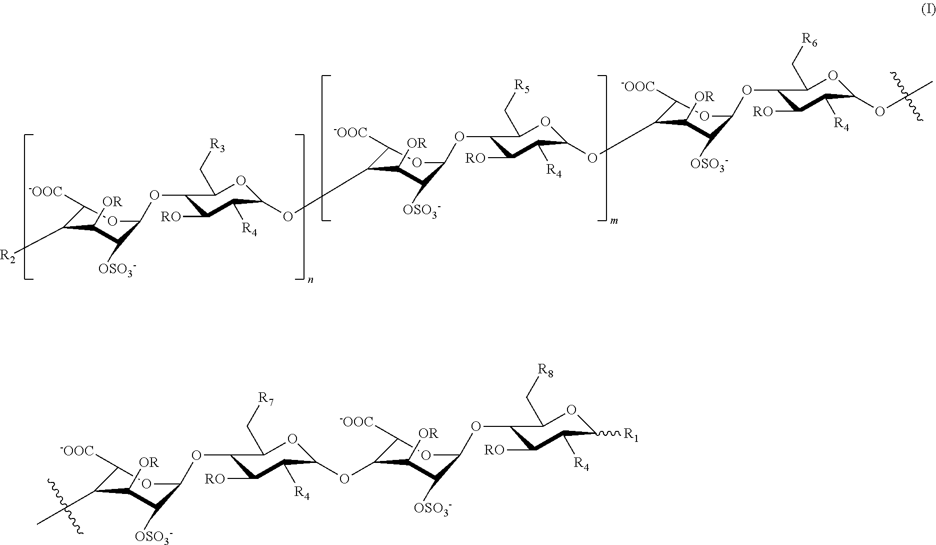
wherein:
the wavy line attached to a carbon denotes a bond located either below or above the plane of the pyranose ring of the saccharide unit,
R₁ represents an —O-alkyl group, in which said alkyl group contains from 1 to 16 carbon atoms and is optionally substituted with one or more groups, which may be identical or different, selected from aryl and cycloalkyl groups,
R₂ represents a hydroxyl group or an —O-alkyl group,
R₃, R₅, R₆, R₇ and R₈, which may be identical to or different from one another, represent an —OSO₃⁻ group or a hydroxyl group,
R₄ represents an —NH—CO-alkyl group or an —O-alkyl group,
R represents an —O-alkyl group, and
n and m, which may be identical to or different from one another, represent integers equal to 0 or 1,
in acid form or in the form of a pharmaceutically acceptable salts thereof.

2. The compound according to claim 1, in which n=1 and m=0 or n=0 and m=1.

3. The compound according to claim 1, in which R₁ represents an —O-alkyl group, where said alkyl group contains from 1 to 8 carbon atoms and is optionally substituted with 1 or 2 groups, which may be identical or different, selected from aryl groups.

4. The compound according to claim 1, in which R₁ represents an —O-methyl or —O-pentyl group and is optionally substituted with 1 or 2 phenyl groups.

5. The compound according to claim 1, in which R₃, R₅, R₆, R₇ and R₈, which may be identical to or different from one another, represent an —OSO₃⁻ group or a hydroxyl group, on the condition that at least one group among R₃, R₅, R₆, R₇ and R₈ represents an —OSO₃⁻ group.

6. The compound according to claim 1, in which R₃, R₅, R₆, R₇ and R₈ all represent —OSO₃⁻ groups.

7. The compound according to claim 1, in which at least one of the groups R₃, R₅, R₆, R₇ and R₈ represents an —OSO₃⁻ group, and at least one of the groups R₃, R₅, R₆, R₇ and R₈ represents a hydroxyl group.

8. The compound according to claim 1, in which R₃, R₅ and R₆ represent —OSO₃⁻ groups, and R₇ and R₈ represent hydroxyl groups.

9. The compound according to claim 1, in which R₄ represents an —NH—CO-alkyl group, where said alkyl group comprises from 1 to 4 carbon atoms.

10. The compound according to claim 1, in which R represents a methoxy group.

11. The compound according to claim 1, selected from the following compounds:
methyl (sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)]₂-(sodium 3-O-methyl-2-O-sodium sulphonato-α-L-idopyranosyluronate)-(1→4)-2-acetamido-2-deoxy-3-O-methyl-6-O-sodium sulphonato-α-D-glucopyranoside;
pentyl (sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)]₂-(sodium 3-O-methyl-2-O-sodium sulphonato-α-L-idopyranosyluronate)-(1→4)-2-acetamido-2-deoxy-3-O-methyl-6-O-sodium sulphonato-β-D-glucopyranoside;
pentyl (sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)]₂-(sodium 3-O-methyl-2-O-sodium sulphonato-α-L-idopyranosyluronate)-(1→4)-2-butanoylamino-2-deoxy-3-O-methyl-6-O-sodium sulphonato-β-D-glucopyranoside (No. 3);
5-phenylpentyl (sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-acetamido-2-deoxy-3-O-methyl-6-O-sodium sulphonato-α-D-glucopyranosyl)-(1→4)]₂-(sodium 3-O-methyl-2-O-sodium sulphonato-α-L-idopyranosyluronate)-(1→4)-2-acetamido-2-deoxy-3-O-methyl-6-O-sodium sulphonato-β-D-glucopyranoside;
5-phenylpentyl (sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)-[(sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)]₂-(sodium 3-O-methyl-2-O-sodium sulphonato-α-L-idopyranosyluronate)-(1→4)-2-butanoylamino-2-deoxy-3-O-methyl-6-O-sodium sulphonato-β-D-glucopyranoside;
5-phenylpentyl (sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-butanoylamino-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-butanoylamino-2-deoxy-3-O-methyl-α-D-glucopyranoside)-(1→4)-(sodium 3-O-methyl-2-O-sodium sulphonato-α-L-idopyranosyluronate)-(1→4)-2-(butanoylamino)-2-deoxy-3-O-methyl-β-D-glucopyranoside;
5-phenylpentyl (sodium 3-O-methyl-2-O-sodium sulphonato-α-L-idopyranosyluronate)-(1→4)-(2-[(3-methylbutanoyl)amino]-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-[(3-methylbutanoyl)amino]-2-deoxy-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-[(3-methylbutanoyl)amino]-2-deoxy-3-O-methyl-α-D-glucopyranoside)-(1→4)-(sodium 3-O-methyl-2-O-sodium sulphonato-α-L-idopyranosyluronate)-(1→4)-2-[(3-methylbutanoyl)amino]-2-deoxy-3-O-methyl-β-D-glucopyranoside;
5-phenylpentyl (sodium 3-O-methyl-2-O-sodium sulphonato-α-L-idopyranosyluronate)-(1→4)-(2-O-butyl-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-O-butyl-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-O-butyl-3-O-methyl-α-D-glucopyranosyl)-(1→4)-(sodium 3-O-methyl-2-O-sodium sulphonato-α-L-idopyranosyluronate)-(1→4)-2-O-butyl-3-O-methyl-β-D-glucopyranoside; and
5-phenylpentyl (sodium 3-O-methyl-2-O-sodium sulphonato-α-L-idopyranosyluronate)-(1→4)-(2-O-butyl-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-O-butyl-3-O-methyl-6-O-sodium sulphonato-α-D-glucopyranosyl)-(1→4)-(sodium 3-O-methyl-2-O-sodium sulphonato-α-L-idopyranosyluronate)-(1→4)-(2-O-butyl-3-O-methyl-α-D-glucopyranosyl)-(1→4)-(sodium 3-0-methyl-2-O-sodium sulphonato-α-L-idopyranosyluronate)-(1→4)-2-O-butyl-3-O-methyl-α-D-glucopyranoside.

12. (canceled)

13. A pharmaceutical composition comprising a compound according to claim 1, or a pharmaceutically acceptable salt thereof, and at least one pharmaceutically acceptable excipient.

14. A method for the treatment of a pathological condition requiring activation of FGF receptors, which comprises the administration to a patient of an effective dose of a compound of claim 1, or a pharmaceutically acceptable salt thereof.

15. The method of claim 14, wherein the pathological condition is selected from ischaemia, diseases associated with narrowing or obstruction of the arteries or arteritis, angina pectoris, thromboangiitis obliterans, atherosclerosis, cicatrisation, post-angioplasty or post-endarterectomy restenosis, chronic ulcer or refractory ulcer in diabetic or nondiabetic patients, chronic or nonchronic perforations of the eardrum, periodontitis, muscle regeneration, myoblast survival, peripheral neuropathy, post-operative nerve damage, nerve deficiencies, Alzheimer's disease, prion disease, neuronal degeneration in alcoholics, dementia, bioartificial pancreas graft survival in diabetic patients, retinal degeneration, stromal keratitis, pigmentary retinitis, osteoarthritis, pre-eclampsia, vascular lesions, acute respiratory distress syndrome, post-traumatic cartilage, bone repair, the repair and protection of hair follicles, and the protection and regulation of hair growth.

16. A compound of formula (II), in which Alk represents an alkyl group and Pg and Pg′ represent protecting groups:

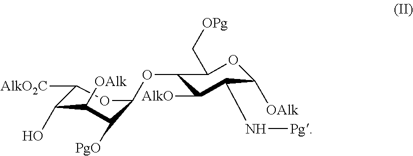

17. The compound according to claim 16, in which the Alk groups represent methyl groups, and Pg and Pg′ represent, respectively, acetyl and benzyloxycarbonyl groups.

18. A compound of formula (III), in which Alk represents an alkyl group, R₁ is as defined in claim 1, A represents an —NH-Pg″ or —O-butyl group, and Pg, Pg′ and Pg″, which may be identical to or different from one another, represent protecting groups:

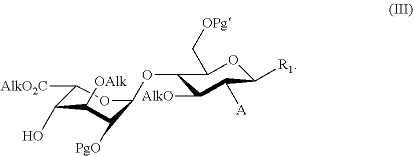

19. The compound according to claim 18, in which the Alk groups represent methyl groups, R₁ represents an —O-pentyl or —O-pentylphenyl group, Pg represents an acetyl or benzoyl group, Pg′ represents an acetyl or tert-butyldiphenylsilyl group, and A represents an —NH-benzyloxycarbonyl or —O-butyl group.

20. A compound of formula (IV), in which Alk represents an alkyl group, B represents an azide or —O-alkyl group; Pg, Pg′ and Pg″, which may be identical to or different from one another, represent protecting groups, and D represents an activating group or an —O-acetyl group:

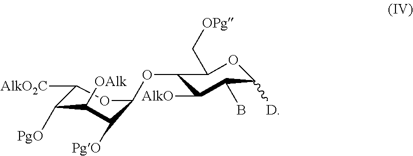

21. The compound according to claim 20, with the exception of the compound of formula (IV) in which Alk represents a methyl group, B represents an azide group, Pg represents a levulinyl group, Pg′ and Pg″ represent acetyl groups, and D represents a trichloroacetimidate group.

22. The compound according to claim 20, in which B represents an —O-alkyl group.

23. The compound according to claim 20, in which the Alk groups represent methyl groups, B represents an —O-butyl group, Pg represents a benzyl or levulinyl group, Pg′ represents an acetyl or benzoyl group, Pg″ represents an acetyl or tert-butyldiphenylsilyl group, and D represents a trichloroacetimidate or —O-acetyl group.

24. The compound according to claim 20, with the exception of the compound of formula (IV) in which Alk represents a methyl group, B represents an azide group, Pg represents a levulinyl group, Pg′ and Pg″ represent acetyl groups, and D represents a trichloroacetimidate group: wherein the Alk groups represent methyl groups, B represents an azide group, Pg represents a benzyl or levulinyl group, Pg′ represents an acetyl or benzoyl group, Pg″ represents an acetyl or tert-butyldiphenylsilyl group, and D represents a trichloroacetimidate or —O-acetyl group.
